(19) United States
(12) Patent Application Publication
(10) Pub. No.: US 2025/0144239 A1
(43) Pub. Date: May 8, 2025

(54) GENE THERAPY FOR THE TREATMENT OF WILSON'S DISEASE

(71) Applicant: LogicBio Therapeutics, Inc., Lexington, MA (US)

(72) Inventors: Shengwen Zhang, Foxborough, MA (US); Jing Liao, Lexington, MA (US); Shaobin Wang, Lexington, MA (US)

(73) Assignee: LogicBio Therapeutics, Inc., Lexington, MA (US)

(21) Appl. No.: 18/701,852

(22) PCT Filed: Oct. 18, 2022
(86) PCT No.: PCT/US2022/047007

§ 371 Date: Apr. 16, 2024

Related U.S. Application Data
(60) Provisional application No. 63/257,031, filed on Oct. 18, 2021.

(51) Int. Cl. A61K 48/00; A61K 38/46; A61P 3/00; C12N 15/86; C12N 15/90

(52) U.S. Cl. CPC A61K 48/005; A61K 38/46; A61K 48/0083; A61P 3/00; C12N 15/86; C12N 15/907; C12Y 306/03004; C12N 2750/14122; C12N 2750/14143; C12N 2750/14145

(57) ABSTRACT

The present disclosure provides compositions and methods for gene therapy. Further, the present disclosure provides compositions and methods for treatment of Wilson's Disease through novel gene therapy mechanisms. Wherein a composition of a closed circular cDNA integrating gene therapy construct, the gene therapy construct comprising, from 5′ to 3′, a polynucleotide sequence is disclosed.

### CROSS-REFERENCE TO RELATED APPLICATIONS

This application claims priority to U.S. Provisional Application No. 63/257,031, filed Oct. 18, 2021, the entirety of which is incorporated herein by reference.

### BACKGROUND

There is a subset of human diseases that can be traced to changes in the DNA that are either inherited or acquired early in embryonic development. Of particular interest for developers of genetic therapies are diseases caused by a mutation in a single gene, known as monogenic diseases. There are believed to be over 6,000 monogenic diseases. Typically, any particular genetic disease caused by inherited mutations is relatively rare, but taken together, the toll of genetic-related disease is high. Well-known genetic diseases include cystic fibrosis, Duchenne muscular dystrophy, Huntington's disease and sickle cell disease. Other classes of genetic diseases include metabolic disorders, such as organic acidemias, and lysosomal storage diseases where dysfunctional genes result in defects in metabolic processes and the accumulation of toxic byproducts that can lead to serious morbidity and mortality both in the short-term and long-term.

### SUMMARY

Monogenic diseases have been of particular interest to biomedical innovators due to the perceived simplicity of their disease pathology. However, the vast majority of these diseases and disorders remain substantially untreatable. Thus, there remains a long felt need in the art for the treatment of such diseases.

In some embodiments, the present disclosure provides methods of integrating a transgene into the genome of at least a population of cells in a tissue in a subject. In some embodiments, such methods may include a step of administering to a subject in which cells in the tissue fail to express a functional protein encoded by a gene product, a composition that delivers a transgene encoding the functional protein, wherein the composition includes: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ to the expression cassette and comprising a sequence that is substantially homologous to a genomic sequence 3′ of the target integration site in the genome of the cell, wherein, after administering the composition, the transgene is integrated into the genome of the population of cells.

In some embodiments, the present disclosure provides methods of increasing a level of expression of a transgene in a tissue over a period of time, said methods including the step of administering to a subject in need thereof a composition that delivers a transgene that integrates into the genome of at least a population of cells in the tissue of the subject, wherein the composition includes: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ to the expression cassette and comprising a sequence that is substantially homologous to a genomic sequence 3′ of the target integration site in the genome of the cell, wherein, after administering the composition, the transgene is integrated into the genome of the population of cells and the level of expression of the transgene in the tissue increases over a period of time. In some embodiments, the increased level of expression comprises an increased percent of cells in the tissue expressing the transgene.

In some embodiments, the present disclosure provides methods including a step of administering to a subject a dose of a composition that delivers to cells in a tissue of the subject a transgene, wherein the transgene (i) encodes ATP7B or a variant or truncated form thereof; (ii) integrates at a target integration site in the genome of a plurality of the cells; (iii) functionally expresses ATP7B or a variant or truncation thereof once integrated; and (iv) confers a selective advantage to the plurality of cells relative to other cells in the tissue, so that, over time, the tissue achieves a level of functional expression of ATP7B, wherein the composition comprises: a polynucleotide cassette comprising an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products when the transgene is integrated at the target integration site, a third nucleic acid sequence positioned 5′ to the expression cassette and comprising a sequence that is substantially homologous to a genomic sequence 5′ of the target integration site, and a fourth nucleic acid sequence positioned 3′ to the expression cassette and comprising a sequence that is substantially homologous to a genomic sequence 3′ of the target integration site. In some embodiments, the selective advantage comprises an increased percent of cells in the tissue expressing the transgene.

In some embodiments, the present disclosure provides methods of treatment of a monogenic disease. In some embodiments, the present disclosure provides methods of treating Wilson's Disease. In some embodiments, a method of Wilson's Disease comprises administering to a subject a dose of a composition comprising a polynucleotide cassette comprising an expression cassette comprising a first nucleic acid sequence and a second nucleic acid sequence, wherein the first nucleic acid sequence encodes a ATP7B transgene; and the second nucleic acid sequence is positioned 5′ or 3′ to the first nucleic acid sequence and promotes the production of two independent gene products when the transgene is integrated at the target integration site; a third nucleic acid sequence positioned 5′ to the expression cassette and comprising a sequence that is substantially homologous to a genomic sequence 5′ of the target integration site; and a fourth nucleic acid sequence positioned 3′ to the expression cassette and comprising a sequence that is substantially homologous to a genomic sequence 3′ of the target integration site. In some embodiments, the third nucleic acid sequence is selected from SEQ ID NO: 1, SEQ ID NO: 2, SEQ ID NO: 3, SEQ ID NO: 6, SEQ ID NO: 7, SEQ ID NO: 8, and SEQ ID NO: 12. In some embodiments, the fourth nucleic acid sequence is selected from SEQ ID NO: 4, SEQ ID NO: 5, SEQ ID NO: 9, SEQ ID NO: 10, SEQ ID NO: 11, and SEQ ID NO: 13.

In some embodiments, a composition comprises a delivery vehicle. In some embodiments, a delivery vehicle is a particle, e.g., a nanoparticle, e.g., a lipid nanoparticle. In some embodiments, a delivery vehicle is recombinant viral vector. In some embodiments, a recombinant viral vector is a recombinant AAV vector. In some embodiments, a recombinant viral vector is or comprises a capsid protein comprising an amino acid sequence having at least 95% sequence identity with the amino acid sequence of AAV8, AAV-DJ; AAV-LK03; AAV-sL65 or AAV-NP59. In some embodiments, the composition further comprises AAV2 ITR sequences. In some embodiments, the composition comprises a portion of an AAV2 ITR sequence. In some embodiments, the composition comprises an ITR having at least 80%, 85%, 90%, 95%, 99%, or 100% sequence identity to an AAV2 ITR. In some embodiments, the composition comprises ITR sequences selected from SEQ ID NO: 29, SEQ ID NO: 30, SEQ ID NO: 31, and SEQ ID NO: 32.

In accordance with various embodiments, any of a variety of transgenes may be expressed in accordance with the methods and compositions described herein. For example, in some embodiments, a transgene is or comprises an ATP7B transgene. In some embodiments, an ATP7B transgene is a wild-type human ATP7B; a codon optimized ATP7B; a synthetic ATP7B; an ATP7B variant; a truncated form of ATP7B; an ATP7B mutant, or a ATP7B fragment. In some embodiments, a transgene is or comprises a sequence with at least 80% identity to SEQ ID NO: 14 or SEQ ID NO: 15.

In some embodiments, the present invention provides recombinant viral vectors for integrating a transgene into a target integration site in the genome of a cell, including: a polynucleotide cassette comprising an expression cassette comprising a first nucleic acid sequence and a second nucleic acid sequence, wherein the first nucleic acid sequence comprises a ATP7B transgene; and the second nucleic acid sequence is positioned 5′ or 3′ to the first nucleic acid sequence and promotes the production of two independent gene products upon integration into the target integration site in the genome of the cell, a third nucleic acid sequence positioned 5′ to the expression cassette and comprising a sequence that is substantially homologous to a genomic sequence 5′ of the target integration site in the genome of the cell, and a fourth nucleic acid sequence positioned 3′ of the expression cassette and comprising a sequence that is substantially homologous to a genomic sequence 3′ of the target integration site in the genome of the cell. In some embodiments, the second nucleic acid sequence is a sequence encoding a P2A peptide. In some embodiments, the second nucleic acid sequence has at least 80% identity to SEQ ID NO: 16 or SEQ ID NO: 17. In some embodiments, the second nucleic acid sequence encodes a P2A peptide having at least 90% sequence identity to SEQ ID NO: 18. In some embodiments, provided recombinant viral vectors comprise a sequence of SEQ ID NO: 34, SEQ ID NO: 35, SEQ ID NO: 36, SEQ ID NO: 37, SEQ ID NO: 38, SEQ ID NO: 39, or SEQ ID NO: 40.

As is described herein, the present disclosure encompasses several advantageous recognitions regarding the integration of one or more transgenes into the genome of a cell. For example, in some embodiments, integration does not comprise exogenous nuclease activity.

While any application-appropriate tissue may be targeted, in some embodiments, the tissue is the liver.

As is described herein, provided methods and compositions include polynucleotide cassettes with at least four nucleic acid sequences. In some embodiments, the second nucleic acid sequence comprises: a) a nucleic acid sequence encoding a 2A peptide, b) a nucleic acid sequence encoding an internal ribosome entry site (IRES), c) a nucleic acid sequence encoding an N-terminal intein splicing region and C-terminal intein splicing region, or d) a nucleic acid sequence encoding a splice donor and a splice acceptor. In some embodiments, the third and fourth nucleic acid sequences are homology arms that integrate the transgene and the second nucleic acid sequence into a target integration site. In some embodiments a target integration site comprises an endogenous promoter and an endogenous gene. In some embodiments a target integration site is an endogenous albumin gene locus comprising an endogenous albumin promoter and an endogenous albumin gene. In some embodiments, the homology arms direct integration of the expression cassette immediately 3′ of the start codon of the endogenous albumin gene or immediately 5′ of the stop codon of the endogenous albumin gene.

In accordance with various aspects, the third and/or fourth nucleic acids may be of significant length (e.g., at least 300 nucleotides in length). In some embodiments, the third nucleic and/or fourth nucleic acid is between 100-1,400 nucleotides. In some embodiments, the third and/or fourth nucleic acid is between 300-1,000 nucleotides.

In some embodiments, a polynucleotide cassette does not comprise a promoter sequence. In some embodiments, upon integration of an expression cassette into a target integration site in the genome of the cell, the transgene is expressed under control of an endogenous promoter at the target integration site. In some embodiments, the target integration site is an albumin locus comprising an endogenous albumin promoter and an endogenous albumin gene. In some embodiments, upon integration of an expression cassette into a target integration site in the genome of a cell, the transgene is expressed under control of the endogenous albumin promoter without disruption of the endogenous albumin gene expression.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 shows measurement of ALB-2A, a biomarker for levels of ATP7B, in mice after treatment with a provided composition.

FIGS. 2A-2D show measurements of biomarkers and histology of mice treated with compositions described herein, as well as assessment of certain parameters related to such treatment. FIG. 2A shows an assessment of circulating biomarker. FIG. 2B shows an assessment of liver weight (as a % to total body weight), and liver and urinary copper (cu) levels in mice after treatment with a provided composition. FIG. 2C shows an assessment of Atp7b genomic integration levels and fused Alb-2A-Atp7b mRNA levels, circulating biomarker ALB-2A and its correlation with the amount of edited hepatocytes. FIG. 2D shows an assessment of liver morphology (hematoxylin and eosin) and human ATP7B expression (immunohistochemistry) in mice after treatment with a provided composition.

FIGS. 3A-3C show measurements of biomarkers in mice treated with compositions described herein. FIG. 3A shows an assessment of circulating biomarker. FIG. 3B shows an assessment of urinary copper (cu) levels in mice days (left) and 8 months (right) after treatment with a provided composition. FIG. 3C shows an assessment of alanine transaminase (ALT) levels in mice days (left) and 8 months (right) after treatment with a provided composition.

FIG. 4 shows measurement of ALB-2A, a biomarker for levels of ATP7B, in PXB mice after treatment with provided compositions.

FIG. 5A-5C show measurements of biomarkers and histology of mice treated with compositions described herein. Tissues were harvested at 25 weeks of age. FIG. 5A depicts exemplary images of immunohistochemical liver staining for P2A and human ATP7B. FIG. 5B depicts exemplary images of Timm's and human ATP7B immunohistochemical staining on consecutive liver slices. Numbers denote corresponding areas. FIG. 5C shows genomic DNA integration analysis (left) and fusion mRNA analysis (right). t-test: ***p<0.005; ****p<0.001.

FIG. 6A-6C show gross morphology, histology, and biomarkers of mice treated with compositions described herein. Tissues were harvested at 36 weeks of age (n=4 per group). FIG. 6A depicts exemplary photographs of livers at sacrifice. FIG. 6B depicts exemplary images of hematoxylin and eosin (H&E) and human ATP7B histochemical staining. Scale bar: 200 μm. FIG. 6C shows serum ALT levels (left) and copper content by ICP-MS in liver (middle) and urine (right). One-way ANOVA plus Tukey's post-hoc: *p<0.05; **p<0.01.

FIG. 7A-7D shows measurements of biomarkers in mice treated with compositions described herein. Tissues were harvested at 36 weeks of age (n=4 per group). FIG. 7A depicts a canonical gene therapy construct (GT) comprising a human truncated ATP7B gene (human tATP7B) expressed under control of liver specific promoter 1 (LSP1), further comprising APoE enhancer and AAT promoter sequence elements. FIG. 7B shows an assessment of urinary copper levels in mice four weeks after dosing with the gene therapy construct (WD GT) or formulation buffer (WD Vehicle), as compared to untreated, wild-type mice (Het/Wt). FIG. 7C shows an assessment of urinary copper levels in mice four weeks after dosing with the gene therapy construct (WD GT 4w) and eight weeks after dosing with the gene therapy construct (WD GT 8w), as compared to formulation buffer (WD Vehicle) and untreated, wild-type mice (Het/Wt). FIG. 7D shows liver and brain copper levels in mice two months after dosing with canonical gene therapy construct (GT) as compared to formulation buffer (Vehicle) and untreated, wild-type mice (WT).

### DEFINITIONS

In order for the present invention to be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adult: As used herein, the term “adult” refers to a human eighteen years of age or older. In some embodiments, a human adult has a weight within the range of about 90 pounds to about 250 pounds.

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to an entity whose presence, level, or form correlates with a particular biological event or state of interest, so that it is considered to be a “marker” of that event or state. Among other things, the present disclosure provides biomarkers for gene therapy (e.g., that are useful to assess one or more features or characteristics of a gene therapy treatment, such as, for instance, extent, level, and/or persistence of payload expression). In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc). In certain embodiments, the present disclosure demonstrates effectiveness of biomarkers that can be detected in a sample obtained from a subject who has received gene therapy for use in assessing one or more features or characteristics of that gene therapy; in some such embodiments, the sample is of cells, tissue, and/or fluid other than that to which the gene therapy was delivered and/or other than that where the payload is active.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Detectable Moiety: The term “detectable moiety” as used herein refers to any entity (e.g., molecule, complex, or portion or component thereof). In some embodiments, a detectable moiety is provided and/or utilizes as a discrete molecular entity; in some embodiments, it is part of and/or associated with another molecular entity. Examples of detectable moieties include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ⁹⁹mTc, ¹⁷⁷Lu, ⁸⁹Zr etc.), fluorescent dyes (for specific exemplary fluorescent dyes, see below), chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, digoxigenin, haptens, antibodies, and/or proteins for which antisera or monoclonal antibodies are available.

Child: As used herein, the term “child” refers to a human between two and 18 years of age. Body weight can vary widely across ages and specific children, with atypical range being 30 pounds to 150 pounds.

Combination therapy: As used herein, the term “combination therapy” refers to those situations in which a subject is simultaneously exposed to two or more therapeutic regimens (e.g., two or more therapeutic agents, for example a gene therapy and a non-gene therapy therapeutic modality). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time).

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Gene: As used herein, the term “gene” refers to a DNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Infant: As used herein, the term “infant” refers to a human under two years of age. Typical body weights for an infant range from 3 pounds up to 20 pounds.

Neonate: As used herein, the term “neonate” refers to a newborn human.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Peptide: As used herein, the term “peptide” or “polypeptide” refers to any polymeric chain of amino acids. In some embodiments, a peptide has an amino acid sequence that occurs in nature. In some embodiments, a peptide has an amino acid sequence that does not occur in nature. In some embodiments, a peptide has an amino acid sequence that is engineered in that it is designed and/or produced through action of the hand of man. In some embodiments, a peptide may comprise or consist of natural amino acids, non-natural amino acids, or both. In some embodiments, a peptide may comprise or consist of only natural amino acids or only non-natural amino acids. In some embodiments, a peptide may comprise D-amino acids, L-amino acids, or both. In some embodiments, a peptide may comprise only D-amino acids. In some embodiments, a peptide may comprise only L-amino acids. In some embodiments, a peptide is linear. In some embodiments, the term “peptide” may be appended to a name of a reference peptide, activity, or structure; in such instances it is used herein to refer to peptides that share the relevant activity or structure and thus can be considered to be members of the same class or family of peptides. For each such class, the present specification provides and/or those skilled in the art will be aware of exemplary peptides within the class whose amino acid sequences and/or functions are known; in some embodiments, such exemplary peptides are reference peptides for the peptide class or family. In some embodiments, a member of a peptide class or family shows significant sequence homology or identity with, shares a common sequence motif (e.g., a characteristic sequence element) with, and/or shares a common activity (in some embodiments at a comparable level or within a designated range) with a reference peptide of the class; in some embodiments with all peptides within the class). For example, in some embodiments, a member peptide shows an overall degree of sequence homology or identity with a reference peptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a variant polypeptide or nucleic acid is a truncated form of the reference polypeptide or nucleic acid. In some embodiments, a variant polypeptide that is a truncated form of the reference polypeptide may demonstrate comparable, identical, or greater levels of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. in some embodiments, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### Gene Therapy

Gene therapies alter the gene expression profile of a patient's cells by gene transfer, a process of delivering a therapeutic gene, called a transgene. Various delivery vehicles are known to be used as vectors to transport transgenes into the nucleus of a cell to alter or augment the cell's capabilities (e.g., proteome, functionality, etc.). Developers have made great strides in introducing genes into cells in tissues such as the liver, the retina of the eye and the blood-forming cells of the bone marrow using a variety of vectors. These approaches have in some cases led to approved therapies and, in other cases, have shown very promising results in clinical trials.

There are multiple gene therapy approaches. In conventional AAV gene therapy, the transgene is introduced into the nucleus of the host cell, but is not intended to integrate in chromosomal DNA. The transgene is expressed from a non-integrated genetic element called an episome that exists inside the nucleus. A second type of gene therapy employs the use of a different type of virus, such as lentivirus, that inserts itself, along with the transgene, into the chromosomal DNA but at arbitrary sites.

Episomal expression of a gene must be driven by an exogenous promoter, leading to production of a protein that corrects or ameliorates the disease condition.

### Limitations of Gene Therapy

Dilution effects as cells divide and tissues grow. In the case of gene therapy based on episomal expression, when cells divide during the process of growth or tissue regeneration, the benefits of the therapy typically decline because the transgenes were not intended to integrate into the host chromosome, thus not replicated during cell division. Each new generation of cells thus further reduces the proportion of cells expressing the transgene in the target tissue, leading to the reduction or elimination of the therapeutic benefit over time.

Inability to control site of insertion. While the use of some gene therapy using viral mediated insertion has the potential to provide long-term benefit because the gene is inserted into the host chromosome, there is no ability to control where the gene is inserted, which presents a risk of disrupting an essential gene or inserting into a location that can promote undesired effects such as tumor formation. For this reason, these integrating gene therapy approaches are primarily limited to ex vivo approaches, where the cells are treated outside the body and then re-inserted.

Use of exogenous promoters increases the risk of tumor formation. A common feature of both gene therapy approaches is that the transgene is introduced into cells together with an exogenous promoter. Promoters are required to initiate the transcription and amplification of DNA to messenger RNA, or mRNA, which will ultimately be translated into protein. Expression of high levels of therapeutic proteins from a gene therapy transgene requires strong, engineered promoters. While these promoters are essential for protein expression, previous studies conducted by others in animal models have shown that non-specific integration of gene therapy vectors can result in significant increases in the development of tumors. The strength of the promoters plays a crucial role in the increase of the development of these tumors. Thus, attempts to drive high levels of expression with strong promoters may have long-term deleterious consequences.

### A. Gene Editing

Gene editing is the deletion, alteration or augmentation of aberrant genes by introducing breaks in the DNA of cells using exogenously delivered gene editing mechanisms. Most current gene editing approaches have been limited in their efficacy due to high rates of unwanted on- and off-target modifications and low efficiency of gene correction, resulting in part from the cell trying to rapidly repair the introduced DNA break. The current focus of gene editing is on disabling a dysfunctional gene or correcting or skipping an individual deleterious mutation within a gene. Due to the number of possible mutations, neither of these approaches can address the entire population of mutations within a particular genetic disease, as would be addressed by the insertion of a full corrective gene.

Unlike the gene therapy approach, gene editing allows for the repaired genetic region to propagate to new generations of cells through normal cell division. Furthermore, the desired protein can be expressed using the cell's own regulatory machinery. The traditional approach to gene editing is nuclease-based, and it uses nuclease enzymes derived from bacteria to cut the DNA at a specific place in order to cause a deletion, make an alteration or apply a corrective sequence to the body's DNA.

Once nucleases have cut the DNA, traditional gene editing techniques modify DNA using two routes: homology-directed repair, or HDR and non-homologous end joining, or NHEJ. HDR involves highly precise incorporation of correct DNA sequences complementary to a site of DNA damage. HDR has key advantages in that it can repair DNA with high fidelity and it avoids the introduction of unwanted mutations at the site of correction. NHEJ is a less selective, more error-prone process that rapidly joins the ends of broken DNA, resulting in a high frequency of insertions or deletions at the break site.

### 1. Nuclease-Based Gene Editing

Nuclease-based gene editing uses nucleases, enzymes that were engineered or initially identified in bacteria that cut DNA. Nuclease-based gene editing is a two-step process. First, an exogenous nuclease, which is capable of cutting one or both strands in the double-stranded DNA, is directed to the desired site by a synthetic guide RNA and makes a specific cut. After the nuclease makes the desired cut or cuts, the cell's DNA repair machinery is activated and completes the editing process through either NHEJ or, less commonly, HDR.

NHEJ can occur in the absence of a DNA template for the cell to copy as it repairs a DNA cut. This is the primary or default pathway that the cell uses to repair double-stranded breaks. The NHEJ mechanism can be used to introduce small insertions or deletions, known as indels, resulting in the knocking out of the function of the gene. NHEJ creates insertions and deletions in the DNA due to its mode of repair and can also result in the introduction of off-target, unwanted mutations including chromosomal aberrations.

Nuclease-mediated HDR occurs with the co-delivery of the nuclease, a guide RNA and a DNA template that is similar to the DNA that has been cut. Consequently, the cell can use this template to construct reparative DNA, resulting in the replacement of defective genetic sequences with correct ones. We believe the HDR mechanism is the preferred repair pathway when using a nuclease-based approach to insert a corrective sequence due to its high fidelity. However, a majority of the repair to the genome after being cut with a nuclease continues to use the NHEJ mechanism. The more frequent NHEJ repair pathway has the potential to cause unwanted mutations at the cut site, thus limiting the range of diseases that any nuclease-based gene editing approaches can target at this time.

Traditional gene editing has used one of three nuclease-based approaches: Transcription activator-like effector nucleases, or TALENs; Clustered, Regularly Interspaced Short Palindromic Repeats Associated protein-9, or CRISPR/Cas9; and Zinc Finger Nucleases, or ZFN. While these approaches have already contributed to significant advances in research and product development, they have inherent limitations.

### 2. Limitations of Nuclease-Based Gene Editing

Nuclease-based gene editing approaches are limited by their use of bacterial nuclease enzymes to cut DNA and by their reliance on exogenous promoters for transgene expression. These limitations include:

Nucleases cause on- and off-target mutations. Conventional gene editing technologies can result in genotoxicity, including chromosomal alterations, based on the error-prone NHEJ process and potential off-target nuclease activity.

Delivery of gene editing components to cells is complex. Gene editing requires multiple components to be delivered into the same cell at the same time. This is technically challenging and currently requires the use of multiple vectors.

Bacterially derived nucleases are immunogenic. Because the nucleases used in conventional gene editing approaches are mostly bacterially derived, they have a higher potential for immunogenicity, which in turn limits their utility.

Because of these limitations, gene editing has been primarily restricted to ex vivo applications in cells, such as hematopoietic cells.

### GENERIDE™ Technology Platform

GeneRide™ is a novel AAV-based, nuclease-free, genome editing technology that precisely inserts a therapeutic transgene into the genome via homologous recombination. GeneRide™ provides durable transgene expression regardless of cell proliferation and tissue growth, and GeneRide™-corrected hepatocytes show selective expansion in the presence of intrinsic liver damage due to genetic defects (e.g., Wilson's Disease due to faulty ATP7B). Without wishing to be bound by any particular theory, it is contemplated that GENERIDE™ is a genome editing technology that harnesses homologous recombination, or HR, a naturally occurring DNA repair process that maintains the fidelity of the genome. In some embodiments, by using HR, GENERIDE™ allows insertion of transgenes into specific targeted genomic locations without using exogenous nucleases, which are enzymes engineered to cut DNA. GENERIDE™-directed transgene integration is designed to leverage endogenous promoters at these targeted locations to drive high levels of tissue-specific gene expression, without the detrimental issues that have been associated with the use of exogenous promoters.

GENERIDE™ technology is designed to precisely integrate corrective genes into a patient's genome to provide a stable therapeutic effect. Because GENERIDE™ is designed to have this durable therapeutic effect, it can be applied to targeting rare liver disorders in pediatric patients where it is critical to provide treatment early in a patient's life before irreversible disease pathology can occur. In some embodiments, described herein, compositions comprising GENERIDE™ constructs can be used for the treatment of Wilson's Disease.

GENERIDE™ platform technology has the potential to overcome some of the key limitations of both traditional gene therapy and conventional gene editing approaches in a way that is well-positioned to treat genetic diseases, particularly in pediatric patients. In some embodiments, GENERIDE™ uses an AAV vector to deliver a gene into the nucleus of the cell. It then uses HR to stably integrate the corrective gene into the genome of the recipient at a location where it is regulated by an endogenous promoter, leading to the potential for lifelong protein production, even as the body grows and changes over time, which is not feasible with conventional AAV gene therapy.

GENERIDE™ offers several key advantages over gene therapy and gene editing technologies that rely on exogenous promoters and nucleases. By harnessing the naturally occurring process of HR, GENERIDE™ does not face the same challenges associated with gene editing approaches that rely on engineered bacterial nuclease enzymes. The use of these enzymes has been associated with significantly increased risk of unwanted and potentially dangerous modifications in the host cell's DNA, which can lead to an increased risk of tumor formation. Furthermore, in contrast to conventional gene therapy, GENERIDE™ is intended to provide precise, site-specific, stable and durable integration of a corrective gene into the chromosome of a host cell. In preclinical animal studies with GENERIDE™ constructs, integration of the corrective gene in a specific location in the genome is observed. Thus, in some embodiments, methods and compositions of the present disclosure (e.g., those comprising GENERIDE™ constructs) provide a more durable approach than gene therapy technologies that do not integrate into the genome and lose their effect as cells divide. These benefits make GENERIDE™ well-positioned to treat genetic diseases, particularly in pediatric patients.

The modular approach disclosed herein can be applied to allow GENERIDE™ to deliver robust, tissue-specific gene expression that will be reproducible across different therapeutics delivered to the same tissue. In some embodiments, this approach allows leverage of common manufacturing processes and analytics across different GENERIDE™ product candidates and could shorten the development process of treatment programs.

Previous work on non-disruptive gene targeting is described in WO 2013/158309, and is incorporated herein by reference. Previous work on genome editing without exogenous nucleases is described in WO 2015/143177, and is incorporated herein by reference.

### B. Genome Editing Using GENERIDE™: Mechanism and Attributes

In some embodiments, genome editing with the GENERIDE™ platform differs from gene editing because it uses HR to deliver the corrective gene to one specific location in the genome. In some embodiments, GENERIDE™ inserts the corrective gene in a precise manner, leading to site-specific integration in the genome. In some embodiments, GENERIDE™ does not require the use of exogenous nucleases or promoters; instead, it leverages the cell's existing machinery to integrate and initiate transcription of therapeutic transgenes.

In some embodiments, GENERIDE™ comprises at least three components, each of which contributes to the potential benefits of the GENERIDE™ approach. In some embodiments, compositions and methods of the present disclosure comprise: homology arms, a transgene, and a nucleic acid that promotes the production of two independent gene products. In some embodiments, compositions and methods of the present disclosure comprise a first nucleic acid sequence encoding a transgene. In some embodiments, compositions and methods of the present disclosure comprise a second nucleic acid that promotes the production of two independent gene products (e.g., a 2A peptide). In some embodiments, the present disclosure provides and expression cassette comprising a first nucleic acid sequence and a second nucleic acid sequence as described herein.

In some embodiments, a second nucleic acid comprises a nucleic acid sequence encoding a 2A peptide; a nucleic acid sequence encoding an internal ribosome entry site (IRES); a nucleic acid sequence encoding an N-terminal intein splicing region and C-terminal intein splicing region; and/or a nucleic acid sequence encoding a splice donor and a splice acceptor. In some embodiments, compositions and methods of the present disclosure comprise a polynucleotide cassette comprising an expression cassette comprising said first nucleic acid and said second nucleic acid. In some embodiments, compositions and methods of the present disclosure comprise a third nucleic acid sequence comprising a sequence that is substantially homologous to a genomic sequence. In some embodiments, compositions and methods of the present disclosure comprise a fourth nucleic acid sequence comprising a sequence that is substantially homologous to a genomic sequence. In some embodiments, said third nucleic acid sequence is positioned 5′ to the expression cassette and comprises a sequence that is substantially homologous to a genomic sequence 5′ of a target integration site in a genome of a cell. In some embodiments, said fourth nucleic acid sequence is positioned 3′ to the expression cassette and comprises a sequence that is substantially homologous to a genomic sequence 3′ of a target integration site in the genome of the cell.

In some embodiments, a nucleic acid sequence encoding a 2A peptide has 80%, 85%, 90%, 95%, 99%, or 100% sequence identity to SEQ ID NO. 16 or SEQ ID NO. 17.

In some embodiments, a 2A peptide has 80%, 85%, 90%, 95%, 99%, or 100% sequence identity to SEQ ID NO. 18.

### Homology Arms Comprised of Hundreds of Nucleotides.

In some embodiments, methods and compositions of the present disclosure comprise flanking sequences, known as homology arms. In some embodiments, homology arms direct site-specific integration (also referred to herein as promoting integration) and limit off-target insertion of the construct. In some embodiments, said third and fourth nucleic acid sequences comprise homology arms. In some embodiments, each homology arm is hundreds of nucleotides long, in contrast to guide sequences used in CRISPR/Cas9, which are only dozens of base pairs long. In some embodiments, this increased length may promote improved precision and site-specific integration. In some embodiments, GENERIDE™'s homology arms direct integration of the transgene immediately behind a highly expressed gene. In some embodiments, integration of the transgene immediately behind a highly expressed gene results in high levels of expression without the need to introduce an exogenous promoter.

In some embodiments, a third or fourth nucleic acid is between 100-2000; 100-350; 200-450, 300-550; 400-650; 500-750; 600-850; 700-950; 800-1050; 900-1150; 1000-1250; 1100-1350; 1200-1450; 1300-1550; 1400-1650; 1500-1750; 1600-1850; 1700-1950; 1800-2050; nucleotides in length. In some embodiments, a third or fourth nucleic acid is about 300; 400; 500; 600; 700; 800; 900; 1000; 1100; 1200, 1300, or 1400 nucleotides in length.

In some embodiments, homology arms contain at least 70% homology to a target locus. In some embodiments, homology arms contain at least 80% homology to a target locus. In some embodiments, homology arms contain at least 90% homology to a target locus. In some embodiments, homology arms contain at least 95% homology to a target locus. In some embodiments, homology arms contain at least 99% homology to a target locus. In some embodiments, homology arms contain 100% homology to a target locus.

In some embodiments, homology arms are of the same length (also referred to as balanced homology arms or even homology arms). In some embodiments, homology arms are of different lengths (also referred to as unbalanced homology arms or uneven homology arms). In some embodiments, compositions comprising unbalanced homology arms of different lengths provide improved effects (e.g., increased rate of target site integration) as compared to a reference sequence or balanced homology arms. In some embodiments, compositions comprising homology arms of different lengths, wherein each homology arm is at least a certain length, provide improved effects (e.g., increased rate of target site integration) as compared to a reference sequence (e.g., a composition comprising homology arms of the same length).

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 1 and a 3′ homology arm sequence that has at least 70%, at least 75%, at least 80%, at least 85%, at least 90%, at least 95%, at least 99%, or 100% sequence identity with SEQ ID NO: 4. In some embodiments, a viral vector comprises a 5′ homology arm comprising SEQ ID NO: 1 and a 3′ homology arm comprising SEQ ID NO: 4. In some embodiments, a viral vector comprises a 5′ homology arm consisting of SEQ ID NO: 1 and a 3′ homology arm consisting of SEQ ID NO: 4.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 1 and a 3′ homology arm sequence that has at least 70%, at least 75%, at least 80%, at least 85%, at least 90%, at least 95%, at least 99%, or 100% sequence identity with SEQ ID NO: 5. In some embodiments, a viral vector comprises a 5′ homology arm comprising SEQ ID NO: 1 and a 3′ homology arm comprising SEQ ID NO: 5. In some embodiments, a viral vector comprises a 5′ homology arm consisting of SEQ ID NO: 1 and a 3′ homology arm consisting of SEQ ID NO: 5.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 2 and a 3′ homology arm sequence that has at least 70%, at least 75%, at least 80%, at least 85%, at least 90%, at least 95%, at least 99%, or 100% sequence identity with SEQ ID NO: 4. In some embodiments, a viral vector comprises a 5′ homology arm comprising SEQ ID NO: 2 and a 3′ homology arm comprising SEQ ID NO: 4. In some embodiments, a viral vector comprises a 5′ homology arm consisting of SEQ ID NO: 2 and a 3′ homology arm consisting of SEQ ID NO: 4.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 2 and a 3′ homology arm sequence that has at least 70%, at least 75%, at least 80%, at least 85%, at least 90%, at least 95%, at least 99%, or 100% sequence identity with SEQ ID NO: 5. In some embodiments, a viral vector comprises a 5′ homology arm comprising SEQ ID NO: 2 and a 3′ homology arm comprising SEQ ID NO: 5. In some embodiments, a viral vector comprises a 5′ homology arm consisting of SEQ ID NO: 2 and a 3′ homology arm consisting of SEQ ID NO: 5.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 3 and a 3′ homology arm sequence that has at least 70%, at least 75%, at least 80%, at least 85%, at least 90%, at least 95%, at least 99%, or 100% sequence identity with SEQ ID NO: 4. In some embodiments, a viral vector comprises a 5′ homology arm comprising SEQ ID NO: 3 and a 3′ homology arm comprising SEQ ID NO: 4. In some embodiments, a viral vector comprises a 5′ homology arm consisting of SEQ ID NO: 3 and a 3′ homology arm consisting of SEQ ID NO: 4.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 3 and a 3′ homology arm sequence that has at least 70%, at least 75%, at least 80%, at least 85%, at least 90%, at least 95%, at least 99%, or 100% sequence identity with SEQ ID NO: 5. In some embodiments, a viral vector comprises a 5′ homology arm comprising SEQ ID NO: 3 and a 3′ homology arm comprising SEQ ID NO: 5. In some embodiments, a viral vector comprises a 5′ homology arm consisting of SEQ ID NO: 3 and a 3′ homology arm consisting of SEQ ID NO: 5.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 6 and a 3′ homology arm sequence that has at least 70%, at least 75%, at least 80%, at least 85%, at least 90%, at least 95%, at least 99%, or 100% sequence identity with SEQ ID NO: 11. In some embodiments, a viral vector comprises a 5′ homology arm comprising SEQ ID NO: 6 and a 3′ homology arm comprising SEQ ID NO: 11. In some embodiments, a viral vector comprises a 5′ homology arm consisting of SEQ ID NO: 6 and a 3′ homology arm consisting of SEQ ID NO: 11.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 7 and a 3′ homology arm sequence that has at least 70%, at least 75%, at least 80%, at least 85%, at least 90%, at least 95%, at least 99%, or 100% sequence identity with SEQ ID NO: 10. In some embodiments, a viral vector comprises a 5′ homology arm comprising SEQ ID NO: 7 and a 3′ homology arm comprising SEQ ID NO: 10. In some embodiments, a viral vector comprises a 5′ homology arm consisting of SEQ ID NO: 7 and a 3′ homology arm consisting of SEQ ID NO: 10.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 8 and a 3′ homology arm sequence that has at least 70%, at least 75%, at least 80%, at least 85%, at least 90%, at least 95%, at least 99%, or 100% sequence identity with SEQ ID NO: 9. In some embodiments, a viral vector comprises a 5′ homology arm comprising SEQ ID NO: 8 and a 3′ homology arm comprising SEQ ID NO: 9. In some embodiments, a viral vector comprises a 5′ homology arm consisting of SEQ ID NO: 8 and a 3′ homology arm consisting of SEQ ID NO: 9.

In some embodiments, viral vectors provided herein may comprise a 5′ homology arm and a 3′ homology arm designed to a target an albumin locus. In some embodiments, viral vectors provided herein may comprise a 5′ homology arm sequence that has at least 70%, at least 75%, at least 80%, at least 85%, at least 90%, at least 95%, at least 99%, or 100% sequence identity with SEQ ID NO: 12 and a 3′ homology arm sequence that has at least 70%, at least 75%, at least 80%, at least 85%, at least 90%, at least 95%, at least 99%, or 100% sequence identity with SEQ ID NO: 13. In some embodiments, a viral vector comprises a 5′ homology arm comprising SEQ ID NO: 12 and a 3′ homology arm comprising SEQ ID NO: 13. In some embodiments, a viral vector comprises a 5′ homology arm consisting of SEQ ID NO: 12 and a 3′ homology arm consisting of SEQ ID NO: 13.

Exemplary homology arm sequences are provided below:

Mouse albumin 1 kb 5′ homology arm
(SEQ ID NO: 1)
GTAATGCATGGATCCCCTAGGGCGGCCGCCTGAAACTAGACAAAA
CCCGTGTGACTGGCATCGATTATTCTATTTGATCTAGCTAGTCCT
AGCAAAGTGACAACTGCTACTCCCCTCCTACACAGCCAAGATTCC
TAAGTTGGCAGTGGCATGCTTAATCCTCAAAGCCAAAGTTACTTG
GCTCCAAGATTTATAGCCTTAAACTGTGGCCTCACATTCCTTCCT
ATCTTACTTTCCTGCACTGGGGTAAATGTCTCCTTGCTCTTCTTG
CTTTCTGTCCTACTGCAGGGCTCTTGCTGAGCTGGTGAAGCACAA
GCCCAAGGCTACAGCGGAGCAACTGAAGACTGTCATGGATGACTT
TGCACAGTTCCTGGATACATGTTGCAAGGCTGCTGACAAGGACAC
CTGCTTCTCGACTGAGGTCAGAAACGTTTTTGCATTTTGACGATG
TTCAGTTTCCATTTTCTGTGCACGTGGTCAGGTGTAGCTCTCTGG
AACTCACACACTGAATAACTCCACCAATCTAGATGTTGTTCTCTA
CCGAGACTGAGGTCAGAAACGTTTTTGCATTTTGACGATGTTCAG
TTTCCATTTTCTGTGCACGTGGTCAGGTGTAGCTCTCTGGAACTC
ACACACTGAATAACTCCACCAATCTAGATGTTGTTCTCTACGTAA
CTGTAATAGAAACTGACTTACGTAGCTTTTAATTTTTATTTTCTG
CCACACTGCTGCCTATTAAATACCTATTATCACTATTTGGTTTCA
AATTTGTGACACAGAAGAGCATAGTTAGAAATACTTGCAAAGCCT
AGAATCATGAACTCATTTAAACCTTGCCCTGAAATGTTTCTTTTT
GAATTGAGTTATTTTACACATGAATGGACAGTTACCATTATATAT
CTGAATCATTTCACATTCCCTCCCATGGCCTAACAACAGTTTATC
TTCTTATTTTGGGCACAACAGATGTCAGAGAGCCTGCTTTAGGAA
TTCTAAGTAGAACTGTAATTAAGCAATGCAAGGCACGTACGTTTA
CTATGTCATTGCCTATGGCTATGAAGTGCAAATCCTAACAGTCCT
GCTAATACTTTTCTAACATCCATCATTTCTTTGTTTTCAGGGTCC
AAACCTTGTCACTAGATGCAAAGACGCCTTAGCC
Mouse albumin 0.6 kb 5' homology arm version 1
(SEQ ID NO: 2)
TGCTTCTCGACTGAGGTCAGAAACGTTTTTGCATTTTGACGATGT
TCAGTTTCCATTTTCTGTGCACGTGGTCAGGTGTAGCTCTCTGGA
ACTCACACACTGAATAACTCCACCAATCTAGATGTTGTTCTCTAC
GTAACTGTAATAGAAACTGACTTACGTAGCTTTTAATTTTTATTT
TCTGCCACACTGCTGCCTATTAAATACCTATTATCACTATTTGGT
TTCAAATTTGTGACACAGAAGAGCATAGTTAGAAATACTTGCAAA
GCCTAGAATCATGAACTCATTTAAACCTTGCCCTGAAATGTTTCT
TTTTGAATTGAGTTATTTTACACATGAATGGACAGTTACCATTAT
ATATCTGAATCATTTCACATTCCCTCCCATGGCCTAACAACAGTT
TATCTTCTTATTTTGGGCACAACAGATGTCAGAGAGCCTGCTTTA
GGAATTCTAAGTAGAACTGTAATTAAGCAATGCAAGGCACGTACG
TTTACTATGTCATTGCCTATGGCTATGAAGTGCAAATCCTAACAG
TCCTGCTAATACTTTTCTAACATCCATCATTTCTTTGTTTTCAGG
GTCCAAACCTTGTCACTAGATGCAAAGACGCCTTAGCC
Mouse albumin 0.6 kb 5' homology arm version 2
(SEQ ID NO: 3)
GACTGAGGTCAGAAACGTTTTTGCATTTTGACGATGTTCAGTTTC
CATTTTCTGTGCACGTGGTCAGGTGTAGCTCTCTGGAACTCACAC
ACTGAATAACTCCACCAATCTAGATGTTGTTCTCTACGTAACTGT
AATAGAAACTGACTTACGTAGCTTTTAATTTTTATTTTCTGCCAC
ACTGCTGCCTATTAAATACCTATTATCACTATTTGGTTTCAAATT
TGTGACACAGAAGAGCATAGTTAGAAATACTTGCAAAGCCTAGAA
TCATGAACTCATTTAAACCTTGCCCTGAAATGTTTCTTTTTGAAT
TGAGTTATTTTACACATGAATGGACAGTTACCATTATATATCTGA
ATCATTTCACATTCCCTCCCATGGCCTAACAACAGTTTATCTTCT
TATTTTGGGCACAACAGATGTCAGAGAGCCTGCTTTAGGAATTCT
AAGTAGAACTGTAATTAAGCAATGCAAGGCACGTACGTTTACTAT
GTCATTGCCTATGGCTATGAAGTGCAAATCCTAACAGTCCTGCTA
ATACTTTTCTAACATCCATCATTTCTTTGTTTTCAGGGTCCAAAC
CTTGTCACTAGATGCAAAGACGCCTTAGCC
Mouse albumin 0.6 kb 3' homology arm version 1
(SEQ ID NO: 4)
TAAACACATCACAACCACAACCTTCTCAGGTAACTATACTTGGGA
CTTAAAAAACATAATCATAATCATTTTTCCTAAAACGATCAAGAC
TGATAACCATTTGACAAGAGCCATACAGACAAGCACCAGCTGGCA
CTCTTAGGTCTTCACGTATGGTCATCAGTTTGGGTTCCATTTGTA
GATAAGAAACTGAACATATAAAGGTCTAGGTTAATGCAATTTACA
CAAAAGGAGACCAAACCAGGGAGAGAAGGAACCAAAATTAAAAAT
TCAAACCAGAGCAAAGGAGTTAGCCCTGGTTTTGCTCTGACTTAC
ATGAACCACTATGTGGAGTCCTCCATGTTAGCCTAGTCAAGCTTA
TCCTCTGGATGAAGTTGAAACCATATGAAGGAATATTTGGGGGGT
GGGTCAAAACAGTTGTGTATCAATGATTCCATGTGGTTTGACCCA
ATCATTCTGTGAATCCATTTCAACAGAAGATACAACGGGTTCTGT
TTCATAATAAGTGATCCACTTCCAAATTTCTGATGTGCCCCATGC
TAAGCTTTAACAGAATTTATCTTCTTATGACAAAGCAGCCTCCTT
TGAAAATATAGCCAACTGCACACAGCTATG
Mouse albumin 0.6 kb 3' homology arm version 2
(SEQ ID NO: 5)
TAAACACATCACAACCACAACCTTCTCAGGTAACTATACTTGGGA
CTTAAAAAACATAATCATAATCATTTTTCCTAAAACGATCAAGAC
TGATAACCATTTGACAAGAGCCATACAGACAAGCACCAGCTGGCA
CTCTTAGGTCTTCACGTATGGTCATCAGTTTGGGTTCCATTTGTA
GATAAGAAACTGAACATATAAAGGTCTAGGTTAATGCAATTTACA
CAAAAGGAGACCAAACCAGGGAGAGAAGGAACCAAAATTAAAAAT
TCAAACCAGAGCAAAGGAGTTAGCCCTGGTTTTGCTCTGACTTAC
ATGAACCACTATGTGGAGTCCTCCATGTTAGCCTAGTCAAGCTTA
TCCTCTGGATGAAGTTGAAACCATATGAAGGAATATTTGGGGGGT
GGGTCAAAACAGTTGTGTATCAATGATTCCATGTGGTTTGACCCA
ATCATTCTGTGAATCCATTTCAACAGAAGATACAACGGGTTCTGT
TTCATAATAAGTGATCCACTTCCAAATTTCTGATGTGCCCCATGC
TAAGCTTTAACAGAATTTATCTTCTTATGACAAAGCAGCCTCCTT
TGAAAATATAGCCAACTGCACACAGCTATGTTGATCA
Human albumin 0.4 kb 5' homology arm
(SEQ ID NO: 6)
GTGATGCTTATGAATATTAATAGGAATATTTGTAAGGCCTGAAAT
ATTTTGATCATGAAATCAAAACATTAATTTATTTAAACATTTACT
TGAAATGTGGTGGTTTGTGATTTAGTTGATTTTATAGGCTAGTGG
GAGAATTTACATTCAAATGTCTAAATCACTTAAAATTGCCCTTTA
TGGCCTGACAGTAACTTTTTTTTATTCATTTGGGGACAACTATGT
CCGTGAGCTTCCGTCCAGAGATTATAGTAGTAAATTGTAATTAAA
GGATATGATGCACGTGAAATCACTTTGCAATCATCAATAGCTTCA
TAAATGTTAATTTTGTATCCTAATAGTAATGCTAATATTTTCCTA
ACATCTGTCATGTCTTTGTGTTCAGGGTAAAAAACTTGTTGCTGC
AAGTCAAGCTGCCTTAGGCTTA
Human albumin 0.6 kb 5' homology arm
(SEQ ID NO: 7)
GCATGTTTGGTTAGGCTAGGGCTTAGGGATTTATATATCAAAGGA
GGCTTTGTACATGTGGGACAGGGATCTTATTTTACAAACAATTGT
CTTACAAAATGAATAAAACAGCACTTTGTTTTTATCTCCTGCTCT
ATTGTGCCATACTGTTAAATGTTTATAATGCCTGTTCTGTTTCCA
AATTTGTGATGCTTATGAATATTAATAGGAATATTTGTAAGGCCT
GAAATATTTTGATCATGAAATCAAAACATTAATTTATTTAAACAT
TTACTTGAAATGTGGTGGTTTGTGATTTAGTTGATTTTATAGGCT
AGTGGGAGAATTTACATTCAAATGTCTAAATCACTTAAAATTGCC
CTTTATGGCCTGACAGTAACTTTTTTTTATTCATTTGGGGACAAC
TATGTCCGTGAGCTTCCGTCCAGAGATTATAGTAGTAAATTGTAA
TTAAAGGATATGATGCACGTGAAATCACTTTGCAATCATCAATAG
CTTCATAAATGTTAATTTTGTATCCTAATAGTAATGCTAATATTT
TCCTAACATCTGTCATGTCTTTGTGTTCAGGGTAAAAAACTTGTT
GCTGCAAGTCAAGCTGCCTTAGGCTTA
Human albumin 0.8 kb 5' homology arm
(SEQ ID NO: 8)
TTCAAACTCAGTGCACTTGTTGAGCTTGTGAAACACAAGCCCAAG
GCAACAAAAGAGCAACTGAAAGCTGTTATGGATGATTTCGCAGCT
TTTGTAGAGAAGTGCTGCAAGGCTGACGATAAGGAGACCTGCTTT
GCCGAGGAGGTACTACAGTTCTCTTCATTTTAATATGTCCAGTAT
TCATTTTTGCATGTTTGGTTAGGCTAGGGCTTAGGGATTTATATA
TCAAAGGAGGCTTTGTACATGTGGGACAGGGATCTTATTTTACAA
ACAATTGTCTTACAAAATGAATAAAACAGCACTTTGTTTTTATCT
CCTGCTCTATTGTGCCATACTGTTAAATGTTTATAATGCCTGTTC
TGTTTCCAAATTTGTGATGCTTATGAATATTAATAGGAATATTTG
TAAGGCCTGAAATATTTTGATCATGAAATCAAAACATTAATTTAT
TTAAACATTTACTTGAAATGTGGTGGTTTGTGATTTAGTTGATTT
TATAGGCTAGTGGGAGAATTTACATTCAAATGTCTAAATCACTTA
AAATTGCCCTTTATGGCCTGACAGTAACTTTTTTTTATTCATTTG
GGGACAACTATGTCCGTGAGCTTCCGTCCAGAGATTATAGTAGTA
AATTGTAATTAAAGGATATGATGCACGTGAAATCACTTTGCAATC
ATCAATAGCTTCATAAATGTTAATTTTGTATCCTAATAGTAATGC
TAATATTTTCCTAACATCTGTCATGTCTTTGTGTTCAGGGTAAAA
AACTTGTTGCTGCAAGTCAAGCTGCCTTAGGCTTA
Human albumin 0.4 kb 3' homology arm
(SEQ ID NO: 9)
TAACATCACATTTAAAAGCATCTCAGGTAACTATATTTTGAATTT
TTTAAAAAAGTAACTATAATAGTTATTATTAAAATAGCAAAGATT
GACCATTTCCAAGAGCCATATAGACCAGCACCGACCACTATTCTA
AACTATTTATGTATGTAAATATTAGCTTTTAAAATTCTCAAAATA
GTTGCTGAGTTGGGAACCACTATTATTTCTATTTTGTAGATGAGA
AAATGAAGATAAACATCAAAGCATAGATTAAGTAATTTTCCAAAG
GGTCAAAATTCAAAATTGAAACCAAAGTTTCAGTGTTGCCCATTG
TCCTGTTCTGACTTATATGATGCGGTACACAGAGCCATCCAAGTA
AGTGATGGCTCAGCAGTGGAATACTCTGGGAATTAGGCTGAACCA
CATGAAAGAGTGCTTTATA
Human albumin 0.6 kb 3' homology arm
(SEQ ID NO: 10)
TAACATCACATTTAAAAGCATCTCAGGTAACTATATTTTGAATTT
TTTAAAAAAGTAACTATAATAGTTATTATTAAAATAGCAAAGATT
GACCATTTCCAAGAGCCATATAGACCAGCACCGACCACTATTCTA
AACTATTTATGTATGTAAATATTAGCTTTTAAAATTCTCAAAATA
GTTGCTGAGTTGGGAACCACTATTATTTCTATTTTGTAGATGAGA
AAATGAAGATAAACATCAAAGCATAGATTAAGTAATTTTCCAAAG
GGTCAAAATTCAAAATTGAAACCAAAGTTTCAGTGTTGCCCATTG
TCCTGTTCTGACTTATATGATGCGGTACACAGAGCCATCCAAGTA
AGTGATGGCTCAGCAGTGGAATACTCTGGGAATTAGGCTGAACCA
CATGAAAGAGTGCTTTATAGGGCAAAAACAGTTGAATATCAGTGA
TTTCACATGGTTCAACCTAATAGTTCAACTCATCCTTTCCATTGG
AGAATATGATGGATCTACCTTCTGTGAACTTTATAGTGAAGAATC
TGCTATTACATTTCCAATTTGTCAACATGCTGAGCTTTAATAGGA
CTTATCTTCTTATGACAACATTTATTG
Human albumin 0.8 kb 3' homology arm
(SEQ ID NO: 11)
TAACATCACATTTAAAAGCATCTCAGGTAACTATATTTTGAATTT
TTTAAAAAAGTAACTATAATAGTTATTATTAAAATAGCAAAGATT
GACCATTTCCAAGAGCCATATAGACCAGCACCGACCACTATTCTA
AACTATTTATGTATGTAAATATTAGCTTTTAAAATTCTCAAAATA
GTTGCTGAGTTGGGAACCACTATTATTTCTATTTTGTAGATGAGA
AAATGAAGATAAACATCAAAGCATAGATTAAGTAATTTTCCAAAG
GGTCAAAATTCAAAATTGAAACCAAAGTTTCAGTGTTGCCCATTG
TCCTGTTCTGACTTATATGATGCGGTACACAGAGCCATCCAAGTA
AGTGATGGCTCAGCAGTGGAATACTCTGGGAATTAGGCTGAACCA
CATGAAAGAGTGCTTTATAGGGCAAAAACAGTTGAATATCAGTGA
TTTCACATGGTTCAACCTAATAGTTCAACTCATCCTTTCCATTGG
AGAATATGATGGATCTACCTTCTGTGAACTTTATAGTGAAGAATC
TGCTATTACATTTCCAATTTGTCAACATGCTGAGCTTTAATAGGA
CTTATCTTCTTATGACAACATTTATTGGTGTGTCCCCTTGCCTAG
CCCAACAGAAGAATTCAGCAGCCGTAAGTCTAGGACAGGCTTAAA
TTGTTTTCACTGGTGTAAATTGCAGAAAGATGATCTAAGTAATTT
GGCATTTATTTTAATAGGTTTGAAAAACACATGCCATTTTACAAA
TAAGACTTATATTTGTCCTTTTGTTTTTCAGCCTACCATGAG
Cynomolgus albumin 0.6 kb 5' homology arm
(SEQ ID NO: 12)
GCATGTTTGGTTAGGCTACGGCTTAGGGATTTATATATCAAAGGA
GACTTTGTACAAGTGGGACAGGGATCTTATTTTACAAACAATTGT
CTTACAAAATGAATAAAATAACACTTTGTTTTTATCTCCTGCTCT
ATTGTGCCATACTATTAAACGTTTATAATGCCCGTTCTGTTTCCA
AATTTGTGATACTTATGAATATTAATAGGAATATTTGTAAGGCCT
AAAATATTTTGATTATGAAATCAAAACATTAATTTATTTAAACAT
TTTCATGAAAAGTGGTGGTTTGTGGTTTAGTTGATTTTATAGATT
AGTGGGAGAATTTACATTCAAATGTCTAAATCACTTAAAATTGCC
CCTTATGGCCTGACAGTATTTTTTTTTAATTCCTTTGGGAACAAC
TATGTCCGTGAGCTTCCATCCAGAGATTATAGTAGTAAATTGGAA
TTAAAGGATATGATGCACGTGAAATCACTTTGCAATCATCAATAG
CTTCATAAATGTTAATTTTGTATCCTAATAGTAATGCTAATATTT
TCCTAACATCTGTCATGTCTTTGTATTCAGGGTCCAAAATTTGTT
GCTGCAAGTCAAGCTGCCTTAGCC
Cynomolgus albumin 0.6 kb 3' homology arm
(SEQ ID NO: 13)
TAAAAACATCACAATTAAGAACATCTCAGGTAACTATATTTTGAA
TTTTTTAAAAAAGTAACTATAACAGTTATTATTAAAATAGCAAAG
ATTGACTGACGATTTCCAAGAGCCATACAGACCAGCACCAACCAC
TATTCTAAACTATTTATATATGTACATATTAGCTTTTAAAATTCT
CAAAATAGTTGCTGAGTTGGGAACCACTATTATTTCTATTTTGTA
AATGAGAAAATGAAGATAAACATCAAAGCATAGGTTAAATAATTT
TCCAAAGGGTCAAAATTCAAAATTCAAACCAAAGTTTCAGTGTTG
CCCATTGTCCTATTTTGACTTATATGATGTGGCACACAGAGCCAT
CCAAGTAAGTGATGGCTCAGCAGGAGAATACTCTAGGAATTAGAC
TGAACCATATGTAAGAGCGCTTTATAGGACAAAAACAGTTGAATA
TCAATGATTTCACATGGATCAACCTAATAGTTCAACTCATCCTTT
CCGTTGGAGAATATGATGGATCTACCTTCTGTGAACTTTATAGTG
AACAATCTGCTATTACATTTTCAATTTGTCAACATGCTGAACTTT
AATAGGACTTATTTTCTTATGACAAAA

### Measurement of Target Site Integration

As described herein, one potential issue that may arise with traditional use of nucleases to introduce nucleic acid material into cells is a significant chance of off target integration (e.g., of a transgene into a non-target site). Accordingly, it is important to verify correct integration through one or more specifically targeted assays, as described below.

In accordance with various embodiments, rate of integration may be measured at any of a variety of points in time. In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years. In some embodiments, rates of target site integration are measured through assessment of one or more biomarkers (e.g., biomarkers comprising a 2A peptide). In some embodiments, rates of target site integration are measured through assessment of one or more isolated nucleic acids (e.g., mRNA, gDNA). In some embodiments, rates of target site integration are measured through assessment of gene expression (e.g., through immunohistochemical staining).

TABLE 1A
Exemplary methods for assessment of target site integration

| Assay | Sample | Exemplary | |
|---|---|---|---|
| type | analyzed | method | Exemplary protocol |
| Genomic DNA | Liver (frozen) | qPCR | Liver biopsy subjected to genomic DNA |
| integration rate | | | extraction. qPCR method run to detect |
| (gDNA Int %) | | | percentage of allele (e.g. albumin) containing on- |
| | | | target insertion. |
| Fused mRNA | Liver (frozen) | ddPCR | Liver biopsy subjected to RNA extraction. ddPCR |
| | | | method run to quantify the copy number of |
| | | | fused mRNA (unique chimeric mRNA transcribed |
| | | | from edited allele). This assay measures the |
| | | | transcriptional activity after target insertion. |
| ALB-2A | Plasma | ELISA | Blood collected and processed for plasma. |
| | | | Proprietary ELISA used to measure 2A-tagged |
| | | | albumin (universal circulating biomarker for |
| | | | targeted integration) This assay measures total |
| | | | protein expression after target insertion. |
| Hepatocyte | Fixed liver section | IHC | Fixed liver sectioned and stained against |
| editing % | | | transgene. Transgene-positive cells counted and |
| | | | used to calculate percentage of hepatocyte |
| | | | editing. For targeted integration into a target |
| | | | integration site in the albumin locus, transgene |
| | | | expression should be hepatocyte-specific. This |
| | | | assay focuses on per-cell target integration and is |
| | | | orthogonal to gDNA Int %, which focuses on per |
| | | | allele target integration. |
| GFP expression | Fixed cells (e.g, | ICC/IHC | Fixed cells counterstained with the nuclear dye. |
| | HepG2) and/or | | GFP+ cells imaged directly or stained using anti- |
| | fixed tissue (e.g., | | HA tag antibody. This assay measures the |
| | liver) section | | percentage of cells that express the GFP |
| | | | transgene and is an indicator of viral vector |
| | | | editing efficiency. |
| ATP7B | Fixed cells (e.g, | ICC/IHC | Fixed cells counterstained with the nuclear dye. |
| expression | HepG2) and/or | | Cells stained using anti-ATP7B antibody. This |
| | fixed tissue (e.g., | | assay measures the percentage of cells that |
| | liver) section | | express the ATP7B and is an indicator of viral |
| | | | vector editing efficiency. |

### Transgene

In some embodiments, methods and compositions of the present disclosure provide one or more transgenes (e.g., ATP7B3). In some embodiments transgenes, are chosen to integrate into a genome. In some embodiments, transgenes are functional versions of a disease associated gene found in a subjects cells. In some embodiments, combined size of the transgenes and the homology arms can be optimized to increase the likelihood that these transgenes are of a suitable sequence length to be efficiently packaged in a delivery vehicle, which can increase the likelihood that the transgenes will ultimately be delivered appropriately in the patient.

In some embodiments, a nucleotide sequence encoding a transgene is codon-optimized. In some embodiments, a nucleotide sequence encoding a transgene is codon-optimized for a certain cell type (e.g., mammalian, insect, bacterial, fungal, etc.). In some embodiments, a nucleotide sequence encoding a transgene is codon-optimized for a human cell. In some embodiments, a nucleotide sequence encoding a transgene is codon-optimized for a human cell of a particular tissue type (e.g., liver, muscle, CNS, lung).

In certain embodiments, a nucleotide sequence encoding a transgene may be codon optimized to have a nucleotide homology with a reference nucleotide sequence (e.g., a wild-type gene sequence) of less than 100%. In certain embodiments, nucleotide homology between a codon-optimized nucleotide sequence encoding a transgene and a reference nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

Exemplary transgene sequences are provided below:

Mouse truncated ATP7B
(SEQ ID NO: 14)
CCTGAACAGGAGAGACAGGTCACAGCCAAAGAGGCCAGTCGGAAA
ATCTTATCTAAACTTGCTTTGCCCGGCCGGCCCTGGGAGCAATCA
ATGAAGCAGAGCTTCGCCTTCGACAATGTTGGCTACGAAGGGGGT
CTGGACAGCACCAGCTCGTCCCCATCACAGAAGTGCTTCGTACAG
ATCAAAGGCATGACCTGTGCGTCCTGTGTGTCTAACATAGAAAGG
AGTCTCCAGAGACATGCTGGTATTCTCTCAGTGTTGGTCGCCTTG
ATGTCGGGAAAGGCAGAGGTCAAGTATGATCCGGAGATCATCCAG
TCGCCCAGGATAGCTCAGCTCATCCAGGACCTGGGCTTCGAAGCG
TCAGTCATGGAGGACAACACAGTCTCTGAAGGTGACATCGAACTG
ATTATCACAGGGATGACCTGTGCTTCCTGTGTCCACAACATAGAG
TCCAAGCTCACAAGGACAAATGGCATCACTTACGCCTCTGTGGCC
CTTGCCACCAGCAAAGCCCATGTGAAGTTCGATCCTGAAATTGTT
GGTCCCCGTGACATCATCAAGATCATTGAGGAAATTGGCTTTCAT
GCTTCCCTGGCCCAGAGAAACCCCAACGCCCATCACTTGGACCAC
AAGACGGAAATAAAACAGTGGAAGAAGTCTTTCCTGTGCAGCCTG
GTGTTCGGCATCCCCGTCATGGGATTGATGGTCTACATGTTAATC
CCCAGCAGTACGCCTCAGGAGACGATGGTCCTGGACCACAACATC
ATCCCAGGACTGTCCGTTCTCAATCTCATCTTCTTCATCTTGTGT
ACCTTTGTCCAATTTCTGGGTGGGTGGTACTTCTACGTACAAGCC
TACAAATCGCTGAGACACAGGTCCGCCAACATGGACGTACTCATC
GTGCTCGCCACAACCATTGCCTATGCCTACTCCCTGGTCATCCTG
GTGGTCGCCGTAGCCGAGAAGGCAGAGAAGAGCCCCGTGACCTTC
TTTGACACGCCCCCCATGCTCTTTGTGTTCATCGCCCTGGGACGG
TGGCTGGAACACGTGGCCAAGAGCAAAACTTCAGAAGCCCTTGCA
AAACTCATGTCACTCCAAGCCACAGAAGCCACAGTCGTGACCCTG
GGTGAGGACAACTTAATCCTCAGAGAGGAGCAGGTGCCCATGGAG
CTGGTGCAGCGAGGCGACGTCATCAAGGTTGTCCCTGGGGGCAAG
TTCCCAGTGGATGGGAAAGTCCTCGAAGGCAATACCATGGCTGAT
GAGTCCCTCATCACAGGAGAGGCCATGCCTGTCACTAAGAAACCT
GGGAGCATAGTGATTGCTGGCTCTATTAATGCTCATGGCTCTGTG
CTCCTTAAAGCTACCCATGTGGGTAATGACACAACTTTGGCTCAG
ATTGTAAAGTTGGTGGAAGAGGCCCAGATGTCAAAGGCTCCCATT
CAGCAGCTGGCTGACCGGTTCAGTGGATATTTTGTCCCATTCATC
ATCATCATTTCAACCTTGACCCTGGTGGTGTGGATCGTCATTGGC
TTTGTCGATTTCGGTGTGGTTCAGAAGTACTTTCCTAGCCCTAGC
AAGCACATCTCGCAGACAGAGGTGATCATCCGCTTTGCCTTCCAG
ACGTCCATCACTGTGCTGTGCATCGCCTGCCCCTGCTCCCTGGGG
CTGGCCACACCCACAGCAGTCATGGTGGGCACTGGGGTGGCTGCC
CAGAACGGTGTCCTAATCAAAGGAGGGAAGCCTCTGGAGATGGCA
CACAAGATAAAGACCGTTATGTTTGACAAAACGGGCACCATCACC
CACGGGGTCCCCAGAGTCATGCGGTTCCTGCTGCTCGCAGACGTG
GCCACACTCCCCCTCAGGAAGGTTCTGGCCGTGGTGGGCACCGCG
GAGGCCAGCAGCGAGCACCCCTTAGGCGTGGCCGTCACTAAATAC
TGCAAAGAGGAACTTGGGACGGAGACCCTGGGATACAGCACAGAC
TTCCAGGCAGTGCCCGGCTGTGGAATTAGCTGCAAAGTTAGCAAC
GTGGAGGGCATCCTGGCTCGCAGTGATCTGACTGCTCACCCTGTT
GGAGTTGGCAACCCTCCCACAGGGGAAGGTGCAGGTCCCCAGACC
TTCTCCGTGCTGATTGGAAACCGGGAATGGATGCGGCGAAACGGT
TTAACCATCTCCAGTGACATCAGTGACGCCATGACAGATCACGAG
ATGAAAGGACAGACGGCCATCCTGGTGGCCATTGATGGTGTGCTC
TGCGGGATGATCGCCATCGCAGATGCTGTCAAACCAGAGGCTGCC
CTGGCTATCTACACCCTGAAAAGCATGGGTGTGGATGTGGCTCTG
ATCACAGGGGACAACCGGAAGACAGCCAGAGCCATTGCTACTCAG
GTTGGCATCAACAAAGTCTTTGCGGAGGTACTGCCTTCTCACAAG
GTGGCCAAGGTCCAGGAGCTTCAGAATGAAGGGAAGAAAGTCGCC
ATGGTGGGAGATGGGGTGAATGACTCCCCAGCCCTGGCCCAGGCT
GACGTGGGCATCGCCATCGGGACTGGCACAGATGTTGCCATCGAA
GCAGCAGACGTGGTCCTGATCAGAAATGACTTATTGGACGTCGTG
GCCAGCATTCATCTCTCCAAGAGGACCGTCCGGAGGATCCGGGTC
AATCTGGTGCTGGCATTGATTTATAACATGGTTGGGATACCTATT
GCTGCAGGTGTCTTCATGCCCATTGGCATCGTGCTGCAGCCGTGG
ATGGGCTCAGCAGCCATGGCTGCGTCCTCTGTCTCTGTGGTGCTC
TCGTCTCTTCAGCTCAAGTGCTATAGAAAGCCCGACCTAGAGAGA
TATGAGGCCCAGGCCCACGGCCGCATGAAGCCCCTGAGTGCCTCC
CAAGTCAGCGTGCACATTGGCATGGATGACCGGCGTCGGGATTCT
CCCAGGGCCACCGCGTGGGACCAGGTCAGCTACGTGAGCCAAGTG
TCTCTGTCCTCCCTGACGTCAGACAGATTGTCTCGGCATGGCGGG
GCAGCAGAGGACGGTGGCGACAAATGGTCCCTGCTCCTGAGTGAC
AGGGATGAAGAGCAGTGCATCTGA
Human truncated ATP7B
(SEQ ID NO: 15)
CCTGAGCAGGAGAGACAGATCACAGCCAGAGAAGGGGCCAGTCGG
AAAATCTTATCTAAGCTTTCTTTGCCTACCCGTGCCTGGGAACCA
GCAATGAAGAAGAGTTTTGCTTTTGACAATGTTGGCTATGAAGGT
GGTCTGGATGGCCTGGGCCCTTCTTCTCAGCCGCAGAAGTGCTTC
TTACAGATCAAAGGCATGACCTGTGCATCCTGTGTGTCTAACATA
GAAAGGAATCTGCAGAAAGAAGCTGGTGTTCTCTCCGTGTTGGTT
GCCTTGATGGCAGGAAAGGCAGAGATCAAGTATGACCCAGAGGTC
ATCCAGCCCCTCGAGATAGCTCAGTTCATCCAGGACCTGGGTTTT
GAGGCAGCAGTCATGGAGGACTACGCAGGCTCCGATGGCAACATT
GAGCTGACAATCACAGGGATGACCTGCGCGTCCTGTGTCCACAAC
ATAGAGTCCAAACTCACGAGGACAAATGGCATCACTTATGCCTCC
GTTGCCCTTGCCACCAGCAAAGCCCTTGTTAAGTTTGACCCGGAA
ATTATCGGTCCACGGGATATTATCAAAATTATTGAGGAAATTGGC
TTTCATGCTTCCCTGGCCCAGAGAAACCCCAACGCTCATCACTTG
GACCACAAGATGGAAATAAAGCAGTGGAAGAAGTCTTTCCTGTGC
AGCCTGGTGTTTGGCATCCCTGTCATGGCCTTAATGATCTATATG
CTGATACCCAGCAACGAGCCCCACCAGTCCATGGTCCTGGACCAC
AACATCATTCCAGGACTGTCCATTCTAAATCTCATCTTCTTTATC
TTGTGTACCTTTGTCCAGCTCCTCGGTGGGTGGTACTTCTACGTT
CAGGCCTACAAATCTCTGAGACACAGGTCAGCCAACATGGACGTG
CTCATCGTCCTGGCCACAAGCATTGCTTATGTTTATTCTCTGGTC
ATCCTGGTGGTTGCTGTGGCTGAGAAGGCGGAGAGGAGCCCTGTG
ACATTCTTCGACACGCCCCCCATGCTCTTTGTGTTCATTGCCCTG
GGCCGGTGGCTGGAACACTTGGCAAAGAGCAAAACCTCAGAAGCC
CTGGCTAAACTCATGTCTCTCCAAGCCACAGAAGCCACCGTTGTG
ACCCTTGGTGAGGACAATTTAATCATCAGGGAGGAGCAAGTCCCC
ATGGAGCTGGTGCAGCGGGGCGATATCGTCAAGGTGGTCCCTGGG
GGAAAGTTTCCAGTGGATGGGAAAGTCCTGGAAGGCAATACCATG
GCTGATGAGTCCCTCATCACAGGAGAAGCCATGCCAGTCACTAAG
AAACCCGGAAGCACTGTAATTGCGGGGTCTATAAATGCACATGGC
TCTGTGCTCATTAAAGCTACCCACGTGGGCAATGACACCACTTTG
GCTCAGATTGTGAAACTGGTGGAAGAGGCTCAGATGTCAAAGGCA
CCCATTCAGCAGCTGGCTGACCGGTTTAGTGGATATTTTGTCCCA
TTTATCATCATCATGTCAACTTTGACGTTGGTGGTATGGATTGTA
ATCGGTTTTATCGATTTTGGTGTTGTTCAGAGATACTTTCCTAAC
CCCAACAAGCACATCTCCCAGACAGAGGTGATCATCCGGTTTGCT
TTCCAGACGTCCATCACGGTGCTGTGCATTGCCTGCCCCTGCTCC
CTGGGGCTGGCCACGCCCACGGCTGTCATGGTGGGCACCGGGGTG
GCCGCGCAGAACGGCATCCTCATCAAGGGAGGCAAGCCCCTGGAG
ATGGCGCACAAGATAAAGACTGTGATGTTTGACAAGACTGGCACC
ATTACCCATGGCGTCCCCAGGGTCATGCGGGTGCTCCTGCTGGGG
GATGTGGCCACACTGCCCCTCAGGAAGGTTCTGGCTGTGGTGGGG
ACTGCGGAGGCCAGCAGTGAACACCCCTTGGGCGTGGCAGTCACC
AAATACTGTAAAGAGGAACTTGGAACAGAGACCTTGGGATACTGC
ACGGACTTCCAGGCAGTGCCAGGCTGTGGAATTGGGTGCAAAGTC
AGCAACGTGGAAGGCATCCTGGCCCACAGTGAGCGCCCTTTGAGT
GCACCGGCCAGTCACCTGAATGAGGCTGGCAGCCTTCCCGCAGAA
AAAGATGCAGTCCCCCAGACCTTCTCTGTGCTGATTGGAAACCGT
GAGTGGCTGAGGCGCAACGGTTTAACCATTTCTAGCGATGTCAGT
GACGCTATGACAGACCACGAGATGAAAGGACAGACAGCCATCCTG
GTGGCTATTGACGGTGTGCTCTGTGGGATGATCGCAATCGCAGAC
GCTGTCAAGCAGGAGGCTGCCCTGGCTGTGCACACGCTGCAGAGC
ATGGGTGTGGACGTGGTTCTGATCACGGGGGACAACCGGAAGACA
GCCAGAGCTATTGCCACCCAGGTTGGCATCAACAAAGTCTTTGCA
GAGGTGCTGCCTTCGCACAAGGTGGCCAAGGTCCAGGAGCTCCAG
AATAAAGGGAAGAAAGTCGCCATGGTGGGGGATGGGGTCAATGAC
TCCCCGGCCTTGGCCCAGGCAGACATGGGTGTGGCCATTGGCACC
GGCACGGATGTGGCCATCGAGGCAGCCGACGTCGTCCTTATCAGA
AATGATTTGCTGGATGTGGTGGCTAGCATTCACCTTTCCAAGAGG
ACTGTCCGAAGGATACGCATCAACCTGGTCCTGGCACTGATTTAT
AACCTGGTTGGGATACCCATTGCAGCAGGTGTCTTCATGCCCATC
GGCATTGTGCTGCAGCCCTGGATGGGCTCAGCGGCCATGGCAGCC
TCCTCTGTGTCTGTGGTGCTCTCATCCCTGCAGCTCAAGTGCTAT
AAGAAGCCTGACCTGGAGAGGTATGAGGCACAGGCGCATGGCCAC
ATGAAGCCCCTGACGGCATCCCAGGTCAGTGTGCACATAGGCATG
GATGACAGGTGGCGGGACTCCCCCAGGGCCACACCATGGGACCAG
GTCAGCTATGTCAGCCAGGTGTCGCTGTCCTCCCTGACGTCCGAC
AAGCCATCTCGGCACAGCGCTGCAGCAGACGATGATGGGGACAAG
TGGTCTCTGCTCCTGAATGGCAGGGATGAGGAGCAGTACATC

Nucleic Acids that Promote the Production of Two Independent Gene Products

2A peptide for polycistronic expression. In some embodiments, methods and compositions of the present disclosure comprise a nucleic acid encoding a 2A peptide. Without wishing to be bound by any particular theory a nucleic acid sequence encoding a 2A peptide can play a number of important roles. In some embodiments, a 2A peptide facilitates polycistronic expression, which is the production of two distinct proteins from the same mRNA. This, in turn, allows integration of a transgene in a non-disruptive way by coupling transcription of the transgene to a highly expressed target gene in the tissue of interest, driven by a strong endogenous promoter. In some embodiments, liver-directed therapeutic programs the albumin locus can function as the site of integration. In some embodiments, through a process known as ribosomal skipping, the 2A peptide facilitates production of the therapeutic protein at the same level as the endogenous target gene (e.g., albumin) in each modified cell. In some embodiments, a subject's endogenous target gene (e.g., albumin) is produced normally, except for the addition of a C-terminal tag that serves as a circulating biomarker to indicate successful integration and expression of the transgene. In some embodiments, modification to the endogenous target gene (e.g., albumin) has minimal effect on its function. The 2A peptide has been incorporated into other potential therapeutics such as T cell receptor chimeric antigen receptors, or CAR-Ts (Qasim et al. Sci Transl Med 2017).

Exemplary sequences encoding a 2A peptide are provided below:

P2A nucleotide sequence version 1
(SEQ ID NO: 16)
GGAAGCGGCGCCACCAATTTCAGCCTGCTGAAACAGGCCGG
CGACGTGGAAGAGAACCCTGGCCCT
P2A nucleotide sequence version 2
(SEQ ID NO: 17)
GGCAGCGGCGCCACCAACTTCAGCCTGCTGAAACAGGCCGG
CGACGTGGAAGAGAACCCTGGCCCT
P2A peptide sequence
(SEQ ID NO: 18)
GSGATNFSLLKQAGDVEENPGP

In some embodiments, targeting a particular locus allows leverage of a strong endogenous promotor that drives a high level of production to maximize the expression of a transgene. In some embodiments, linking expression of the transgene to a highly expressed endogenous protein (e.g., albumin) can allow expression of the transgene at therapeutic levels without requiring the addition of exogenous promoters or the integration of the transgene in a majority of target cells.

This is supported by animal models of MMA, hemophilia B and Crigler-Najjar syndrome. In these models, integration of the transgene into approximately 1% of cells resulted in therapeutic benefit. In some embodiments, the strength of the albumin promoter overcomes the modest levels of integration to yield potentially therapeutic levels of transgene expression.

Without wishing to be bound by any particular theory, potential advantages of the GENERIDE™ approach include the following:

Targeted Integration of Transgene into the Genome.

Conventional gene therapy approaches deliver therapeutic transgenes to target cells. A major shortcoming with most of these approaches is that once the genes are inside the cell, they do not integrate into the host cell's chromosomes and do not benefit from the natural processes that lead to replication and segregation of DNA during cell division. This is particularly problematic when conventional gene therapies are introduced early in the patient's life, because the rapid growth of tissues during the child's normal development will result in dilution and eventual loss of the therapeutic benefit associated with the transgene. Non-integrated genes expressed outside the genome on a separate strand of DNA are called episomes. This episomal expression can be effective in the initial cells that are transduced, some of which may last for a long time or for the life of a patient. However, episomal expression is typically transient in target tissues such as the liver, in which there is high turnover of cells and which tends to grow considerably in size during the course of a pediatric patient's life. With GENERIDE™ technology, the transgene is integrated into the genome, which has the potential to provide stable and durable transgene expression as the cells divide and the tissue of the patient grows, and may result in a durable therapeutic benefit.

Transgene Expression without Exogenous Promoters.

In some embodiments, with GENERIDE™ technology, the transgene is expressed at a location where it is regulated by a potent endogenous promoter. In some embodiments homology arms can be used to insert the transgene at a precise site in the genome that is expressed under the control of a potent endogenous promoter (e.g., the albumin promoter). By not using exogenous promoters to drive expression of a transgene, this technology avoids the potential for off-target integration of promoters, which has been associated with an increased risk of cancer. In some embodiments, the choice of strong endogenous promoters will allow reaching therapeutic levels of protein expression from the transgene with the modest integration rates typical of the highly accurate and reliable process of HR.

### Nuclease-Free Genome Editing.

By harnessing the naturally occurring process of HR, GENERIDE™ is designed to avoid undesired side effects associated with exogenous nucleases used in conventional gene editing technologies. The use of these engineered enzymes has been associated with genotoxicity, including chromosomal alterations, resulting from the error-prone DNA repair of double-stranded DNA cuts. Avoiding the use of nucleases also reduces the number of exogenous components needed to be delivered to the cell.

### Payloads

In some embodiments, one or more vectors or constructs described herein may comprise a polynucleotide sequence encoding one or more payloads (e.g. comprising a transgene). In accordance with various aspects, any of a variety of payloads may be used (e.g., those with a diagnostic and/or therapeutic purpose), alone or in combination. In some embodiments, a payload may be or comprise a polynucleotide sequence encoding a peptide or polypeptide. In some embodiments, a payload is a peptide that has cell-intrinsic or cell-extrinsic activity that promotes a biological process to treat a medical condition. In some embodiments, a payload may be or comprise a transgene (also referred to herein as a gene of interest (GOI)). In some embodiments, a payload may be or comprise one or more inverted terminal repeat (ITR) sequences (e.g., one or more AAV ITRs). In some embodiments, a payload may be or comprise one or more transgenes with flanking ITR sequences. In some embodiments, a payload may be or comprise one or more heterologous nucleic acid sequences encoding a reporter gene (e.g., a fluorescent or luminescent reporter). In some embodiments, a payload may be or comprise one or more biomarkers (e.g., proxy for payload expression). In some embodiments, a payload may comprise a sequence for polycistronic expression (including, e.g., a 2A peptide, or intronic sequence, internal ribosomal entry site). In some embodiments, 2A peptides are small (e.g., approximately 18-22 amino acids) peptide sequences enabling co-expression of two or more discrete protein products within a single coding sequence. In some embodiments, 2A peptides allows co-expression of two or more discrete protein products regardless of arrangement of protein coding sequences. In some embodiments, 2A peptides are or comprise a consensus motif (e.g., DVEXNPGP). In some embodiments, 2A peptides promote protein cleavage. In some embodiments, 2A peptides are or comprise viral sequences (e.g., foot-and-mouth diseases virus (F2A), equine Rhinitis A virus, porcine teschovirus-1 (P2A), or Thosea asigna virus (T2A)).

In some embodiments, a payload may be or comprise a polynucleotide sequence, which comprises an expression cassette. In some embodiments. an expression cassette comprises a first nucleic acid sequence and a second nucleic acid sequence, wherein the first nucleic acid sequence encodes a transgene and the second nucleic acid sequence is positioned 5′ or 3′ to the first nucleic acid sequence and promotes the production of two independent gene products (e.g., a sequence encoding a 2A peptide).

### Methods of Monitoring

In some embodiments, the present disclosure provides methods of and/or otherwise assessing gene therapy. In some embodiments, the present disclosure provides for detection of products (e.g., polypeptides or nucleic acids) and/or biomarkers generated or encoded by compositions described herein. In some embodiments, presence of a product or biomarker is assessed in a biological sample taken from a subject who has received an integrating gene therapy treatment as described herein. In some embodiments, a biological sample is or comprises hair, skin, feces, blood, plasma, serum, cerebrospinal fluid, urine, saliva, tears, vitreous humor, liver biopsy or mucus.

In some embodiments, a product or biomarker is expressed intracellularly. In some embodiments, a product or biomarker is secreted extracellularly. In some embodiments, a product or biomarker comprises a 2A peptide. In some embodiments, a product or biomarker comprises albumin (e.g., a modified albumin, e.g., with a C-terminal tag). Methods of detecting various products or biomarkers are known in the art. In some embodiments, a product or biomarker is detected by an immunological assay or a nucleic acid amplification assay. In some embodiments, methods of detecting products or biomarkers are described in WO/2020/214582, the entire contents of which are incorporated herein by reference. In some embodiments, detection of products or biomarkers is performed 1, 2, 3, 4, 5, 6, 7, 8 or more weeks after the subject has received the gene therapy treatment or gene-integrating composition.

### Delivery Vehicles

There are multiple gene therapy approaches understood in the art. As such there are multiple mechanisms of delivery understood in the art. In some embodiments, a transgene is provided using a delivery vehicle. In some embodiment, compositions of the present disclosure comprise a delivery vehicle. In some embodiments, a delivery vehicle is or comprises a non-viral particle. In some embodiments, a delivery vehicle is a lipid particle (e.g., a lipid nanoparticle). Various lipid nanoparticles for delivery of nucleic acids are known in the art, for example, those described in WO2015184256; WO2013149140; WO2014089486A1; WO2009127060; WO2011071860; WO2020219941 the contents of each of which is incorporated herein by reference.

In some embodiments, a delivery vehicle is or comprises an exosome. One of skill in the art will recognize various methods of exosome production and use. Examples of such methods and uses are described in Luan et al., Acta Pharmacologica Sinica volume 38, pages 754-763 (2017).

In some embodiments, a delivery vehicle is or comprises a closed circular cDNA integrating gene therapy construct. In some embodiments, a delivery vehicle is or comprises a recombinant viral vector. In some embodiments, a recombinant viral vector is an adeno associated viral (AAV) vector. In some embodiments, a recombinant AAV vector comprises a capsid of, AAV8, AAV-DJ; AAV-LK03; AAV-sL65; or AAV-NP59. In some embodiments, a recombinant viral vector is or comprises a capsid protein comprising an amino acid sequence having at least 90%, 95%, 99%, Or 100% sequence identity with the amino acid sequence of, AAV8, AAV-DJ; AAV-LK03; AAV-sL65 or AAV-NP59.

TABLE 1

| | SEQ | |
|---|---|---|
| AAV | ID | |
| Capsid | NO. | SEQUENCE |
| AAV | 19 | ATGGCTGCTGACGGTTATCTTCCAGATTGGCTCGAGGACAACCTTTCTGAA |
| LK03 | | GGCATTCGAGAGTGGTGGGCGCTGCAACCTGGAGCCCCTAAACCCAAGGCA |
| (nucleotide) | | AATCAACAACATCAGGACAACGCTCGGGGTCTTGTGCTTCCGGGTTACAAA |
| | | TACCTCGGACCCGGCAACGGACTCGACAAGGGGGAACCCGTCAACGCAGCG |
| | | GACGCGGCAGCCCTCGAGCACGACAAGGCCTACGACCAGCAGCTCAAGGCC |
| | | GGTGACAACCCCTACCTCAAGTACAACCACGCCGACGCCGAGTTCCAGGAG |
| | | CGGCTCAAAGAAGATACGTCTTTTGGGGGCAACCTCGGGCGAGCAGTCTTC |
| | | CAGGCCAAAAAGAGGCTTCTTGAACCTCTTGGTCTGGTTGAGGAAGCGGCT |
| | | AAGACGGCTCCTGGAAAGAAGAGGCCTGTAGATCAGTCTCCTCAGGAACCG |
| | | GACTCATCATCTGGTGTTGGCAAATCGGGCAAACAGCCTGCCAGAAAAAGA |
| | | CTAAATTTCGGTCAGACTGGCGACTCAGAGTCAGTCCCAGACCCTCAACCT |
| | | CTCGGAGAACCACCAGCAGCCCCCACAAGTTTGGGATCTAATACAATGGCT |
| | | TCAGGCGGTGGCGCACCAATGGCAGACAATAACGAGGGTGCCGATGGAGTG |
| | | GGTAATTCCTCAGGAAATTGGCATTGCGATTCCCAATGGCTGGGCGACAGA |
| | | GTCATCACCACCAGCACCAGAACCTGGGCCCTGCCCACTTACAACAACCAT |
| | | CTCTACAAGCAAATCTCCAGCCAATCAGGAGCTTCAAACGACAACCACTAC |
| | | TTTGGCTACAGCACCCCTTGGGGGTATTTTGACTTTAACAGATTCCACTGC |
| | | CACTTCTCACCACGTGACTGGCAGCGACTCATTAACAACAACTGGGGATTC |
| | | CGGCCCAAGAAACTCAGCTTCAAGCTCTTCAACATCCAAGTTAAAGAGGTC |
| | | ACGCAGAACGATGGCACGACGACTATTGCCAATAACCTTACCAGCACGGTT |
| | | CAAGTGTTTACGGACTCGGAGTATCAGCTCCCGTACGTGCTCGGGTCGGCG |
| | | CACCAAGGCTGTCTCCCGCCGTTTCCAGCGGACGTCTTCATGGTCCCTCAG |
| | | TATGGATACCTCACCCTGAACAACGGAAGTCAAGCGGTGGGACGCTCATCC |
| | | TTTTACTGCCTGGAGTACTTCCCTTCGCAGATGCTAAGGACTGGAAATAAC |
| | | TTCCAATTCAGCTATACCTTCGAGGATGTACCTTTTCACAGCAGCTACGCT |
| | | CACAGCCAGAGTTTGGATCGCTTGATGAATCCTCTTATTGATCAGTATCTG |
| | | TACTACCTGAACAGAACGCAAGGAACAACCTCTGGAACAACCAACCAATCA |
| | | CGGCTGCTTTTTAGCCAGGCTGGGCCTCAGTCTATGTCTTTGCAGGCCAGA |
| | | AATTGGCTACCTGGGCCCTGCTACCGGCAACAGAGACTTTCAAAGACTGCT |
| | | AACGACAACAACAACAGTAACTTTCCTTGGACAGCGGCCAGCAAATATCAT |
| | | CTCAATGGCCGCGACTCGCTGGTGAATCCAGGACCAGCTATGGCCAGTCAC |
| | | AAGGACGATGAAGAAAAATTTTTCCCTATGCACGGCAATCTAATATTTGGC |
| | | AAAGAAGGGACAACGGCAAGTAACGCAGAATTAGATAATGTAATGATTACG |
| | | GATGAAGAAGAGATTCGTACCACCAATCCTGTGGCAACAGAGCAGTATGGA |
| | | ACTGTGGCAAATAACTTGCAGAGCTCAAATACAGCTCCCACGACTAGAACT |
| | | GTCAATGATCAGGGGGCCTTACCTGGCATGGTGTGGCAAGATCGTGACGTG |
| | | TACCTTCAAGGACCTATCTGGGCAAAGATTCCTCACACGGATGGACACTTT |
| | | CATCCTTCTCCTCTGATGGGAGGCTTTGGACTGAAACATCCGCCTCCTCAA |
| | | ATCATGATCAAAAATACTCCGGTACCGGCAAATCCTCCGACGACTTTCAGC |
| | | CCGGCCAAGTTTGCTTCATTTATCACTCAGTACTCCACTGGACAGGTCAGC |
| | | GTGGAAATTGAGTGGGAGCTACAGAAAGAAAACAGCAAACGTTGGAATCCA |
| | | GAGATTCAGTACACTTCCAACTACAACAAGTCTGTTAATGTGGACTTTACT |
| | | GTAGACACTAATGGTGTTTATAGTGAACCTCGCCCCATTGGCACCCGTTAC |
| | | CTTACCCGTCCCCTGTAA |
| AAV- | 20 | MAADGYLPDWLEDNLSEGIREWWALQPGAPKPKANQQHQDNARGLVLPGYK |
| LK03 | | YLGPGNGLDKGEPVNAADAAALEHDKAYDQQLKAGDNPYLKYNHADAEFQE |
| (protein) | | RLKEDTSFGGNLGRAVFQAKKRLLEPLGLVEEAAKTAPGKKRPVDQSPQEP |
| | | DSSSGVGKSGKQPARKRLNFGQTGDSESVPDPQPLGEPPAAPTSLGSNTMA |
| | | SGGGAPMADNNEGADGVGNSSGNWHCDSQWLGDRVITTSTRTWALPTYNNH |
| | | LYKQISSQSGASNDNHYFGYSTPWGYFDFNRFHCHFSPRDWQRLINNNWGF |
| | | RPKKLSFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FQFSYTFEDVPFHSSYAHSQSLDRLMNPLIDQYLYYLNRTQGTTSGTTNQS |
| | | RLLFSQAGPQSMSLQARNWLPGPCYRQQRLSKTANDNNNSNFPWTAASKYH |
| | | LNGRDSLVNPGPAMASHKDDEEKFFPMHGNLIFGKEGTTASNAELDNVMIT |
| | | DEEEIRTTNPVATEQYGTVANNLQSSNTAPTTRTVNDQGALPGMVWQDRDV |
| | | YLQGPIWAKIPHTDGHFHPSPLMGGFGLKHPPPQIMIKNTPVPANPPTTES |
| | | PAKFASFITQYSTGQVSVEIEWELQKENSKRWNPEIQYTSNYNKSVNVDFT |
| | | VDTNGVYSEPRPIGTRYLTRPL |
| AAV8 | 21 | ATGGCTGCCGATGGTTATCTTCCAGATTGGCTCGAGGACAACCTCTCTGAG |
| (nucleotide) | | GGCATTCGCGAGTGGTGGGCGCTGAAACCTGGAGCCCCGAAGCCCAAAGCC |
| | | AACCAGCAAAAGCAGGACGACGGCCGGGGTCTAGTGCTTCCTGGCTACAAG |
| | | TACCTCGGACCCTTCAACGGACTCGACAAGGGGGAGCCCGTCAACGCGGCG |
| | | GACGCAGCGGCCCTCGAGCACGACAAGGCCTACGACCAGCAGCTGCAGGCG |
| | | GGTGACAATCCGTACCTGCGGTATAACCACGCCGACGCCGAGTTTCAGGAG |
| | | CGTCTGCAAGAAGATACGTCTTTTGGGGGCAACCTCGGGCGAGCAGTCTTC |
| | | CAGGCCAAGAAGCGGGTTCTCGAACCTCTCGGTCTGGTTGAGGAAGGCGCT |
| | | AAGACGGCTCCTGGAAAGAAGAGACCGGTAGAGCCATCACCCCAGCGTTCT |
| | | CCAGACTCCTCTACGGGCATCGGCAAGAAAGGCCAACAGCCCGCCAGAAAA |
| | | AGACTCAATTTTGGTCAGACTGGCGACTCAGAGTCAGTTCCAGACCCTCAA |
| | | CCTCTCGGAGAACCTCCAGCAGCGCCCTCTGGTGTGGGACCTAATACAATG |
| | | GCTGCAGGCGGTGGCGCACCAATGGCAGACAATAACGAAGGCGCCGACGGA |
| | | GTGGGTAGTTCCTCGGGAAATTGGCATTGCGATTCCACATGGCTGGGCGAC |
| | | AGAGTCATCACCACCAGCACCCGAACCTGGGCCCTGCCCACCTACAACAAC |
| | | CACCTCTACAAGCAAATCTCCAACGGGACATCGGGAGGAGCCACCAACGAC |
| | | AACACCTACTTCGGCTACAGCACCCCCTGGGGGTATTTTGACTTTAACAGA |
| | | TTCCACTGCCACTTTTCACCACGTGACTGGCAGCGACTCATCAACAACAAC |
| | | TGGGGATTCCGGCCCAAGAGACTCAGCTTCAAGCTCTTCAACATCCAGGTC |
| | | AAGGAGGTCACGCAGAATGAAGGCACCAAGACCATCGCCAATAACCTCACC |
| | | AGCACCATCCAGGTGTTTACGGACTCGGAGTACCAGCTGCCGTACGTTCTC |
| | | GGCTCTGCCCACCAGGGCTGCCTGCCTCCGTTCCCGGCGGACGTGTTCATG |
| | | ATTCCCCAGTACGGCTACCTAACACTCAACAACGGTAGTCAGGCCGTGGGA |
| | | CGCTCCTCCTTCTACTGCCTGGAATACTTTCCTTCGCAGATGCTGAGAACC |
| | | GGCAACAACTTCCAGTTTACTTACACCTTCGAGGACGTGCCTTTCCACAGC |
| | | AGCTACGCCCACAGCCAGAGCTTGGACCGGCTGATGAATCCTCTGATTGAC |
| | | CAGTACCTGTACTACTTGTCTCGGACTCAAACAACAGGAGGCACGGCAAAT |
| | | ACGCAGACTCTGGGCTTCAGCCAAGGTGGGCCTAATACAATGGCCAATCAG |
| | | GCAAAGAACTGGCTGCCAGGACCCTGTTACCGCCAACAACGCGTCTCAACG |
| | | ACAACCGGGCAAAACAACAATAGCAACTTTGCCTGGACTGCTGGGACCAAA |
| | | TACCATCTGAATGGAAGAAATTCATTGGCTAATCCTGGCATCGCTATGGCA |
| | | ACACACAAAGACGACGAGGAGCGTTTTTTTCCCAGTAACGGGATCCTGATT |
| | | TTTGGCAAACAAAATGCTGCCAGAGACAATGCGGATTACAGCGATGTCATG |
| | | CTCACCAGCGAGGAAGAAATCAAAACCACTAACCCTGTGGCTACAGAGGAA |
| | | TACGGTATCGTGGCAGATAACTTGCAGCAGCAAAACACGGCTCCTCAAATT |
| | | GGAACTGTCAACAGCCAGGGGGCCTTACCCGGTATGGTCTGGCAGAACCGG |
| | | GACGTGTACCTGCAGGGTCCCATCTGGGCCAAGATTCCTCACACGGACGGC |
| | | AACTTCCACCCGTCTCCGCTGATGGGCGGCTTTGGCCTGAAACATCCTCCG |
| | | CCTCAGATCCTGATCAAGAACACGCCTGTACCTGCGGATCCTCCGACCACC |
| | | TTCAACCAGTCAAAGCTGAACTCTTTCATCACGCAATACAGCACCGGACAG |
| | | GTCAGCGTGGAAATTGAATGGGAGCTGCAGAAGGAAAACAGCAAGCGCTGG |
| | | AACCCCGAGATCCAGTACACCTCCAACTACTACAAATCTACAAGTGTGGAC |
| | | TTTGCTGTTAATACAGAAGGCGTGTACTCTGAACCCCGCCCCATTGGCACC |
| | | CGTTACCTCACCCGTAATCTGTAA |
| AAV8 | 22 | MAADGYLPDWLEDNLSEGIREWWALKPGAPKPKANQQKQDDGRGLVLPGYK |
| (protein) | | YLGPFNGLDKGEPVNAADAAALEHDKAYDQQLQAGDNPYLRYNHADAEFQE |
| | | RLQEDTSFGGNLGRAVFQAKKRVLEPLGLVEEGAKTAPGKKRPVEPSPQRS |
| | | PDSSTGIGKKGQQPARKRLNFGQTGDSESVPDPQPLGEPPAAPSGVGPNTM |
| | | AAGGGAPMADNNEGADGVGSSSGNWHCDSTWLGDRVITTSTRTWALPTYNN |
| | | HLYKQISNGTSGGATNDNTYFGYSTPWGYFDFNRFHCHFSPRDWQRLINNN |
| | | WGFRPKRLSFKLFNIQVKEVTQNEGTKTIANNLTSTIQVFTDSEYQLPYVL |
| | | GSAHQGCLPPFPADVFMIPQYGYLTLNNGSQAVGRSSFYCLEYFPSQMLRT |
| | | GNNFQFTYTFEDVPFHSSYAHSQSLDRLMNPLIDQYLYYLSRTQTTGGTAN |
| | | TQTLGFSQGGPNTMANQAKNWLPGPCYRQQRVSTTTGQNNNSNFAWTAGTK |
| | | YHLNGRNSLANPGIAMATHKDDEERFFPSNGILIFGKQNAARDNADYSDVM |
| | | LTSEEEIKTTNPVATEEYGIVADNLQQQNTAPQIGTVNSQGALPGMVWQNR |
| | | DVYLQGPIWAKIPHTDGNFHPSPLMGGFGLKHPPPQILIKNTPVPADPPTT |
| | | FNQSKLNSFITQYSTGQVSVEIEWELQKENSKRWNPEIQYTSNYYKSTSVD |
| | | FAVNTEGVYSEPRPIGTRYLTRNL |
| AAV-DJ | 23 | ATGGCTGCCGATGGTTATCTTCCAGATTGGCTCGAGGACACTCTCTCTGAA |
| (nucleotide) | | GGAATAAGACAGTGGTGGAAGCTCAAACCTGGCCCACCACCACCAAAGCCC |
| | | GCAGAGCGGCATAAGGACGACAGCAGGGGTCTTGTGCTTCCTGGGTACAAG |
| | | TACCTCGGACCCTTCAACGGACTCGACAAGGGAGAGCCGGTCAACGAGGCA |
| | | GACGCCGCGGCCCTCGAGCACGACAAAGCCTACGACCGGCAGCTCGACAGC |
| | | GGAGACAACCCGTACCTCAAGTACAACCACGCCGACGCCGAGTTCCAGGAG |
| | | CGGCTCAAAGAAGATACGTCTTTTGGGGGCAACCTCGGGCGAGCAGTCTTC |
| | | CAGGCCAAAAAGAGGCTTCTTGAACCTCTTGGTCTGGTTGAGGAAGCGGCT |
| | | AAGACGGCTCCTGGAAAGAAGAGGCCTGTAGAGCACTCTCCTGTGGAGCCA |
| | | GACTCCTCCTCGGGAACCGGAAAGGCGGGCCAGCAGCCTGCAAGAAAAAGA |
| | | TTGAATTTTGGTCAGACTGGAGACGCAGACTCAGTCCCAGACCCTCAACCA |
| | | ATCGGAGAACCTCCCGCAGCCCCCTCAGGTGTGGGATCTCTTACAATGGCT |
| | | GCAGGCGGTGGCGCACCAATGGCAGACAATAACGAGGGCGCCGACGGAGTG |
| | | GGTAATTCCTCGGGAAATTGGCATTGCGATTCCACATGGATGGGCGACAGA |
| | | GTCATCACCACCAGCACCCGAACCTGGGCCCTGCCCACCTACAACAACCAC |
| | | CTCTACAAGCAAATCTCCAACAGCACATCTGGAGGATCTTCAAATGACAAC |
| | | GCCTACTTCGGCTACAGCACCCCCTGGGGGTATTTTGACTTTAACAGATTC |
| | | CACTGCCACTTTTCACCACGTGACTGGCAGCGACTCATCAACAACAACTGG |
| | | GGATTCCGGCCCAAGAGACTCAGCTTCAAGCTCTTCAACATCCAGGTCAAG |
| | | GAGGTCACGCAGAATGAAGGCACCAAGACCATCGCCAATAACCTCACCAGC |
| | | ACCATCCAGGTGTTTACGGACTCGGAGTACCAGCTGCCGTACGTTCTCGGC |
| | | TCTGCCCACCAGGGCTGCCTGCCTCCGTTCCCGGCGGACGTGTTCATGATT |
| | | CCCCAGTACGGCTACCTAACACTCAACAACGGTAGTCAGGCCGTGGGACGC |
| | | TCCTCCTTCTACTGCCTGGAATACTTTCCTTCGCAGATGCTGAGAACCGGC |
| | | AACAACTTCCAGTTTACTTACACCTTCGAGGACGTGCCTTTCCACAGCAGC |
| | | TACGCCCACAGCCAGAGCTTGGACCGGCTGATGAATCCTCTGATTGACCAG |
| | | TACCTGTACTACTTGTCTCGGACTCAAACAACAGGAGGCACGACAAATACG |
| | | CAGACTCTGGGCTTCAGCCAAGGTGGGCCTAATACAATGGCCAATCAGGCA |
| | | AAGAACTGGCTGCCAGGACCCTGTTACCGCCAGCAGCGAGTATCAAAGACA |
| | | TCTGCGGATAACAACAACAGTGAATACTCGTGGACTGGAGCTACCAAGTAC |
| | | CACCTCAATGGCAGAGACTCTCTGGTGAATCCGGGCCCGGCCATGGCAAGC |
| | | CACAAGGACGATGAAGAAAAGTTTTTTCCTCAGAGCGGGGTTCTCATCTTT |
| | | GGGAAGCAAGGCTCAGAGAAAACAAATGTGGACATTGAAAAGGTCATGATT |
| | | ACAGACGAAGAGGAAATCAGGACAACCAATCCCGTGGCTACGGAGCAGTAT |
| | | GGTTCTGTATCTACCAACCTCCAGAGAGGCAACAGACAAGCAGCTACCGCA |
| | | GATGTCAACACACAAGGCGTTCTTCCAGGCATGGTCTGGCAGGACAGAGAT |
| | | GTGTACCTTCAGGGGCCCATCTGGGCAAAGATTCCACACACGGACGGACAT |
| | | TTTCACCCCTCTCCCCTCATGGGTGGATTCGGACTTAAACACCCTCCGCCT |
| | | CAGATCCTGATCAAGAACACGCCTGTACCTGCGGATCCTCCGACCACCTTC |
| | | AACCAGTCAAAGCTGAACTCTTTCATCACCCAGTATTCTACTGGCCAAGTC |
| | | AGCGTGGAGATCGAGTGGGAGCTGCAGAAGGAAAACAGCAAGCGCTGGAAC |
| | | CCCGAGATCCAGTACACCTCCAACTACTACAAATCTACAAGTGTGGACTTT |
| | | GCTGTTAATACAGAAGGCGTGTACTCTGAACCCCGCCCCATTGGCACCCGT |
| | | TACCTCACCCGTAATCTGTAA |
| AAV-DJ | 24 | MAADGYLPDWLEDTLSEGIRQWWKLKPGPPPPKPAERHKDDSRGLVLPGYK |
| (protein) | | YLGPFNGLDKGEPVNEADAAALEHDKAYDRQLDSGDNPYLKYNHADAEFQE |
| | | RLKEDTSFGGNLGRAVFQAKKRLLEPLGLVEEAAKTAPGKKRPVEHSPVEP |
| | | AGGGAPMADNNEGADGVGNSSGNWHCDSTWMGDRVITTSTRTWALPTYNNH |
| | | DSSSGTGKAGQQPARKRLNFGQTGDADSVPDPQPIGEPPAAPSGVGSLTMA |
| | | LYKQISNSTSGGSSNDNAYFGYSTPWGYFDFNRFHCHFSPRDWQRLINNNW |
| | | GFRPKRLSFKLFNIQVKEVTQNEGTKTIANNLTSTIQVFTDSEYQLPYVLG |
| | | SAHQGCLPPFPADVFMIPQYGYLTLNNGSQAVGRSSFYCLEYFPSQMLRTG |
| | | NNFQFTYTFEDVPFHSSYAHSQSLDRLMNPLIDQYLYYLSRTQTTGGTTNT |
| | | QTLGFSQGGPNTMANQAKNWLPGPCYRQQRVSKTSADNNNSEYSWTGATKY |
| | | HLNGRDSLVNPGPAMASHKDDEEKFFPQSGVLIFGKQGSEKTNVDIEKVMI |
| | | TDEEEIRTTNPVATEQYGSVSTNLQRGNRQAATADVNTQGVLPGMVWQDRD |
| | | VYLQGPIWAKIPHTDGHFHPSPLMGGFGLKHPPPQILIKNTPVPADPPTTF |
| | | NQSKLNSFITQYSTGQVSVEIEWELQKENSKRWNPEIQYTSNYYKSTSVDF |
| | | AVNTEGVYSEPRPIGTRYLTRNL |
| AAV- | 25 | ATGGCTGCCGATGGTTATCTTCCAGATTGGCTCGAGGACACTCTCTCTGAA |
| SL65 | | AACCAACAGCATCAGGACAACGGCAGGGGTCTTGTGCTTCCTGGGTACAAG |
| (nucleotide) | | GGCATTCGCGAGTGGTGGGCGCTGAAACCTGGAGCTCCACAACCCAAGGCC |
| | | TACCTCGGACCCTTCAACGGACTCGACAAGGGAGAGCCGGTCAACGAGGCA |
| | | GACGCCGCGGCCCTCGAGCACGACAAGGCCTACGACAAGCAGCTCGAGCAG |
| | | GGGGACAACCCGTACCTCAAGTACAACCACGCCGACGCCGAGTTTCAGGAG |
| | | CGTCTGCAAGAAGATACGTCTTTTGGGGGCAACCTCGGGCGAGCAGTCTTC |
| | | CAGGCCAAGAAGCGGGTTCTCGAACCTCTCGGTCTGGTTGAGGAAGGCGCT |
| | | AAGACGGCTCCTGGAAAGAAGAGACCGGTAGAGCCGTCACCTCAGCGTTCC |
| | | CCCGACTCCTCCACGGGCATCGGCAAGAAAGGCCAGCAGCCCGCCAGAAAG |
| | | AGACTCAATTTCGGTCAGACTGGCGACTCAGAGTCAGTCCCCGACCCTCAA |
| | | CCTCTCGGAGAACCTCCAGCAGCGCCCTCTAGTGTGGGATCTGGTACAGTG |
| | | GCTGCAGGCGGTGGCGCACCAATGGCAGACAATAACGAAGGTGCCGACGGA |
| | | GTGGGTAATGCCTCAGGAAATTGGCATTGCGATTCCACATGGCTGGGCGAC |
| | | AGAGTCATTACCACCAGCACCCGAACCTGGGCCCTGCCCACCTACAACAAC |
| | | CACCTCTACAAGCAAATCTCCAGCCAATCAGGAGCTTCAAACGACAACCAC |
| | | TACTTTGGCTACAGCACCCCTTGGGGGTATTTTGACTTTAACAGATTCCAC |
| | | TGCCACTTCTCACCACGTGACTGGCAGCGACTCATTAACAACAACTGGGGA |
| | | TTCCGGCCCAAGAGACTCAACTTCAAGCTCTTCAACATCCAAGTCAAGGAG |
| | | GTCACGACGAATGATGGCGTCACGACCATCGCTAATAACCTTACCAGCACG |
| | | GTTCAAGTCTTCTCGGACTCGGAGTACCAGTTGCCGTACGTCCTCGGCTCT |
| | | GCGCACCAGGGCTGCCTCCCTCCGTTCCCGGCGGACGTGTTCATGATTCCC |
| | | CAGTACGGCTACCTAACACTCAACAACGGTAGTCAGGCCGTGGGACGCTCC |
| | | TCCTTTTACTGCCTGGAATATTTCCCATCGCAGATGCTGAGAACGGGCAAT |
| | | AACTTTGAGTTCAGCTACAGCTTCGAGGACGTGCCTTTCCACAGCAGCTAC |
| | | GCACACAGCCAGAGCTTGGACCGACTGATGAATCCTCTCATTGACCAGTAC |
| | | CTGTACTACTTATCCAGAACTCAGTCCACAGGAGGAACTCAAGGTACCCAG |
| | | CAATTGTTATTTTCTCAAGCTGGGCCTGCAAACATGTCGGCTCAGGCCAAG |
| | | AACTGGCTGCCTGGACCTTGCTACCGGCAGCAGCGAGTCTCCACGACACTG |
| | | TCGCAAAACAACAACAGCAACTTTGCTTGGACTGGTGCCACCAAATATCAC |
| | | CTGAACGGCAGAAACTCGTTGGTTAATCCCGGCGTCGCCATGGCAACTCAC |
| | | AAGGACGACGAGGACCGCTTTTTCCCATCCAGCGGAGTCCTGATTTTTGGA |
| | | AAAACTGGAGCAACTAACAAAACTACATTGGAAAATGTGTTAATGACAAAT |
| | | GAAGAAGAAATTCGTCCTACTAATCCTGTAGCCACGGAAGAATACGGGATA |
| | | GTCAGCAGCAACTTACAAGCGGCTAATACTGCAGCCCAGACACAAGTTGTC |
| | | AACAACCAGGGAGCCTTACCTGGCATGGTCTGGCAGAACCGGGACGTGTAC |
| | | CTGCAGGGTCCCATTTGGGCCAAAATTCCTCACACAGATGGACACTTTCAC |
| | | CCGTCTCCTCTTATGGGCGGCTTTGGACTCAAGAACCCGCCTCCTCAGATC |
| | | CTCATCAAAAACACGCCTGTTCCTGCGAATCCTCCGGCGGAGTTTTCAGCT |
| | | ACAAAGTTTGCTTCATTCATCACCCAGTATTCCACAGGACAAGTGAGCGTG |
| | | GAGATTGAATGGGAGCTGCAGAAAGAAAACAGCAAACGCTGGAATCCCGAA |
| | | GTGCAGTATACATCTAACTATGCAAAATCTGCCAACGTTGATTTCACTGTG |
| | | GACAACAATGGACTTTATACTGAGCCTCGCCCCATTGGCACCCGTTACCTT |
| | | ACCCGTCCCCTGTAA |
| AAV- | 26 | MAADGYLPDWLEDTLSEGIREWWALKPGAPQPKANQQHQDNGRGLVLPGYK |
| SL65 | | YLGPFNGLDKGEPVNEADAAALEHDKAYDKQLEQGDNPYLKYNHADAEFQE |
| (protein) | | RLQEDTSFGGNLGRAVFQAKKRVLEPLGLVEEGAKTAPGKKRPVEPSPQRS |
| | | PDSSTGIGKKGQQPARKRLNFGQTGDSESVPDPQPLGEPPAAPSSVGSGTV |
| | | AAGGGAPMADNNEGADGVGNASGNWHCDSTWLGDRVITTSTRTWALPTYNN |
| | | HLYKQISSQSGASNDNHYFGYSTPWGYFDFNRFHCHFSPRDWQRLINNNWG |
| | | FRPKRLNFKLFNIQVKEVTTNDGVTTIANNLTSTVQVFSDSEYQLPYVLGS |
| | | AHQGCLPPFPADVFMIPQYGYLTLNNGSQAVGRSSFYCLEYFPSQMLRTGN |
| | | NFEFSYSFEDVPFHSSYAHSQSLDRLMNPLIDQYLYYLSRTQSTGGTQGTQ |
| | | QLLFSQAGPANMSAQAKNWLPGPCYRQQRVSTTLSQNNNSNFAWTGATKYH |
| | | LNGRNSLVNPGVAMATHKDDEDRFFPSSGVLIFGKTGATNKTTLENVLMTN |
| | | EEEIRPTNPVATEEYGIVSSNLQAANTAAQTQVVNNQGALPGMVWQNRDVY |
| | | LQGPIWAKIPHTDGHFHPSPLMGGFGLKNPPPQILIKNTPVPANPPAEFSA |
| | | TKFASFITQYSTGQVSVEIEWELQKENSKRWNPEVQYTSNYAKSANVDFTV |
| | | DNNGLYTEPRPIGTRYLTRPL |
| AAV- | 27 | ATGGCTGCCGATGGTTATCTTCCAGATTGGCTCGAGGACACTCTCTCTGAA |
| NP59 | | GGAATAAGACAGTGGTGGAAGCTCAAACCTGGCCCACCACCACCAAAGCCC |
| (nucleotide) | | GCAGAGCGGCATAAGGACGACAGCAGGGGTCTTGTGCTTCCTGGGTACAAG |
| | | TACCTCGGACCCTTCAACGGACTCGACAAGGGAGAGCCGGTCAACGAGGCA |
| | | GACGCCGCGGCCCTCGAGCACGACAAAGCCTACGACCGGCAGCTCGACAGC |
| | | GGAGACAACCCGTACCTCAAGTACAACCACGCCGACGCGGAGTTTCAGGAG |
| | | CGCCTTAAAGAAGATACGTCTTTTGGGGGCAACCTCGGACGAGCAGTCTTC |
| | | CAGGCGAAAAAGAGGGTTCTTGAACCTCTGGGCCTGGTTGAGGAACCTGTT |
| | | AAGACGGCTCCGGGAAAAAAGAGGCCGGTAGAGCACTCTCCTGTGGAGCCA |
| | | GACTCCTCCTCGGGAACCGGCAAGACAGGCCAGCAGCCCGCTAAAAAGAGA |
| | | CTCAATTTTGGTCAGACTGGCGACTCAGAGTCAGTCCCAGACCCTCAACCT |
| | | CTCGGAGAACCACCAGCAGCCCCCTCTGGTCTGGGAACTAATACGATGGCT |
| | | ACAGGCAGTGGCGCACCAATGGCAGACAATAACGAGGGTGCCGATGGAGTG |
| | | GGTAATTCCTCAGGAAATTGGCATTGCGATTCCCAATGGCTGGGCGACAGA |
| | | GTCATCACCACCAGCACCCGAACCTGGGCCCTGCCCACCTACAACAACCAT |
| | | CTCTACAAGCAAATCTCCAGCCAATCAGGAGCTTCAAACGACAACCACTAC |
| | | TTTGGCTACAGCACCCCTTGGGGGTATTTTGACTTTAACAGATTCCACTGC |
| | | CACTTCTCACCACGTGACTGGCAGCGACTCATTAACAACAACTGGGGATTC |
| | | CGGCCCAAGAAACTCAGCTTCAAGCTCTTCAACATCCAAGTTAAAGAGGTC |
| | | ACGCAGAACGATGGCACGACGACTATTGCCAATAACCTTACCAGCACGGTT |
| | | CAAGTGTTTACTGACTCGGAGTACCAGCTCCCGTACGTCCTCGGCTCGGCG |
| | | CATCAAGGATGCCTCCCGCCGTTCCCAGCAGACGTCTTCATGGTGCCACAG |
| | | TATGGATACCTCACCCTGAACAACGGGAGTCAGGCAGTAGGACGCTCTTCA |
| | | TTTTACTGCCTGGAGTACTTTCCTTCTCAGATGCTGCGTACCGGAAACAAC |
| | | TTTACCTTCAGCTACACTTTTGAGGACGTTCCTTTCCACAGCAGCTACGCT |
| | | CACAGCCAGAGTCTGGACCGTCTCATGAATCCTCTCATCGACCAGTACCTG |
| | | TATTACTTGAGCAGAACAAACACTCCAAGTGGAACCACCACGCAGTCAAGG |
| | | CTTCAGTTTTCTCAGGCCGGAGCGAGTGACATTCGGGACCAGTCTAGGAAC |
| | | TGGCTTCCTGGACCCTGTTACCGCCAGCAGCGAGTATCAAAGACATCTGCG |
| | | GATAACAACAACAGTGAATACTCGTGGGCTGGAGCTACCAAGTACCACCTC |
| | | AATGGCAGAGACTCTCTGGTGAATCCGGGCCCGGCCATGGCAAGCCACAAG |
| | | GACGATGAAGAAAAGTTTTTTCCTCAGAGCGGGGTTCTCATCTTTGGGAAG |
| | | CAAGGCTCAGAGAAAACAAATGTGGACATTGAAAAGGTCATGATTACAGAC |
| | | GAAGAGGAAATCAGGACAACCAATCCCGTGGCTACGGAGCAGTATGGTTCT |
| | | GTATCTACCAACCTCCAGAGAGGCAACAGACAAGCAGCTACCGCAGATGTC |
| | | GACACACAAGGCGTTCTTCCAGGCATGGTATGGCAGGACAGAGATGTGTAC |
| | | CTTCAGGGACCCATCTGGGCAAAGATTCCACACACGGACGGACATTTTCAC |
| | | CCCTCTCCCCTCATGGGTGGATTCGGACTTAAACACCCTCCTCCACAGATT |
| | | CTCATCAAGAACACCCCGGTACCTGCGAATCCTTCGACCACCTTCAGTGCG |
| | | GCAAAGTTTGCTTCCTTCATCACACAGTACTCAACGGGACAGGTCAGCGTG |
| | | GAGATCGAGTGGGAGCTGCAGAAGGAAAACAGCAAACGCTGGAATCCCGAA |
| | | ATTCAGTACACTTCCAACTACAACAAGTCTGTTAATGTGGACTTTACTGTG |
| | | GACACTAATGGCGTGTATTCAGAGCCTCGCCCCATTGGCACCAGATACCTG |
| | | ACTCGTAATCTGTAA |
| AAV- | 28 | MAADGYLPDWLEDTLSEGIRQWWKLKPGPPPPKPAERHKDDSRGLVLPGYK |
| NP59 | | YLGPFNGLDKGEPVNEADAAALEHDKAYDRQLDSGDNPYLKYNHADAEFQE |
| (protein) | | RLKEDTSFGGNLGRAVFQAKKRVLEPLGLVEEPVKTAPGKKRPVEHSPVEP |
| | | DSSSGTGKTGQQPAKKRLNFGQTGDSESVPDPQPLGEPPAAPSGLGTNTMA |
| | | TGSGAPMADNNEGADGVGNSSGNWHCDSQWLGDRVITTSTRTWALPTYNNH |
| | | LYKQISSQSGASNDNHYFGYSTPWGYFDFNRFHCHFSPRDWQRLINNNWGF |
| | | RPKKLSFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FTFSYTFEDVPFHSSYAHSQSLDRLMNPLIDQYLYYLSRTNTPSGTTTQSR |
| | | LQFSQAGASDIRDQSRNWLPGPCYRQQRVSKTSADNNNSEYSWAGATKYHL |
| | | NGRDSLVNPGPAMASHKDDEEKFFPQSGVLIFGKQGSEKTNVDIEKVMITD |
| | | EEEIRTTNPVATEQYGSVSTNLQRGNRQAATADVDTQGVLPGMVWQDRDVY |
| | | LQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTV |
| | | DTNGVYSEPRPIGTRYLTRNL |

In some embodiments, a recombinant AAV vector comprises at least one ITR. In some embodiments, a recombinant AAV vector comprises two ITRs. In some embodiments, a recombinant AAV vector comprises a 5′ ITR. In some embodiments, a recombinant AAV vector comprises a 3′ ITR. In some embodiments, a recombinant AAV vector comprises an AAV2 ITR. In some embodiments, a recombinant AAV vector comprises a portion of an AAV2 ITR. In some embodiments, a recombinant AAV vector comprises an ITR having at least 80%, 85%, 90%, 95%, 99%, or 100% sequence identity to an AAV2 ITR. In some embodiments, a recombinant AAV vector comprises an ITR having 90%, 95%, 99%, 100% sequence identity to one of SEQ ID Nos. 29-32.

145 bp ITR:
(SEQ ID NO. 29)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGAGTGGCCAA
130 bp ITR:
(SEQ ID NO. 30)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG
B loop deletion ITR:
(SEQ ID NO. 31)
AGGAACCCCTAGTGATGGAGTTGGCCACTCCCTCTCTGCGCGCTCGCTC
GCTCACTGAGGCCGCCCGGGCTTTGCCCGGGCGGCCTCAGTGAGCGAGC
GAGCGCGCAGAGAGGGAGTGGCCAA
C loop deletion ITR:
(SEQ ID NO. 32)
AGGAACCCCTAGTGATGGAGTTGGCCACTCCCTCTCTGCGCGCTCGCTC
GCTCACTGAGGCCGGGCGACCAAAGGTCGCCCGGCCTCAGTGAGCGAGC
GAGCGCGCAGAGAGGGAGTGGCCAA

### Methods of Treatment

Compositions and constructs disclosed herein may be used in any in vitro or in vivo application wherein expression of a payload (e.g. transgene) from a particular target locus in a cell, while maintaining expression of endogenous genes at and surrounding the target locus, is desired. For example, compositions and constructs disclosed herein may be used to treat a disorder, disease, or medical condition in a subject (e.g., through gene therapy).

In some embodiments, treatment comprises obtaining or maintaining a desired pharmacologic and/or physiologic effect. In some embodiments, a desired pharmacologic and/or physiologic effect may comprise completely or partially preventing a disease (e.g., preventing symptoms of disease). In some embodiments, a desired pharmacologic and/or physiologic effect may comprise completely or partially curing a disease (e.g., curing adverse effects associated with a disease). In some embodiments, a desired pharmacologic and/or physiologic effect may comprise preventing recurrence of a disease. In some embodiments, a desired pharmacologic and/or physiologic effect may comprise slowing progression of a disease. In some embodiments, a desired pharmacologic and/or physiologic effect may comprise relieving symptoms of a disease. In some embodiments, a desired pharmacologic and/or physiologic effect may comprise preventing regression of a disease. In some embodiments, a desired pharmacologic and/or physiologic effect may comprise stabilizing and/or reducing symptoms associated with a disease.

In some embodiments, treatment comprises administering a composition before, during, or after onset of a disease (e.g., before, during, or after appearance of symptoms associated with a disease). In some embodiments, treatment comprises combination therapy (e.g., with one or more therapies, including different types of therapies).

### Targeted Integration

In some embodiments, compositions and constructs provided herein direct integration of a payload (e.g., a transgene and/or functional nucleic acid) at a target locus (also referred to herein as a target integration site) (e.g., an endogenous gene). In some embodiments, compositions and constructs provided herein direct integration of a payload at a target locus in a specific cell type (e.g., tissue-specific loci). In some embodiments, integration of a payload occurs in a specific tissue (e.g., liver, central nervous system (CNS), muscle, kidney, vascular. lung). In some embodiments, integration of a payload occurs in multiple tissues (e.g., liver, central nervous system (CNS), muscle, kidney, vascular, lung).

In some embodiments, compositions and constructs provided herein direct integration of a payload at a target locus that is considered a safe-harbor site (e.g., albumin, Apolipoprotein A2 (ApoA2), Haptoglobin). In some embodiments, a target locus may be selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, integration of a payload occurs at a 5′ or 3′ end of one or more endogenous genes (e.g., genes encoding polypeptides). In some embodiments, integration of a payload occurs between a 5′ or 3′ end of one or more endogenous genes (e.g., genes encoding polypeptides).

In some embodiments, compositions and constructs provided herein direct integration of a payload at a target locus with minimal or no off-target integration (e.g., integration at a non-target locus). In some embodiments, compositions and constructs provided herein direct integration of a payload at a target locus with reduced off-target integration compared to a reference composition or construct (e.g., relative to a composition or construct without flanking homology sequences).

In some embodiments, integration of a transgene at a target locus allows expression of a payload without disrupting endogenous gene expression. In some embodiments, integration of a transgene at a target locus allows expression of a payload from an endogenous promoter. In some embodiments, integration of a transgene at a target locus disrupts endogenous gene expression. In some embodiments, integration of a transgene at a target locus disrupts endogenous gene expression without adversely affecting a target cell and/or subject (e.g., by targeting a safe-harbor site). In some embodiments, integration of a transgene at a target locus does not require use of a nuclease (e.g., Cas proteins, endonucleases, TALENs, ZFNs). In some embodiments, integration of a transgene at a target locus is assisted by use of a nuclease (e.g., Cas proteins, endonucleases, TALENs, ZFNs).

In some embodiments, integration of a transgene at a target locus confers a selective advantage (e.g., increased survival rate in a plurality of cells relative to other cells in a tissue). In some embodiments, a selective advantage may produce an increased percentage of cells in one or more tissues expressing a transgene.

### Compositions

In some embodiments, compositions can be produced using methods and constructs provided herein (e.g., viral vectors). In some embodiments, compositions include liquid, solid, and gaseous compositions. In some embodiments, compositions comprise additional ingredients (e.g., diluents, stabilizer, excipients, adjuvants). In some embodiments, additional ingredients can comprise buffers (e.g., phosphate, citrate, organic acid buffers), antioxidants (e.g., ascorbic acid), low molecular weight polypeptides (e.g., less than 10 residues), various proteins (e.g., serum albumin, gelatin, immunoglobulins), hydrophilic polymers (e.g., polyvinylpyrrolidone), amino acids (e.g., glycine, glutamine, asparagine, arginine, lysine), carbohydrates (e.g., monosaccharides, disaccharides, glucose, mannose, dextrins), chelating agents (e.g., EDTA), sugar alcohols (e.g., mannitol, sorbitol), salt-forming counterions (e.g., sodium, potassium), and/or nonionic surfactants (e.g. Tween™, Pluronics™, polyethylene glycol (PEG)), among other things. In some embodiments, an aqueous carrier is an aqueous pH buffered solution.

In some embodiments, compositions provided herein may be provided in a range of dosages. In some embodiments, compositions provided herein may be provided in a single dose. In some embodiments, compositions provided herein may be provided in multiple dosages. In some embodiments, compositions are provided over a period of time. In some embodiments, compositions are provided at specific intervals (e.g., varying intervals, set intervals). In some embodiments, dosages may vary depending upon dosage form and route of administration. In some embodiments, compositions provided herein may be provided in dosages between 1E12 and 1E14 vg/kg. In some embodiments, compositions provided herein may be provided in dosages between 3E12 and 1E13 vg/kg. In some embodiments, compositions provided herein may be provided in dosages between 1E13 and 3E13 vg/kg. In some embodiments, compositions provided herein may be provided in dosages between 3E12 and 3E13 vg/kg. In some embodiments, compositions provided herein may be provided in dosages of no more than 3E13 vg/kg. In some embodiments, compositions provided herein may be provided in dosages of no more than 1E13 vg/kg. In some embodiments, compositions provided herein may be provided in dosages of no more than 3E12 vg/kg.

In some embodiments, compositions provided herein may be administered to a subject at a particular timepoint (e.g., age of a subject). In some embodiments, compositions provided herein may be administered to a newborn subject. In some embodiments, compositions provided herein may be administered to a neonatal subject. In some embodiments, a neonatal mouse subject is between 0 and 14 days of age. In some embodiments, a neonatal human subject is between 0 days and 1 month of age. In some embodiments compositions provided herein may be administered to a subject between 7 days of age and 30 days of age. In some embodiments, compositions provided herein may be administered to a subject between 3 months of age and 1 year of age. In some embodiments, compositions provided herein may be administered to a subject between 1 year of age and 5 years of age. In some embodiments, compositions provided herein may be administered to a subject between 4 years of age and 7 years of age. In some embodiments, compositions provided herein may be administered to a subject at 5 years of age or older.

In some embodiments, compositions provided herein may be administered to a subject at a particular timepoint based upon growth stage (e.g., percentage of estimated/average adult size or weight) of a particular tissue or organ. In some embodiments, compositions provided herein may be administered to a subject wherein a tissue or organ (e.g., liver, muscle, CNS, lung, etc.) is at least 10%, at least 20%, at least 30%, at least 40%, at least 50%, at least 60%, at least 70%, at least 80%, at least 90%, or at least 99% of estimated/average adult size or weight. In some embodiments, compositions provided herein may be administered to a subject wherein a tissue or organ is approximately 20% (+/−5%) of estimated/average adult size or weight. In some embodiments, compositions provided herein may be administered to a subject wherein a tissue or organ is approximately 50% (+/−5%) of estimated/average adult size or weight. In some embodiments, compositions provided herein may be administered to a subject wherein a tissue or organ is approximately 60% (+/−5%) of estimated/average adult size or weight. In some embodiments, estimated/average adult size or weight of a particular tissue or organ may be determined as described in the art (See, Noda et al. Pediatric radiology, 1997; Johnson et al. Liver transplantation, 2005; and Szpinda et al. Biomed research international, 2015, each of which is incorporated herein by reference in its entirety.

### Routes of Administration

In some embodiments, compositions provided herein may be administered to a subject via any one (or more) of a variety of routes known in the art (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, compositions provided herein may be introduced into cells, which are then introduced into a subject (e.g., liver, muscle, central nervous system (CNS), lung, hematologic cells). In some embodiments, compositions provided herein may be introduced via delivery methods known in the art (e.g., injection, catheter).

In some embodiments, genome editing with the GENERIDE™ platform differs from conventional gene therapy because it uses homologous recombination to deliver a corrective gene to one specific location in the genome. In some embodiments, GENERIDE™ inserts a corrective gene in a precise manner, leading to site-specific integration in the genome. In some embodiments, GENERIDE™ does not require the use of exogenous nucleases or promoters. In some embodiments, GENERIDE™ may be combined with one or more exogenous nucleases and/or promoters.

In some embodiments, provided compositions comprise one or more homology arms, a transgene, and a nucleic acid that promotes the production of two independent gene products. In some embodiments, compositions and methods of the present disclosure comprise a first nucleic acid sequence encoding a transgene. In some embodiments, compositions and methods of the present disclosure comprise a second nucleic acid that promotes the production of two independent gene products (e.g., a 2A peptide). In some embodiments, the present disclosure provides and expression cassette comprising a first nucleic acid sequence and a second nucleic acid sequence as described herein.

In some embodiments, a second nucleic acid comprises a nucleic acid sequence encoding a 2A peptide; a nucleic acid sequence encoding an internal ribosome entry site (IRES); a nucleic acid sequence encoding an N-terminal intein splicing region and C-terminal intein splicing region; and/or a nucleic acid sequence encoding a splice donor and a splice acceptor. In some embodiments, compositions and methods of the present disclosure comprise a polynucleotide cassette comprising an expression cassette comprising said first nucleic acid and said second nucleic acid. In some embodiments, compositions and methods of the present disclosure comprise a third nucleic acid sequence comprising a sequence that is substantially homologous to a genomic sequence. In some embodiments, compositions and methods of the present disclosure comprise a fourth nucleic acid sequence comprising a sequence that is substantially homologous to a genomic sequence. In some embodiments, said third nucleic acid sequence is positioned 5′ to the expression cassette and comprises a sequence that is substantially homologous to a genomic sequence 5′ of a target integration site in a genome of a cell. In some embodiments, said fourth nucleic acid sequence is positioned 3′ to the expression cassette and comprises a sequence that is substantially homologous to a genomic sequence 3′ of a target integration site in the genome of the cell.

In some embodiments, one or more compositions described herein are administered without any additional treatment. In some embodiments, one or more compositions described herein are administered in combination. In some embodiments, a first composition may be administered simultaneously with a second composition. In some embodiments, a first composition and second composition may be administered sequentially (e.g., within minutes, hours, days, weeks, or months of one another). In some embodiments, one or more compositions may be administered via the same route (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, one or more compositions may be administered via different routes (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal).

In some embodiments, the first and/or second compositions are administered at a particular dose (e.g., a fixed dose or a weight based dose) only once. In some embodiments, the first and/or second compositions are administered at a particular dose (e.g., a fixed dose or a weight based dose) more than once. In some embodiments, where more than one dose is administered (e.g., a fixed dose or a weight based dose) the first and/or second compositions may be administered simultaneously, substantially simultaneously, or consecutively. In some embodiments, multiple doses (e.g., a fixed dose or a weight based dose) are administered within a specified period of time (e.g., within minutes, hours, days, weeks, or months).

In some embodiments, the first and/or second compositions are administered in response to a biomarker (e.g., a circulating biomarker as described in WO2020214582A1). For example, the first and/or second compositions are administered at a particular dose (e.g., a fixed dose or a weight based dose) and within a specified period of time (e.g., within minutes, hours, days, weeks, or months) levels of a biomarker (e.g., as described in WO2020214582A1) are monitored. If levels of a biomarker (e.g., as described in WO2020214582A1) are low (e.g., as compared to an appropriate reference (e.g., levels of a biomarker prior to administration)), then the first and/or second compositions are administered at a particular dose (e.g., a fixed dose or a weight based dose). If levels of a biomarker (e.g., as described in WO2020214582A1) are high (e.g., as compared to an appropriate reference (e.g., levels of a biomarker after an initial administration)), then subsequent dosing (e.g., a fixed dose or a weight based dose) of the first and/or second compositions may be reevaluated (e.g., treatment suspension, reduced fixed dose or weight based dose).

### Methods of Producing Viral Vectors

### Production of Viral Vectors

In some embodiments, production of viral vectors (e.g., AAV viral vectors) may include both upstream steps to generate viral vectors (e.g. cell-based culturing) and downstream steps to process viral vectors (e.g., purification, formulation, etc.). In some embodiments, upstream steps may comprise one or more of cell expansion, cell culture, cell transfection, cell lysis, viral vector production, and/or viral vector harvest.

In some embodiments, downstream steps may comprise one or more of separation, filtration, concentration, clarification, purification, chromatography (e.g., affinity, ion exchange, hydrophobic, mixed-mode), centrifugation (e.g., ultracentrifugation), and/or formulation.

In some embodiments, constructs and methods described herein are designed to increase viral vector yields (e.g., AAV vector yields), reduce levels of replication-competent viral vectors (e.g., replication competent AAV (rcAAV)), improve viral vectors packaging efficiency (e.g., AAV vector capsid packaging), and/or any combinations thereof, relative to a reference construct or method, for example those in Xiao et al. 1998 and Grieger et al. 2015, each of which is incorporated herein by reference in its entirety.

### Cell Lines and Transfection Reagents

In some embodiments, production of viral vectors comprises use of cells (e.g., cell culture). In some embodiments, production of viral vectors comprises use of cell culture comprising one or more cell lines (e.g., mammalian cell lines). In some embodiments, production of viral vectors comprises use of HEK293 cell lines or variants thereof (e.g., HEK293T, HEK293F cell lines). In some embodiments, cells are capable of being grown in suspension. In some embodiments, cells are comprised of adherent cells. In some embodiments, cells are capable of being grown in media that does not comprise animal components (e.g. animal serum). In some embodiments, cells are capable of being grown in serum-free media (e.g., F17 media, Expi293 media). In some embodiments, production of viral vectors comprises transfection of cells with expression constructs (e.g., plasmids). In some embodiments, cells are selected for high expression of viral vectors (e.g. AAV vectors). In some embodiments, cells are selected for high packaging efficiency of viral vectors (e.g., capsid packaging of AAV vectors). In some embodiments, cells are selected for improved transfection efficiency (e.g., with chemical transfection reagents, including cationic molecules). In some embodiments, cells are engineered for high expression of viral vectors (e.g. AAV vectors). In some embodiments, cells are engineered for high packaging efficiency of viral vectors (e.g., capsid packaging of AAV vectors). In some embodiments, cells are engineered for improved transfection efficiency (e.g., with chemical transfection reagents, including cationic molecules). In some embodiments, cells may be engineered or selected for two or more of the above attributes. In some embodiments, cells are contacted with one or more expression constructs (e.g. plasmids). In some embodiments, cells are contacted with one or more transfection reagents (e.g., chemical transfection reagents, including lipids, polymers, and cationic molecules) and one or more expression constructs. In some embodiments, cells are contacted with one or more cationic molecules (e.g., cationic lipid, PEI reagent) and one or more expression constructs. In some embodiments, cells are contacted with a PEIMAX reagent and one or more expression constructs. In some embodiments, cells are contacted with a FectoVir-AAV reagent and one or more expression constructs. In some embodiments, cells are contacted with one or more transfection reagents and one or more expression constructs at particular ratios. In some embodiments, ratios of transfection reagents to expression constructs improves production of viral vectors (e.g., improved vector yield, improved packaging efficiency, and/or improved transfection efficiency).

### Expression Constructs

In some embodiments, expression constructs are or comprise one or more polynucleotide sequences (e.g., plasmids). In some embodiments, expression constructs comprise particular polynucleotide sequence elements (e.g., payloads, promoters, viral genes, etc.). In some embodiments, expression constructs comprise polynucleotide sequences encoding viral genes (e.g., a rep or cap gene or gene variant, one or more helper virus genes or gene variants). In some embodiments, expression constructs of a particular type comprise specific combinations of polynucleotide sequence elements. In some embodiments, expression constructs of a particular type do not comprise specific combinations of polynucleotide sequence elements. In some embodiments, a particular expression construct does not comprise polynucleotide sequence elements encoding both rep and cap genes and/or gene variants.

In some embodiments, expression constructs comprise polynucleotide sequences encoding wild-type viral genes (e.g., wild-type rep genes, cap genes, viral helper genes, or combinations thereof). In some embodiments, expression constructs comprise polynucleotide sequences encoding viral helper genes or gene variants (e.g., herpesvirus genes or gene variants, adenovirus genes or gene variants). In some embodiments, expression constructs comprise polynucleotide sequences encoding one or more gene copies that express one or more wild-type Rep proteins (e.g., 1 copy, 2 copies, 3 copies, 4 copies, 5 copies, etc.). In some embodiments, expression constructs comprise polynucleotide sequences encoding a single gene copy that expresses one or more wild-type Rep proteins (e.g., Rep68, Rep40, Rep52, Rep78, or combinations thereof). In some embodiments, expression constructs comprise polynucleotide sequences encoding one or more wild-type Rep proteins (e.g., Rep68, Rep40, Rep52, Rep78, or combinations thereof). In some embodiments, expression constructs comprise polynucleotide sequences encoding at least four wild-type Rep proteins (e.g., Rep68, Rep40, Rep52, Rep78). In some embodiments, expression constructs comprise polynucleotide sequences encoding each of Rep68, Rep40, Rep52, and Rep78. In some embodiments, expression constructs comprise polynucleotide sequences encoding one or more wild-type adenoviral helper proteins (e.g., E2 and E4).

In some embodiments, expression constructs comprise wild-type polynucleotide sequences encoding wild-type viral genes (e.g., rep genes, cap genes, helper genes). In some embodiments, expression constructs comprise modified polynucleotide sequences (e.g., codon-optimized) encoding wild-type viral genes (e.g., rep genes, cap genes, helper genes). In some embodiments, expression constructs comprise modified polynucleotide sequences encoding modified viral genes (e.g., rep genes, cap genes, helper genes). In some embodiments, modified viral genes are designed and/or engineered for certain improvements (e.g., improved transduction, tissue specificity, reduced size, reduced immune response, improved packaging, reduced rcAAV levels, etc.).

In accordance with various embodiments, expression constructs disclosed herein may offer increased flexibility and modularity as compared to previous technologies. In some embodiments, expression constructs disclosed herein may allow swapping of various polynucleotide sequences (e.g., different rep genes, cap genes, payloads, helper genes, promoters, etc.) while providing certain improvements (e.g., increased viral vector yield, increased packaging, reduced rcAAV levels, etc.). In some embodiments, expression constructs disclosed herein are compatible with various upstream production processes (e.g., different cell culture conditions, different transfection reagents, etc.) while providing certain improvements (e.g., increased viral vector yield, increased packaging, reduced rcAAV levels, etc.)

In some embodiments, expression constructs of different types comprise different combinations of polynucleotide sequences. In some embodiments, an expression construct of one type comprises one or more polynucleotide sequence elements (e.g., payloads, promoters, viral genes, etc.) that is not present in an expression construct of a different type. In some embodiments, an expression construct of one type comprises polynucleotide sequence elements encoding a viral gene (e.g., a rep or cap gene or gene variant) and polynucleotide sequence elements encoding a payload (e.g., a transgene and/or functional nucleic acid). In some embodiments, an expression construct of one type comprises polynucleotide sequence elements encoding one or more viral genes (e.g., a rep or cap gene or gene variant and/or one or more helper virus genes). In some embodiments, an expression construct of one type comprises polynucleotide sequence elements encoding one or more viral genes, wherein the viral genes are from one or more virus types (e.g., genes or gene variants from AAV and adenovirus). In some embodiments, viral genes from adenovirus are genes and/or gene variants. In some embodiments, viral genes from adenovirus are one or more of E2A (e.g., E2A DNA Binding Protein (DBP), E4 (e.g., E4 Open Reading Frame (ORF) 2, ORF3, ORF4, ORF6/7), VA, and/or variants thereof. In some embodiments, expression constructs are used for production of viral vectors (e.g. through cell culture). In some embodiments, expression constructs are contacted with cells in combination with one or more transfection reagents (e.g., chemical transfection reagents). In some embodiments, expression constructs are contacted with cells at particular ratios in combination with one or more transfection reagents. In some embodiments, expression constructs of different types are contacted with cells at particular ratios (e.g., weight ratios) in combination with one or more transfection reagents. In some embodiments, expression constructs of different types are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio). In some embodiments, a first expression construct comprising one or more viral helper genes and a second expression construct comprising one or more payloads are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, a first expression construct comprising one or more payloads and a second expression construct comprising one or more viral helper genes are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, particular ratios of expression constructs improve production of AAV (e.g., increased viral vector yields, increased packaging efficiency, and/or increased transfection efficiency. In some embodiments, cells are contacted with two or more expression constructs (e.g., sequentially or substantially simultaneously). In some embodiments, three or more expression constructs are contacted with cells. In some embodiments, expression constructs comprise one or more promoters (e.g., one or more exogenous promoters). In some embodiments, promoters are or comprise CMV, RSV, CAG, EF1alpha, PGK, A1AT, C5-12, MCK, desmin, p5, p40, or combinations thereof. In some embodiments, expression constructs comprise one or more promoters upstream of a particular polynucleotide sequence element (e.g., a rep or cap gene or gene variant). In some embodiments, expression constructs comprise one or more promoters downstream of a particular polynucleotide sequence element (e.g., a rep or cap gene or gene variant).

In some embodiments, a first expression construct comprising one or more viral helper genes and a second expression construct comprising one or more payloads are contacted with cells at a ratio greater than or equal to 1:1 up to 3:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 5:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 6:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 8:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload). In some embodiments, a first expression construct comprising one or more viral helper genes and a second expression construct comprising one or more payloads are contacted with cells at a ratio greater than or equal to 1:1 up to 10:1, wherein viral titer yields are at at least 1.5× greater than those obtained through administration of a reference system (e.g., a three-plasmid comprising separate plasmids, each encoding one of: 1) an AAV rep and AAV cap sequence, 2) relevant sequence from a helper virus, and 3) a payload).

In some embodiments, a first expression construct comprising one or more viral helper genes and a second expression construct comprising one or more payloads are contacted with cells at a ratio between 10:1 and 1:1. In some embodiments, a first expression construct comprising one or more viral helper genes and a second expression construct comprising one or more payloads are contacted with cells at a ratio between 9:1 and 1:1. In some embodiments, a first expression construct comprising one or more viral helper genes and a second expression construct comprising one or more payloads are contacted with cells at a ratio between 8:1 and 1:1. In some embodiments, a first expression construct comprising one or more viral helper genes and a second expression construct comprising one or more payloads are contacted with cells at a ratio between 7:1 and 1:1. In some embodiments, a first expression construct comprising one or more viral helper genes and a second expression construct comprising one or more payloads are contacted with cells at a ratio between 6:1 and 1:1. In some embodiments, a first expression construct comprising one or more viral helper genes and a second expression construct comprising one or more payloads are contacted with cells at a ratio between 5:1 and 1:1. In some embodiments, a first expression construct comprising one or more viral helper genes and a second expression construct comprising one or more payloads are contacted with cells at a ratio between 4:1 and 1:1. In some embodiments, a first expression construct comprising one or more viral helper genes and a second expression construct comprising one or more payloads are contacted with cells at a ratio between 3:1 and 1:1. In some embodiments, a first expression construct comprising one or more viral helper genes and a second expression construct comprising one or more payloads are contacted with cells at a ratio between 2:1 and 1:1.

In some embodiments, a first expression construct comprising one or more viral helper genes and a second expression construct comprising one or more payloads are contacted with cells at a ratio between 1:1 and 2:1. In some embodiments, a first expression construct comprising one or more viral helper genes and a second expression construct comprising one or more payloads are contacted with cells at a ratio between 1:1 and 3:1. In some embodiments, a first expression construct comprising one or more viral helper genes and a second expression construct comprising one or more payloads are contacted with cells at a ratio between 1:1 and 4:1. In some embodiments, a first expression construct comprising one or more viral helper genes and a second expression construct comprising one or more payloads are contacted with cells at a ratio between 1:1 and 5:1. In some embodiments, a first expression construct comprising one or more viral helper genes and a second expression construct comprising one or more payloads are contacted with cells at a ratio between 1:1 and 6:1. In some embodiments, a first expression construct comprising one or more viral helper genes and a second expression construct comprising one or more payloads are contacted with cells at a ratio between 1:1 and 7:1. In some embodiments, a first expression construct comprising one or more viral helper genes and a second expression construct comprising one or more payloads are contacted with cells at a ratio between 1:1 and 8:1. In some embodiments, a first expression construct comprising one or more viral helper genes and a second expression construct comprising one or more payloads are contacted with cells at a ratio between 1:1 and 9:1. In some embodiments, a first expression construct comprising one or more viral helper genes and a second expression construct comprising one or more payloads are contacted with cells at a ratio between 1:1 and 10:1. In some embodiments, a first expression construct comprising one or more viral helper genes and a second expression construct comprising one or more payloads are contacted with cells at a ratio of 1.5:1.

In some embodiments, expression constructs comprise one or more polynucleotide sequences encoding elements (e.g., selection markers, origins of replication) necessary for cell culture (e.g., bacterial cell culture, mammalian cell culture). In some embodiments, expression constructs comprise one or more polynucleotide sequences encoding antibiotic resistance genes (e.g., kanamycin resistance genes, ampicillin resistance genes). In some embodiments, expression constructs comprise one or more polynucleotide sequences encoding a bacterial original of replication (e.g., colE1 origin of replication).

In some embodiments, expression constructs comprise one or more transcription termination sequences (e.g., a polyA sequence). In some embodiments, expression constructs comprise one or more of BGH polyA, FIX polyA, SV40 polyA, synthetic polyA, or combinations thereof. In some embodiments, expression constructs comprise one or more transcription termination sequences downstream of a particular sequence element (e.g., a rep or cap gene or gene variant). In some embodiments, expression constructs comprise one or more transcription termination sequences upstream of a particular sequence element (e.g., a rep or cap gene or gene variant).

In some embodiments, expression constructs comprise one or more intron sequences. In some embodiments, expression constructs comprise one or more of introns of different origins (e.g., known genes), including but not limited to FIX intron, Albumin intron, or combinations thereof. In some embodiments, expression constructs comprise one or more introns of different lengths (e.g., 133 bp to 4 kb). In some embodiments, expression constructs comprise one or more intron sequences upstream of a particular sequence element (e.g., a rep or cap gene or gene variant). In some embodiments, expression constructs comprise one or more intron sequences within a particular sequence element (e.g., a rep or cap gene or gene variant). In some embodiments, expression constructs comprise one or more intron sequences downstream of particular sequence element (e.g., a rep or cap gene or gene variant). In some embodiments, expression constructs comprise one or more intron sequences after a promoter (e.g., a p5 promoter). In some embodiments, expression constructs comprise one or more intron sequences before a rep gene or gene variant. In some embodiments, expression constructs comprise one or more intron sequences between a promoter and a rep gene or gene variant. In some embodiments, compositions provided herein comprise expression constructs. In some embodiments, compositions comprise: (i) a first expression construct comprising a polynucleotide sequence encoding one or more rep genes and a polynucleotide sequence encoding one or more wild-type adenoviral helper proteins; and (ii) a second expression construct comprising a polynucleotide sequence encoding one or more cap genes and one or more payloads.

In some embodiments, expression constructs will comprise a three-plasmid (e.g., triple transfection) system for production of viral vectors. In some embodiments, a three-plasmid system will comprise: 1) a first plasmid comprising one or more sequences encoding a rep and cap gene, or variant thereof; 2) a second sequence encoding one or more payloads; and 3) a third sequence encoding one or more helper proteins. In some embodiments, a three-plasmid system may be used to produce one or more viral vectors disclosed herein.

### Methods of Characterizing AAV Viral Vectors

In accordance with various embodiments, viral vectors may be characterized through assessment of various characteristics and/or features. In some embodiments, assessment of viral vectors can be conducted at various points in a production process. In some embodiments, assessment of viral vectors can be conducted after completion of upstream production steps. In some embodiments, assessment of viral vectors can be conducted after completion of downstream production steps.

### Viral Yields

In some embodiments, characterization of viral vectors comprises assessment of viral yields (e.g., viral titer). In some embodiments, characterization of viral vectors comprises assessment of viral yields prior to purification and/or filtration. In some embodiments, characterization of viral vectors comprises assessment of viral yields after purification and/or filtration. In some embodiments, characterization of viral vectors comprises assessing whether viral yield is greater than or equal to 1e10 vg/mL.

In some embodiments, characterization of viral vectors comprises assessing whether viral yield in crude cell lysates is greater than or equal to 1e11 vg/mL. In some embodiments, characterization of viral vectors comprises assessing whether viral yield in crude cell lysates is greater than or equal to 5e11 vg/mL. In some embodiments, characterization of viral vectors comprises assessing whether viral yield in crude cell lysates is greater than or equal to 1e12 vg/mL. In some embodiments, characterization of viral vectors comprises assessing whether viral yield in crude lysates is between 5e9 vg/mL and 5e11 vg/mL. In some embodiments, characterization of viral vectors comprises assessing whether viral yield in crude lysates is between 5e9 vg/mL and 1e10 vg/mL. In some embodiments, characterization of viral vectors comprises assessing whether viral yield in crude lysates is between 1e10 vg/mL and 1e11 vg/mL. In some embodiments, characterization of viral vectors comprises assessing whether viral yield in crude lysates is between 1e11 vg/mL and 1e12 vg/mL. In some embodiments, characterization of viral vectors comprises assessing whether viral yield in crude lysates is between 1e12 vg/mL and 1e13 vg/mL.

In some embodiments, characterization of viral vectors comprises assessing whether viral yield in purified drug product is greater than or equal to 1e1 vg/mL. In some embodiments, characterization of viral vectors comprises assessing whether viral yield in purified drug product is greater than or equal to 1e12 vg/mL. In some embodiments, characterization of viral vectors comprises assessing whether viral yield in purified drug product is between 1e10 vg/mL and 1e15 vg/mL. In some embodiments, characterization of viral vectors comprises assessing whether viral yield in purified drug product is between 1e11 vg/mL and 1e15 vg/mL. In some embodiments, characterization of viral vectors comprises assessing whether viral yield in purified drug product is between 1e12 vg/mL and 1e14 vg/mL. In some embodiments, characterization of viral vectors comprises assessing whether viral yield in purified drug product is between 1e13 and 1e14 vg/mL.

In some embodiments, methods and compositions provided herein can provide comparable or increased viral vector yields as compared to previous methods known in the art. For example, in some embodiments, provided methods for producing and/or manufacturing viral vectors comprising use of a two-plasmid transfection system provide comparable or increased viral vector yields as compared to a three-plasmid system. In some embodiments, provided methods for producing and/or manufacturing viral vectors comprising use of a two-plasmid transfection system with particular combinations of sequence elements provide comparable or increased viral vector yields as compared to a two-plasmid system with a different combination of sequence elements. In some embodiments, provided methods for producing and/or manufacturing viral vectors comprising use of a two-plasmid transfection system with particular plasmid ratios provide comparable or increased viral vector yields as compared to a two-plasmid system with different plasmid ratios. In some embodiments, provided methods for producing and/or manufacturing viral vectors comprising use of a two-plasmid transfection system with particular plasmid ratios provide comparable or increased viral vector yields as compared to a reference (e.g., two-plasmid system with different plasmid ratios, three-plasmid system) under particular culture conditions. In some embodiments, provided methods for producing and/or manufacturing viral vectors comprising use of a two-plasmid transfection system with particular plasmid ratios provide comparable or increased viral vector yields as compared to a reference (e.g., two-plasmid system with different plasmid ratios, three-plasmid system) under large-scale culture conditions (e.g., greater than 100 mL, greater than 250 mL, greater than 1 L, greater than 10 L, greater than 20 L, greater than 30 L, greater than 40 L, greater than 50 L, etc.).

### Viral Packaging

In some embodiments, characterization of viral vectors comprises assessment of viral packaging efficiency (e.g., percent of full versus empty capsids). In some embodiments, characterization of viral vectors comprises assessment of viral packaging efficiency prior to purification and/or full capsid enrichment (e.g., cesium chloride-based density gradient, iodixanol-based density gradient or ion exchange chromatography). In some embodiments, characterization of viral vectors comprises assessing whether viral packaging efficiency is greater than or equal to 20% prior to purification and/or filtration (e.g., 20%, 25%, 30%, 35%, 40%, 45%, 50%, 55%, 60%, 65%, 70%, 75%, 80%, 85%, 90%, 95%, 99%, 100%). In some embodiments, characterization of viral vectors comprises assessment of viral packaging efficiency after purification and/or full capsid enrichment. In some embodiments, characterization of viral vectors comprises assessing whether viral packaging efficiency is greater than or equal to 50% after purification and/or filtration (e.g., 50%, 55%, 60%, 65%, 70%, 75%, 80%, 85%, 90%, 95%, 99%, 100%).

In some embodiments, methods and compositions provided herein can provide comparable or increased packaging efficiency as compared to previous methods known in the art. For example, in some embodiments, provided methods for producing and/or manufacturing viral vectors comprising use of a two-plasmid transfection system provide comparable or increased packaging efficiency as compared to a three-plasmid system. In some embodiments, provided methods for producing and/or manufacturing viral vectors comprising use of a two-plasmid transfection system with particular combinations of sequence elements provide comparable or increased packaging efficiency as compared to a two-plasmid system with a different combination of sequence elements. In some embodiments, provided methods for producing and/or manufacturing viral vectors comprising use of a two-plasmid transfection system with particular plasmid ratios provide comparable or increased packaging efficiency as compared to a two-plasmid system with different plasmid ratios.

### Replication Competent Vector Levels

In some embodiments, characterization of viral vectors comprises assessment of levels of replication competent vectors. In some embodiments, characterization of viral vectors comprises assessment of levels of replication competent vectors prior to purification and/or filtration. In some embodiments, characterization of viral vectors comprises assessment of levels of replication competent vectors after purification and/or filtration. In some embodiments, characterization of viral vectors comprises assessing whether replication competent vector levels are less than or equal to 1 rcAAV in 1E10 vg.

In some embodiments, methods and compositions provided herein can provide comparable or reduced replication competent vector levels as compared to previous methods known in the art. For example, in some embodiments, provided methods for producing viral vectors comprising use of a two-plasmid transfection system provide comparable or reduced replication competent vector levels as compared to a three-plasmid system. In some embodiments, provided methods for producing viral vectors comprising use of a two-plasmid transfection system with particular combinations of sequence elements provide comparable or reduced replication competent vector levels as compared to a two-plasmid system with a different combination of sequence elements. In some embodiments, provided methods for producing viral vectors comprise use of a two-plasmid transfection system with one or more intronic sequences inserted in the rep gene provide comparable or reduced replication competent vector levels as compared to a two-plasmid system without said intronic sequence(s).

### Wilson's Disease

Wilson's Disease (WD; OMIM 277900) is caused by variants in the ATP7B gene, encoding the copper-transporting P-type ATPase 2 protein responsible for biliary excretion of copper. As a result, deficiency in ATP7B, mainly expressed in hepatocytes, leads to decreased hepatocellular excretion of copper into bile causing abnormal deposits of copper in various tissue (Czlonkowska et al., Nat Rev Dis Primers, 2018).

WD is an autosomal recessive disorder with various symptoms related to the metabolism of copper. Symptoms vary widely and present most commonly between youth and adulthood (ages 5 and 35 years). In 1984, it was estimated that WD affected ˜1 in 30,000 individuals (Scheinber et al., Ann Neurol, 1984), however, recently a study from the United Kingdom showed, conservatively, the calculated frequency of individuals predicted to carry two mutant pathogenic ATP7B alleles is closer to ˜1 in 7000 (Coffey et al., Brain, 2013). The possible underestimations of WD prevalence may be related to the varied clinical presentation of WD and a lack of clinical diagnostic gold standards. WD clinical manifestation is multi-systemic, in which patients can experience liver, neurological/psychiatric, ophthalmologic, hematologic, renal, musculoskeletal, and/or cardiac dysfunction due to excess tissue copper accumulation (Czlonkowska et al., Nat Rev Dis Primers, 2018).

Ceruloplasmin is the main copper-binding protein in blood. It has multiple functions, including copper-dependent catalytic activities and being a source of copper ions for uptake by cells. Blood ceruloplasmin is synthesized in the liver and excreted into the circulation from hepatocytes. ATP7B, in hepatocytes, incorporates 6 copper molecules into apoceruloplasmin (not joined to copper) for the synthesis of functional ceruloplasmin and is also required for biliary copper excretion (Linder, Biomedicines, 2021). In the absence of functional ATP7B, there is reduced biliary copper excretion, reduced incorporation of copper into ceruloplasmin, and gradual copper accumulation in liver (Czlonkowska et al., Nat Rev Dis Primers, 2018). Thus, excessive quantities of non-ceruloplasmin-bound copper enters systemic circulation and copper can then accumulate in the cornea, brain, red blood cells, skeletal and cardiac muscle cells, synovial membranes, and renal cells (Roberts et al., Hepatology, 2008; Czlonkowska et al., Nat Rev Dis Primers, 2018; Leung et al., Clin Liver Dis, 2021; Sinchez-Monteagudo et al., Biomedicines, 2021).

The liver has the highest expression of ATP7B (Linder, Biomedicines, 2021) and hepatic copper accumulation causes liver injury, the earliest and most frequent manifestation of WD. Chronic hepatocyte injury and cell death leads to the progression and the development of hepatomegaly, hepatitis, cirrhosis, and liver failure. Interestingly, a study showed that the hepatic form of WD occurs more frequently in women, and women develop the neuropsychiatric form of disease later than men (Litwin et al., J Neurol Sci, 2011). Neurological and psychiatric symptoms are also frequently associated with WD, in which the clinical spectrum includes different movement disorders with a wide spectrum of involuntary movements (e.g., tremor, dystonia, parkinsonism, dysarthria, gait and posture disturbances, drooling, and dysphagia). Additionally, patient with WD may experience ophthalmological disorders including Kayser-Fleischer ring and sunflower cataract, which are caused by pathological copper accumulation in the eyes (Roberts et al., Hepatology, 2008; Czlonkowska et al., Nat Rev Dis Primers, 2018; Leung et al., Clin Liver Dis, 2021; Sinchez-Monteagudo et al., Biomedicines, 2021). Successful treatment of WD symptoms is dependent on early diagnosis to protect from disease progression.

Clinical presentation varies widely in patients with WD, thus a combination of clinical features and various tests are need to diagnosis WD. Traditionally, non-invasive laboratory tests measuring serum ceruloplasmin, urinary copper excretion, and blood levels of aspartate aminotransferase (AST) or alanine aminotransferase (ALT) can be used to establish the diagnosis. If a diagnosis cannot be established, a liver biopsy with measurement of hepatic parenchymal copper concentration may be required (Roberts et al., Hepatology, 2008; Czlonkowska et al., Nat Rev Dis Primers, 2018; Leung et al., Clin Liver Dis, 2021; Sinchez-Monteagudo et al., Biomedicines, 2021). Unfortunately, none of the available laboratory tests are fully conclusive and specific to WD. Thus, a diagnostic scoring system developed at the 8th International Meeting on Wilson disease can also be used to achieve a conclusive diagnosis (Ferenci et. al., Liver Int., 2003). Genetic analysis can provide a conclusive diagnosis.

Once a diagnosis is established, the current treatment and management options for WD involves lifelong adherence to pharmacology therapies and in the most severe cases liver transplantation. The pharmacological treatment of WD utilizes chelating agents (d-penicillamine (DPA) and trientine) that increase urinary copper excretion (Roberts et al., Hepatology, 2008; Czlonkowska et al., Nat Rev Dis Primers, 2018; Leung et al., Clin Liver Dis, 2021; Sinchez-Monteagudo et al., Biomedicines, 2021). Most patients treated with DPA therapy have hepatic improvement, however, there is a lower efficacy of DPA in neurologic WD (Brewer et al., Arch Neurol, 1987). Although DPA is the first-line treatment of WD, there are also concerns that patients can experience severe and irreversible neurological worsening at the start of treatment. Trientine is indicated for treatment of patients with Wilson's disease who are intolerant to DPA (Scheinberg et al., N Engl J Med, 1987). After initiation with chelating agents, zinc salts are administered to decrease copper absorption from the digestive tract. Ultimately, stable patients may be continued on a lower dosage of the chelating agent (as noted above) or maintained with zinc treatment (Roberts et al., Hepatology, 2008; Czlonkowska et al., Nat Rev Dis Primers, 2018; Leung et al., Clin Liver Dis, 2021; Sinchez-Monteagudo et al., Biomedicines, 2021).

As with most chronic diseases, patients and healthcare providers must consider the risks associated with nonadherence and challenges to life-long treatment. Lack of adherence with prescribed medication is a known issue for patients with WD (Maselbas et al. Neurol Neurochir Pol, 2010; Dziezyc et al. Eur J Neurol, 2014). Importantly, periods of poor adherence to pharmacological treatment can directly or indirectly influence patient outcomes as elevate levels of tissue copper presents risks for hepatic deterioration. Thus, for patients with WD, life-long adherence to treatment is critical for successful treatment.

Introduction of a functional replacement of the ATP7B gene into the genome of patients with WD would represent a much better approach, potentially providing lifelong therapeutic benefit from a single administration.

In some embodiments, a subject of the present disclosure is a neonate, infant, child, or adult. In some embodiments, a subject of the present disclosure is one week old, two weeks old, three weeks old, four weeks old, five weeks old, six weeks old, seven weeks old, eight weeks, nine weeks, ten weeks, or 12 weeks old. In some embodiments, a subject of the present disclosure is between one to three; two to four; three to five; four to six; five to seven; six to eight; six to nine; eight to ten; nine to eleven; or ten to twelve weeks old. In some embodiments, a subject of the present disclosure is less than one month old.

In some embodiments, a subject of the present disclosure is one month; two months; three months; four months; five months; six months old; seven months old; eight months old; nine months old; ten months old; eleven months old; twelve months old. In some embodiments, a subject of the present disclosure is between one to three; two to four; three to five; or four to six months old. In some embodiments, a subject of the present disclosure is between six months and 2 years old. In some embodiments, a subject of the present disclosure is between 1 and 5; 3 and 7; 5 and 9; 7 and 11; 9 and 13; 11 and 15; 13 and 17; 15 and 19; 17 and 21; 19 and 23; 21 and 25; 23 and 27; 25 and 29; 27 and 31; 29 and 33; 31 and 35 years old. In some embodiments, a subject of the present disclosure is between 30 and 40; 40 and 50; 50 and 60; 60 and 70; 70 and 80; or 80 and 90 years old.

In some embodiments, a subject has received or is receiving treatment for Wilson's Disease. In some embodiments, a method of treatment for Wilson's Disease comprises standard of care treatment. In some embodiments, a treatment for Wilson's Disease comprises DPA and/or trientine (e.g., Syprine®).

In some embodiments, methods of the present disclosure comprise administering a composition comprising a polynucleotide cassette to a subject that has received or is receiving treatment for Wilson's Disease. In some embodiments, methods of the present disclosure comprise administering a composition comprising a polynucleotide cassette to a subject that has received or is receiving DPA and/or trientine. In some embodiments, a composition comprising a polynucleotide cassette and a treatment for Wilson's Disease (e.g., DPA and/or trientine) are administered to a subject simultaneously or sequentially.

In some embodiments, administration of a composition of the present disclosure can result in modification of standard of care or prior or concurrent treatment. In some embodiments, a subject receives a lower or reduced dose of the treatment a subject was receiving prior to administration of the composition. In some embodiments, a subject stops or no longer receives the treatment a subject received prior to administration of the composition.

In some embodiments, a transgene of the present disclosure comprises a sequence encoding ATP7B or a variant thereof. In some embodiments, a transgene of the present disclosure comprises a sequence encoding a functional replacement of ATP7B. In some embodiments, a transgene of the present disclosure comprises a sequence encoding a truncated form of ATP7B. In some embodiments, a transgene of the present disclosure comprises a sequence encoding a truncated form of ATP7B as described in Huster et al., J.B.C. Vol. 278, No. 34 pp 32212-32218 the contents of which is incorporated herein in its entirety. In some embodiments, a sequence encoding ATP7B; a truncated form thereof, or a variant thereof has 80%, 85%, 90%, 95%, 99%, sequence identity to SEQ. ID NO: 14 or SEQ ID NO: 15. In some embodiments, a truncated form of ATP7B has 80%, 85%, 90%, 95%, 99%, sequence identity to SEQ. ID NO: 14 or SEQ ID NO: 15. In some embodiments, a transgene of the present disclosure is a codon-optimized variant of a sequence encoding ATP7B.

(SEQ ID NO. 33)
MPEQERQITAREGASRKILSKLSLPTRAWEPAMKKSFAFDNVGYEGGLDGLGPSSQ
PQKCFLQIKGMTCASCVSNIERNLQKEAGVLSVLVALMAGKAEIKYDPEVIQPLEIA
QFIQDLGFEAAVMEDYAGSDGNIELTITGMTCASCVHNIESKLTRINGITYASVALA
TSKALVKFDPEIIGPRDIIKIIEEIGFHASLAQRNPNAHHLDHKMEIKQWKKSFLCSLV
FGIPVMALMIYMLIPSNEPHQSMVLDHNIIPGLSILNLIFFILCTFVQLLGGWYFYVQA
YKSLRHRSANMDVLIVLATSIAYVYSLVILVVAVAEKAERSPVTFFDTPPMLFVFIAL
GRWLEHLAKSKTSEALAKLMSLQATEATVVTLGEDNLIIREEQVPMELVQRGDIVK
VVPGGKFPVDGKVLEGNTMADESLITGEAMPVTKKPGSTVIAGSINAHGSVLIKAT
HVGNDTTLAQIVKLVEEAQMSKAPIQQLADRFSGYFVPFIIIMSTLTLVVWIVIGFIDF
GVVQRYFPNPNKHISQTEVIIRFAFQTSITVLCIACPCSLGLATPTAVMVGTGVAAQN
GILIKGGKPLEMAHKIKTVMFDKTGTITHGVPRVMRVLLLGDVATLPLRKVLAVVG
TAEASSEHPLGVAVTKYCKEELGTETLGYCTDFQAVPGCGIGCKVSNVEGILAHSER
PLSAPASHLNEAGSLPAEKDAVPQTFSVLIGNREWLRRNGLTISSDVSDAMTDHEM
KGQTAILVAIDGVLCGMIAIADAVKQEAALAVHTLQSMGVDVVLITGDNRKTARAI
ATQVGINKVFAEVLPSHKVAKVQELQNKGKKVAMVGDGVNDSPALAQADMGVAI
GTGTDVAIEAADVVLIRNDLLDVVASIHLSKRTVRRIRINLVLALIYNLVGIPIAAGV
FMPIGIVLQPWMGSAAMAASSVSVVLSSLQLKCYKKPDLERYEAQAHGHMKPLTA
SQVSVHIGMDDRWRDSPRATPWDQVSYVSQVSLSSLTSDKPSRHSAAADDDGDKW
SLLLNGRDEEQYI

Because GENERIDE™ is designed to deliver therapeutic durability, it may provide lifelong benefit to patients with Wilson's Disease by intervening early in their lives with a treatment that restores the function of aberrant genes before declines in function can occur. In some embodiments, therapeutic transgenes are delivered using a GENERIDE™ construct designed to integrate immediately behind the gene coding for albumin, the most highly expressed gene in the liver. In some embodiments, expression of the transgene “piggybacks” on the expression of albumin, which may provide sufficient therapeutic levels of desirable proteins given the high level of albumin expression in the liver.

In some embodiments, compositions of the present disclosure comprise a viral vector capsid and a polynucleotide cassette as described herein. In some embodiments, a composition of the present disclosure may have 85%, 90%, 95%, 90%, 95%, 99% or 10000 sequence identity to a sequence provided below in Table 2:

TABLE 2

| | | | | | SEQ ID NO. |
|---|---|---|---|---|---|
| | | | | | (sequence |
| | | Homology | | | comprising |
| Vector | | Arm | Integration | | homology arms, |
| Name | Transgene | Lengths | site | Capsid | transgene, and P2A) |
| Vt230 | htATP7B | 0.4 kb / 0.8 kb | Human albumin | AAV-sL65 | 34 |
| Vt234 | htATP7B | 0.6 kb / 0.6 kb | Human albumin | AAV-sL65 | 35 |
| Vt229 | htATP7B | 0.8 kb / 0.4 kb | Human albumin | AAV-sL65 | 36 |
| Vt231 | htATP7B | 0.4 kb / 0.8 kb | Human albumin | AAV-LK03 | 34 |
| Vt235 | htATP7B | 0.6 kb / 0.6 kb | Human albumin | AAV-LK03 | 35 |
| Vt232 | htATP7B | 0.8 kb / 0.4 kb | Human albumin | AAV-LK03 | 36 |
| Vt212 | htATP7B | 0.6 kb / 0.6 kb | Mouse albumin | AAV-DJ | 37 |
| Vt203 | htATP7B | 1 kb / 0.6 kb | Mouse albumin | AAV-DJ | 38 |
| Vt251 | htATP7B | 0.6 kb / 0.6 kb | Cynomolgus albumin | AAV-sL65 | 39 |
| Vt213 | mtATP7B | 0.6 kb / 0.6 kb | Mouse albumin | AAV-DJ | 40 |

SEQ ID NO: 34:
GTGATGCTTATGAATATTAATAGGAATATTTGTAAGGCCTGAAATATTTTGATCA
TGAAATCAAAACATTAATTTATTTAAACATTTACTTGAAATGTGGTGGTTTGTGA
TTTAGTTGATTTTATAGGCTAGTGGGAGAATTTACATTCAAATGTCTAAATCACT
TAAAATTGCCCTTTATGGCCTGACAGTAACTTTTTTTTATTCATTTGGGGACAAC
TATGTCCGTGAGCTTCCGTCCAGAGATTATAGTAGTAAATTGTAATTAAAGGAT
ATGATGCACGTGAAATCACTTTGCAATCATCAATAGCTTCATAAATGTTAATTTT
GTATCCTAATAGTAATGCTAATATTTTCCTAACATCTGTCATGTCTTTGTGTTCA
GGGTAAAAAACTTGTTGCTGCAAGTCAAGCTGCCTTAGGCTTAGGCAGCGGCGC
CACCAACTTCAGCCTGCTGAAACAGGCCGGCGACGTGGAAGAGAACCCTGGCC
CTCCTGAGCAGGAGAGACAGATCACAGCCAGAGAAGGGGCCAGTCGGAAAATC
TTATCTAAGCTTTCTTTGCCTACCCGTGCCTGGGAACCAGCAATGAAGAAGAGT
TTTGCTTTTGACAATGTTGGCTATGAAGGTGGTCTGGATGGCCTGGGCCCTTCTT
CTCAGCCGCAGAAGTGCTTCTTACAGATCAAAGGCATGACCTGTGCATCCTGTG
TGTCTAACATAGAAAGGAATCTGCAGAAAGAAGCTGGTGTTCTCTCCGTGTTGG
TTGCCTTGATGGCAGGAAAGGCAGAGATCAAGTATGACCCAGAGGTCATCCAG
CCCCTCGAGATAGCTCAGTTCATCCAGGACCTGGGTTTTGAGGCAGCAGTCATG
GAGGACTACGCAGGCTCCGATGGCAACATTGAGCTGACAATCACAGGGATGAC
CTGCGCGTCCTGTGTCCACAACATAGAGTCCAAACTCACGAGGACAAATGGCAT
CACTTATGCCTCCGTTGCCCTTGCCACCAGCAAAGCCCTTGTTAAGTTTGACCCG
GAAATTATCGGTCCACGGGATATTATCAAAATTATTGAGGAAATTGGCTTTCAT
GCTTCCCTGGCCCAGAGAAACCCCAACGCTCATCACTTGGACCACAAGATGGAA
ATAAAGCAGTGGAAGAAGTCTTTCCTGTGCAGCCTGGTGTTTGGCATCCCTGTC
ATGGCCTTAATGATCTATATGCTGATACCCAGCAACGAGCCCCACCAGTCCATG
GTCCTGGACCACAACATCATTCCAGGACTGTCCATTCTAAATCTCATCTTCTTTA
TCTTGTGTACCTTTGTCCAGCTCCTCGGTGGGTGGTACTTCTACGTTCAGGCCTA
CAAATCTCTGAGACACAGGTCAGCCAACATGGACGTGCTCATCGTCCTGGCCAC
AAGCATTGCTTATGTTTATTCTCTGGTCATCCTGGTGGTTGCTGTGGCTGAGAAG
GCGGAGAGGAGCCCTGTGACATTCTTCGACACGCCCCCCATGCTCTTTGTGTTCA
TTGCCCTGGGCCGGTGGCTGGAACACTTGGCAAAGAGCAAAACCTCAGAAGCC
CTGGCTAAACTCATGTCTCTCCAAGCCACAGAAGCCACCGTTGTGACCCTTGGT
GAGGACAATTTAATCATCAGGGAGGAGCAAGTCCCCATGGAGCTGGTGCAGCG
GGGCGATATCGTCAAGGTGGTCCCTGGGGGAAAGTTTCCAGTGGATGGGAAAG
TCCTGGAAGGCAATACCATGGCTGATGAGTCCCTCATCACAGGAGAAGCCATGC
CAGTCACTAAGAAACCCGGAAGCACTGTAATTGCGGGGTCTATAAATGCACATG
GCTCTGTGCTCATTAAAGCTACCCACGTGGGCAATGACACCACTTTGGCTCAGA
TTGTGAAACTGGTGGAAGAGGCTCAGATGTCAAAGGCACCCATTCAGCAGCTGG
CTGACCGGTTTAGTGGATATTTTGTCCCATTTATCATCATCATGTCAACTTTGAC
GTTGGTGGTATGGATTGTAATCGGTTTTATCGATTTTGGTGTTGTTCAGAGATAC
TTTCCTAACCCCAACAAGCACATCTCCCAGACAGAGGTGATCATCCGGTTTGCTT
TCCAGACGTCCATCACGGTGCTGTGCATTGCCTGCCCCTGCTCCCTGGGGCTGGC
CACGCCCACGGCTGTCATGGTGGGCACCGGGGTGGCCGCGCAGAACGGCATCCT
CATCAAGGGAGGCAAGCCCCTGGAGATGGCGCACAAGATAAAGACTGTGATGT
TTGACAAGACTGGCACCATTACCCATGGCGTCCCCAGGGTCATGCGGGTGCTCC
TGCTGGGGGATGTGGCCACACTGCCCCTCAGGAAGGTTCTGGCTGTGGTGGGGA
CTGCGGAGGCCAGCAGTGAACACCCCTTGGGCGTGGCAGTCACCAAATACTGTA
AAGAGGAACTTGGAACAGAGACCTTGGGATACTGCACGGACTTCCAGGCAGTG
CCAGGCTGTGGAATTGGGTGCAAAGTCAGCAACGTGGAAGGCATCCTGGCCCA
CAGTGAGCGCCCTTTGAGTGCACCGGCCAGTCACCTGAATGAGGCTGGCAGCCT
TCCCGCAGAAAAAGATGCAGTCCCCCAGACCTTCTCTGTGCTGATTGGAAACCG
TGAGTGGCTGAGGCGCAACGGTTTAACCATTTCTAGCGATGTCAGTGACGCTAT
GACAGACCACGAGATGAAAGGACAGACAGCCATCCTGGTGGCTATTGACGGTG
TGCTCTGTGGGATGATCGCAATCGCAGACGCTGTCAAGCAGGAGGCTGCCCTGG
CTGTGCACACGCTGCAGAGCATGGGTGTGGACGTGGTTCTGATCACGGGGGACA
ACCGGAAGACAGCCAGAGCTATTGCCACCCAGGTTGGCATCAACAAAGTCTTTG
CAGAGGTGCTGCCTTCGCACAAGGTGGCCAAGGTCCAGGAGCTCCAGAATAAA
GGGAAGAAAGTCGCCATGGTGGGGGATGGGGTCAATGACTCCCCGGCCTTGGC
CCAGGCAGACATGGGTGTGGCCATTGGCACCGGCACGGATGTGGCCATCGAGG
CAGCCGACGTCGTCCTTATCAGAAATGATTTGCTGGATGTGGTGGCTAGCATTC
ACCTTTCCAAGAGGACTGTCCGAAGGATACGCATCAACCTGGTCCTGGCACTGA
TTTATAACCTGGTTGGGATACCCATTGCAGCAGGTGTCTTCATGCCCATCGGCAT
TGTGCTGCAGCCCTGGATGGGCTCAGCGGCCATGGCAGCCTCCTCTGTGTCTGT
GGTGCTCTCATCCCTGCAGCTCAAGTGCTATAAGAAGCCTGACCTGGAGAGGTA
TGAGGCACAGGCGCATGGCCACATGAAGCCCCTGACGGCATCCCAGGTCAGTGT
GCACATAGGCATGGATGACAGGTGGCGGGACTCCCCCAGGGCCACACCATGGG
ACCAGGTCAGCTATGTCAGCCAGGTGTCGCTGTCCTCCCTGACGTCCGACAAGC
CATCTCGGCACAGCGCTGCAGCAGACGATGATGGGGACAAGTGGTCTCTGCTCC
TGAATGGCAGGGATGAGGAGCAGTACATCTAACATCACATTTAAAAGCATCTCA
GGTAACTATATTTTGAATTTTTTAAAAAAGTAACTATAATAGTTATTATTAAAAT
AGCAAAGATTGACCATTTCCAAGAGCCATATAGACCAGCACCGACCACTATTCT
AAACTATTTATGTATGTAAATATTAGCTTTTAAAATTCTCAAAATAGTTGCTGAG
TTGGGAACCACTATTATTTCTATTTTGTAGATGAGAAAATGAAGATAAACATCA
AAGCATAGATTAAGTAATTTTCCAAAGGGTCAAAATTCAAAATTGAAACCAAAG
TTTCAGTGTTGCCCATTGTCCTGTTCTGACTTATATGATGCGGTACACAGAGCCA
TCCAAGTAAGTGATGGCTCAGCAGTGGAATACTCTGGGAATTAGGCTGAACCAC
ATGAAAGAGTGCTTTATAGGGCAAAAACAGTTGAATATCAGTGATTTCACATGG
TTCAACCTAATAGTTCAACTCATCCTTTCCATTGGAGAATATGATGGATCTACCT
TCTGTGAACTTTATAGTGAAGAATCTGCTATTACATTTCCAATTTGTCAACATGC
TGAGCTTTAATAGGACTTATCTTCTTATGACAACATTTATTGGTGTGTCCCCTTG
CCTAGCCCAACAGAAGAATTCAGCAGCCGTAAGTCTAGGACAGGCTTAAATTGT
TTTCACTGGTGTAAATTGCAGAAAGATGATCTAAGTAATTTGGCATTTATTTTAA
TAGGTTTGAAAAACACATGCCATTTTACAAATAAGACTTATATTTGTCCTTTTGT
TTTTCAGCCTACCATGAG
SEQ ID NO: 35:
GCATGTTTGGTTAGGCTAGGGCTTAGGGATTTATATATCAAAGGAGGCTTTGTA
CATGTGGGACAGGGATCTTATTTTACAAACAATTGTCTTACAAAATGAATAAAA
CAGCACTTTGTTTTTATCTCCTGCTCTATTGTGCCATACTGTTAAATGTTTATAAT
GCCTGTTCTGTTTCCAAATTTGTGATGCTTATGAATATTAATAGGAATATTTGTA
AGGCCTGAAATATTTTGATCATGAAATCAAAACATTAATTTATTTAAACATTTAC
TTGAAATGTGGTGGTTTGTGATTTAGTTGATTTTATAGGCTAGTGGGAGAATTTA
CATTCAAATGTCTAAATCACTTAAAATTGCCCTTTATGGCCTGACAGTAACTTTT
TTTTATTCATTTGGGGACAACTATGTCCGTGAGCTTCCGTCCAGAGATTATAGTA
GTAAATTGTAATTAAAGGATATGATGCACGTGAAATCACTTTGCAATCATCAAT
AGCTTCATAAATGTTAATTTTGTATCCTAATAGTAATGCTAATATTTTCCTAACA
TCTGTCATGTCTTTGTGTTCAGGGTAAAAAACTTGTTGCTGCAAGTCAAGCTGCC
TTAGGCTTAGGCAGCGGCGCCACCAACTTCAGCCTGCTGAAACAGGCCGGCGAC
GTGGAAGAGAACCCTGGCCCTCCTGAGCAGGAGAGACAGATCACAGCCAGAGA
AGGGGCCAGTCGGAAAATCTTATCTAAGCTTTCTTTGCCTACCCGTGCCTGGGA
ACCAGCAATGAAGAAGAGTTTTGCTTTTGACAATGTTGGCTATGAAGGTGGTCT
GGATGGCCTGGGCCCTTCTTCTCAGCCGCAGAAGTGCTTCTTACAGATCAAAGG
CATGACCTGTGCATCCTGTGTGTCTAACATAGAAAGGAATCTGCAGAAAGAAGC
TGGTGTTCTCTCCGTGTTGGTTGCCTTGATGGCAGGAAAGGCAGAGATCAAGTA
TGACCCAGAGGTCATCCAGCCCCTCGAGATAGCTCAGTTCATCCAGGACCTGGG
TTTTGAGGCAGCAGTCATGGAGGACTACGCAGGCTCCGATGGCAACATTGAGCT
GACAATCACAGGGATGACCTGCGCGTCCTGTGTCCACAACATAGAGTCCAAACT
CACGAGGACAAATGGCATCACTTATGCCTCCGTTGCCCTTGCCACCAGCAAAGC
CCTTGTTAAGTTTGACCCGGAAATTATCGGTCCACGGGATATTATCAAAATTATT
GAGGAAATTGGCTTTCATGCTTCCCTGGCCCAGAGAAACCCCAACGCTCATCAC
TTGGACCACAAGATGGAAATAAAGCAGTGGAAGAAGTCTTTCCTGTGCAGCCTG
GTGTTTGGCATCCCTGTCATGGCCTTAATGATCTATATGCTGATACCCAGCAACG
AGCCCCACCAGTCCATGGTCCTGGACCACAACATCATTCCAGGACTGTCCATTC
TAAATCTCATCTTCTTTATCTTGTGTACCTTTGTCCAGCTCCTCGGTGGGTGGTAC
TTCTACGTTCAGGCCTACAAATCTCTGAGACACAGGTCAGCCAACATGGACGTG
CTCATCGTCCTGGCCACAAGCATTGCTTATGTTTATTCTCTGGTCATCCTGGTGG
TTGCTGTGGCTGAGAAGGCGGAGAGGAGCCCTGTGACATTCTTCGACACGCCCC
CCATGCTCTTTGTGTTCATTGCCCTGGGCCGGTGGCTGGAACACTTGGCAAAGA
GCAAAACCTCAGAAGCCCTGGCTAAACTCATGTCTCTCCAAGCCACAGAAGCCA
CCGTTGTGACCCTTGGTGAGGACAATTTAATCATCAGGGAGGAGCAAGTCCCCA
TGGAGCTGGTGCAGCGGGGCGATATCGTCAAGGTGGTCCCTGGGGGAAAGTTTC
CAGTGGATGGGAAAGTCCTGGAAGGCAATACCATGGCTGATGAGTCCCTCATCA
CAGGAGAAGCCATGCCAGTCACTAAGAAACCCGGAAGCACTGTAATTGCGGGG
TCTATAAATGCACATGGCTCTGTGCTCATTAAAGCTACCCACGTGGGCAATGAC
ACCACTTTGGCTCAGATTGTGAAACTGGTGGAAGAGGCTCAGATGTCAAAGGCA
CCCATTCAGCAGCTGGCTGACCGGTTTAGTGGATATTTTGTCCCATTTATCATCA
TCATGTCAACTTTGACGTTGGTGGTATGGATTGTAATCGGTTTTATCGATTTTGG
TGTTGTTCAGAGATACTTTCCTAACCCCAACAAGCACATCTCCCAGACAGAGGT
GATCATCCGGTTTGCTTTCCAGACGTCCATCACGGTGCTGTGCATTGCCTGCCCC
TGCTCCCTGGGGCTGGCCACGCCCACGGCTGTCATGGTGGGCACCGGGGTGGCC
GCGCAGAACGGCATCCTCATCAAGGGAGGCAAGCCCCTGGAGATGGCGCACAA
GATAAAGACTGTGATGTTTGACAAGACTGGCACCATTACCCATGGCGTCCCCAG
GGTCATGCGGGTGCTCCTGCTGGGGGATGTGGCCACACTGCCCCTCAGGAAGGT
TCTGGCTGTGGTGGGGACTGCGGAGGCCAGCAGTGAACACCCCTTGGGCGTGGC
AGTCACCAAATACTGTAAAGAGGAACTTGGAACAGAGACCTTGGGATACTGCA
CGGACTTCCAGGCAGTGCCAGGCTGTGGAATTGGGTGCAAAGTCAGCAACGTG
GAAGGCATCCTGGCCCACAGTGAGCGCCCTTTGAGTGCACCGGCCAGTCACCTG
AATGAGGCTGGCAGCCTTCCCGCAGAAAAAGATGCAGTCCCCCAGACCTTCTCT
GTGCTGATTGGAAACCGTGAGTGGCTGAGGCGCAACGGTTTAACCATTTCTAGC
GATGTCAGTGACGCTATGACAGACCACGAGATGAAAGGACAGACAGCCATCCT
GGTGGCTATTGACGGTGTGCTCTGTGGGATGATCGCAATCGCAGACGCTGTCAA
GCAGGAGGCTGCCCTGGCTGTGCACACGCTGCAGAGCATGGGTGTGGACGTGGT
TCTGATCACGGGGGACAACCGGAAGACAGCCAGAGCTATTGCCACCCAGGTTG
GCATCAACAAAGTCTTTGCAGAGGTGCTGCCTTCGCACAAGGTGGCCAAGGTCC
AGGAGCTCCAGAATAAAGGGAAGAAAGTCGCCATGGTGGGGGATGGGGTCAAT
GACTCCCCGGCCTTGGCCCAGGCAGACATGGGTGTGGCCATTGGCACCGGCACG
GATGTGGCCATCGAGGCAGCCGACGTCGTCCTTATCAGAAATGATTTGCTGGAT
GTGGTGGCTAGCATTCACCTTTCCAAGAGGACTGTCCGAAGGATACGCATCAAC
CTGGTCCTGGCACTGATTTATAACCTGGTTGGGATACCCATTGCAGCAGGTGTCT
TCATGCCCATCGGCATTGTGCTGCAGCCCTGGATGGGCTCAGCGGCCATGGCAG
CCTCCTCTGTGTCTGTGGTGCTCTCATCCCTGCAGCTCAAGTGCTATAAGAAGCC
TGACCTGGAGAGGTATGAGGCACAGGCGCATGGCCACATGAAGCCCCTGACGG
CATCCCAGGTCAGTGTGCACATAGGCATGGATGACAGGTGGCGGGACTCCCCCA
GGGCCACACCATGGGACCAGGTCAGCTATGTCAGCCAGGTGTCGCTGTCCTCCC
TGACGTCCGACAAGCCATCTCGGCACAGCGCTGCAGCAGACGATGATGGGGAC
AAGTGGTCTCTGCTCCTGAATGGCAGGGATGAGGAGCAGTACATCTAACATCAC
ATTTAAAAGCATCTCAGGTAACTATATTTTGAATTTTTTAAAAAAGTAACTATAA
TAGTTATTATTAAAATAGCAAAGATTGACCATTTCCAAGAGCCATATAGACCAG
CACCGACCACTATTCTAAACTATTTATGTATGTAAATATTAGCTTTTAAAATTCT
CAAAATAGTTGCTGAGTTGGGAACCACTATTATTTCTATTTTGTAGATGAGAAA
ATGAAGATAAACATCAAAGCATAGATTAAGTAATTTTCCAAAGGGTCAAAATTC
AAAATTGAAACCAAAGTTTCAGTGTTGCCCATTGTCCTGTTCTGACTTATATGAT
GCGGTACACAGAGCCATCCAAGTAAGTGATGGCTCAGCAGTGGAATACTCTGG
GAATTAGGCTGAACCACATGAAAGAGTGCTTTATAGGGCAAAAACAGTTGAAT
ATCAGTGATTTCACATGGTTCAACCTAATAGTTCAACTCATCCTTTCCATTGGAG
AATATGATGGATCTACCTTCTGTGAACTTTATAGTGAAGAATCTGCTATTACATT
TCCAATTTGTCAACATGCTGAGCTTTAATAGGACTTATCTTCTTATGACAACATT
TATTG
SEQ ID NO: 36:
TTCAAACTCAGTGCACTTGTTGAGCTTGTGAAACACAAGCCCAAGGCAACAAAA
GAGCAACTGAAAGCTGTTATGGATGATTTCGCAGCTTTTGTAGAGAAGTGCTGC
AAGGCTGACGATAAGGAGACCTGCTTTGCCGAGGAGGTACTACAGTTCTCTTCA
TTTTAATATGTCCAGTATTCATTTTTGCATGTTTGGTTAGGCTAGGGCTTAGGGA
TTTATATATCAAAGGAGGCTTTGTACATGTGGGACAGGGATCTTATTTTACAAA
CAATTGTCTTACAAAATGAATAAAACAGCACTTTGTTTTTATCTCCTGCTCTATT
GTGCCATACTGTTAAATGTTTATAATGCCTGTTCTGTTTCCAAATTTGTGATGCTT
ATGAATATTAATAGGAATATTTGTAAGGCCTGAAATATTTTGATCATGAAATCA
AAACATTAATTTATTTAAACATTTACTTGAAATGTGGTGGTTTGTGATTTAGTTG
ATTTTATAGGCTAGTGGGAGAATTTACATTCAAATGTCTAAATCACTTAAAATTG
CCCTTTATGGCCTGACAGTAACTTTTTTTTATTCATTTGGGGACAACTATGTCCG
TGAGCTTCCGTCCAGAGATTATAGTAGTAAATTGTAATTAAAGGATATGATGCA
CGTGAAATCACTTTGCAATCATCAATAGCTTCATAAATGTTAATTTTGTATCCTA
ATAGTAATGCTAATATTTTCCTAACATCTGTCATGTCTTTGTGTTCAGGGTAAAA
AACTTGTTGCTGCAAGTCAAGCTGCCTTAGGCTTAGGCAGCGGCGCCACCAACT
TCAGCCTGCTGAAACAGGCCGGCGACGTGGAAGAGAACCCTGGCCCTCCTGAG
CAGGAGAGACAGATCACAGCCAGAGAAGGGGCCAGTCGGAAAATCTTATCTAA
GCTTTCTTTGCCTACCCGTGCCTGGGAACCAGCAATGAAGAAGAGTTTTGCTTTT
GACAATGTTGGCTATGAAGGTGGTCTGGATGGCCTGGGCCCTTCTTCTCAGCCG
CAGAAGTGCTTCTTACAGATCAAAGGCATGACCTGTGCATCCTGTGTGTCTAAC
ATAGAAAGGAATCTGCAGAAAGAAGCTGGTGTTCTCTCCGTGTTGGTTGCCTTG
ATGGCAGGAAAGGCAGAGATCAAGTATGACCCAGAGGTCATCCAGCCCCTCGA
GATAGCTCAGTTCATCCAGGACCTGGGTTTTGAGGCAGCAGTCATGGAGGACTA
CGCAGGCTCCGATGGCAACATTGAGCTGACAATCACAGGGATGACCTGCGCGTC
CTGTGTCCACAACATAGAGTCCAAACTCACGAGGACAAATGGCATCACTTATGC
CTCCGTTGCCCTTGCCACCAGCAAAGCCCTTGTTAAGTTTGACCCGGAAATTATC
GGTCCACGGGATATTATCAAAATTATTGAGGAAATTGGCTTTCATGCTTCCCTGG
CCCAGAGAAACCCCAACGCTCATCACTTGGACCACAAGATGGAAATAAAGCAG
TGGAAGAAGTCTTTCCTGTGCAGCCTGGTGTTTGGCATCCCTGTCATGGCCTTAA
TGATCTATATGCTGATACCCAGCAACGAGCCCCACCAGTCCATGGTCCTGGACC
ACAACATCATTCCAGGACTGTCCATTCTAAATCTCATCTTCTTTATCTTGTGTAC
CTTTGTCCAGCTCCTCGGTGGGTGGTACTTCTACGTTCAGGCCTACAAATCTCTG
AGACACAGGTCAGCCAACATGGACGTGCTCATCGTCCTGGCCACAAGCATTGCT
TATGTTTATTCTCTGGTCATCCTGGTGGTTGCTGTGGCTGAGAAGGCGGAGAGG
AGCCCTGTGACATTCTTCGACACGCCCCCCATGCTCTTTGTGTTCATTGCCCTGG
GCCGGTGGCTGGAACACTTGGCAAAGAGCAAAACCTCAGAAGCCCTGGCTAAA
CTCATGTCTCTCCAAGCCACAGAAGCCACCGTTGTGACCCTTGGTGAGGACAAT
TTAATCATCAGGGAGGAGCAAGTCCCCATGGAGCTGGTGCAGCGGGGCGATAT
CGTCAAGGTGGTCCCTGGGGGAAAGTTTCCAGTGGATGGGAAAGTCCTGGAAG
GCAATACCATGGCTGATGAGTCCCTCATCACAGGAGAAGCCATGCCAGTCACTA
AGAAACCCGGAAGCACTGTAATTGCGGGGTCTATAAATGCACATGGCTCTGTGC
TCATTAAAGCTACCCACGTGGGCAATGACACCACTTTGGCTCAGATTGTGAAAC
TGGTGGAAGAGGCTCAGATGTCAAAGGCACCCATTCAGCAGCTGGCTGACCGGT
TTAGTGGATATTTTGTCCCATTTATCATCATCATGTCAACTTTGACGTTGGTGGT
ATGGATTGTAATCGGTTTTATCGATTTTGGTGTTGTTCAGAGATACTTTCCTAAC
CCCAACAAGCACATCTCCCAGACAGAGGTGATCATCCGGTTTGCTTTCCAGACG
TCCATCACGGTGCTGTGCATTGCCTGCCCCTGCTCCCTGGGGCTGGCCACGCCCA
CGGCTGTCATGGTGGGCACCGGGGTGGCCGCGCAGAACGGCATCCTCATCAAG
GGAGGCAAGCCCCTGGAGATGGCGCACAAGATAAAGACTGTGATGTTTGACAA
GACTGGCACCATTACCCATGGCGTCCCCAGGGTCATGCGGGTGCTCCTGCTGGG
GGATGTGGCCACACTGCCCCTCAGGAAGGTTCTGGCTGTGGTGGGGACTGCGGA
GGCCAGCAGTGAACACCCCTTGGGCGTGGCAGTCACCAAATACTGTAAAGAGG
AACTTGGAACAGAGACCTTGGGATACTGCACGGACTTCCAGGCAGTGCCAGGCT
GTGGAATTGGGTGCAAAGTCAGCAACGTGGAAGGCATCCTGGCCCACAGTGAG
CGCCCTTTGAGTGCACCGGCCAGTCACCTGAATGAGGCTGGCAGCCTTCCCGCA
GAAAAAGATGCAGTCCCCCAGACCTTCTCTGTGCTGATTGGAAACCGTGAGTGG
CTGAGGCGCAACGGTTTAACCATTTCTAGCGATGTCAGTGACGCTATGACAGAC
CACGAGATGAAAGGACAGACAGCCATCCTGGTGGCTATTGACGGTGTGCTCTGT
GGGATGATCGCAATCGCAGACGCTGTCAAGCAGGAGGCTGCCCTGGCTGTGCAC
ACGCTGCAGAGCATGGGTGTGGACGTGGTTCTGATCACGGGGGACAACCGGAA
GACAGCCAGAGCTATTGCCACCCAGGTTGGCATCAACAAAGTCTTTGCAGAGGT
GCTGCCTTCGCACAAGGTGGCCAAGGTCCAGGAGCTCCAGAATAAAGGGAAGA
AAGTCGCCATGGTGGGGGATGGGGTCAATGACTCCCCGGCCTTGGCCCAGGCAG
ACATGGGTGTGGCCATTGGCACCGGCACGGATGTGGCCATCGAGGCAGCCGAC
GTCGTCCTTATCAGAAATGATTTGCTGGATGTGGTGGCTAGCATTCACCTTTCCA
AGAGGACTGTCCGAAGGATACGCATCAACCTGGTCCTGGCACTGATTTATAACC
TGGTTGGGATACCCATTGCAGCAGGTGTCTTCATGCCCATCGGCATTGTGCTGCA
GCCCTGGATGGGCTCAGCGGCCATGGCAGCCTCCTCTGTGTCTGTGGTGCTCTCA
TCCCTGCAGCTCAAGTGCTATAAGAAGCCTGACCTGGAGAGGTATGAGGCACAG
GCGCATGGCCACATGAAGCCCCTGACGGCATCCCAGGTCAGTGTGCACATAGGC
ATGGATGACAGGTGGCGGGACTCCCCCAGGGCCACACCATGGGACCAGGTCAG
CTATGTCAGCCAGGTGTCGCTGTCCTCCCTGACGTCCGACAAGCCATCTCGGCA
CAGCGCTGCAGCAGACGATGATGGGGACAAGTGGTCTCTGCTCCTGAATGGCAG
GGATGAGGAGCAGTACATCTAACATCACATTTAAAAGCATCTCAGGTAACTATA
TTTTGAATTTTTTAAAAAAGTAACTATAATAGTTATTATTAAAATAGCAAAGATT
GACCATTTCCAAGAGCCATATAGACCAGCACCGACCACTATTCTAAACTATTTA
TGTATGTAAATATTAGCTTTTAAAATTCTCAAAATAGTTGCTGAGTTGGGAACCA
CTATTATTTCTATTTTGTAGATGAGAAAATGAAGATAAACATCAAAGCATAGAT
TAAGTAATTTTCCAAAGGGTCAAAATTCAAAATTGAAACCAAAGTTTCAGTGTT
GCCCATTGTCCTGTTCTGACTTATATGATGCGGTACACAGAGCCATCCAAGTAA
GTGATGGCTCAGCAGTGGAATACTCTGGGAATTAGGCTGAACCACATGAAAGA
GTGCTTTATA
SEQ ID NO: 37:
GACTGAGGTCAGAAACGTTTTTGCATTTTGACGATGTTCAGTTTCCATTTTCTGT
GCACGTGGTCAGGTGTAGCTCTCTGGAACTCACACACTGAATAACTCCACCAAT
CTAGATGTTGTTCTCTACGTAACTGTAATAGAAACTGACTTACGTAGCTTTTAAT
TTTTATTTTCTGCCACACTGCTGCCTATTAAATACCTATTATCACTATTTGGTTTC
AAATTTGTGACACAGAAGAGCATAGTTAGAAATACTTGCAAAGCCTAGAATCAT
GAACTCATTTAAACCTTGCCCTGAAATGTTTCTTTTTGAATTGAGTTATTTTACA
CATGAATGGACAGTTACCATTATATATCTGAATCATTTCACATTCCCTCCCATGG
CCTAACAACAGTTTATCTTCTTATTTTGGGCACAACAGATGTCAGAGAGCCTGCT
TTAGGAATTCTAAGTAGAACTGTAATTAAGCAATGCAAGGCACGTACGTTTACT
ATGTCATTGCCTATGGCTATGAAGTGCAAATCCTAACAGTCCTGCTAATACTTTT
CTAACATCCATCATTTCTTTGTTTTCAGGGTCCAAACCTTGTCACTAGATGCAAA
GACGCCTTAGCCGGAAGCGGCGCCACCAATTTCAGCCTGCTGAAACAGGCCGGC
GACGTGGAAGAGAACCCTGGCCCTCCTGAGCAGGAGAGACAGATCACAGCCAG
AGAAGGGGCCAGTCGGAAAATCTTATCTAAGCTTTCTTTGCCTACCCGTGCCTG
GGAACCAGCAATGAAGAAGAGTTTTGCTTTTGACAATGTTGGCTATGAAGGTGG
TCTGGATGGCCTGGGCCCTTCTTCTCAGCCGCAGAAGTGCTTCTTACAGATCAAA
GGCATGACCTGTGCATCCTGTGTGTCTAACATAGAAAGGAATCTGCAGAAAGAA
GCTGGTGTTCTCTCCGTGTTGGTTGCCTTGATGGCAGGAAAGGCAGAGATCAAG
TATGACCCAGAGGTCATCCAGCCCCTCGAGATAGCTCAGTTCATCCAGGACCTG
GGTTTTGAGGCAGCAGTCATGGAGGACTACGCAGGCTCCGATGGCAACATTGAG
CTGACAATCACAGGGATGACCTGCGCGTCCTGTGTCCACAACATAGAGTCCAAA
CTCACGAGGACAAATGGCATCACTTATGCCTCCGTTGCCCTTGCCACCAGCAAA
GCCCTTGTTAAGTTTGACCCGGAAATTATCGGTCCACGGGATATTATCAAAATT
ATTGAGGAAATTGGCTTTCATGCTTCCCTGGCCCAGAGAAACCCCAACGCTCAT
CACTTGGACCACAAGATGGAAATAAAGCAGTGGAAGAAGTCTTTCCTGTGCAGC
CTGGTGTTTGGCATCCCTGTCATGGCCTTAATGATCTATATGCTGATACCCAGCA
ACGAGCCCCACCAGTCCATGGTCCTGGACCACAACATCATTCCAGGACTGTCCA
TTCTAAATCTCATCTTCTTTATCTTGTGTACCTTTGTCCAGCTCCTCGGTGGGTGG
TACTTCTACGTTCAGGCCTACAAATCTCTGAGACACAGGTCAGCCAACATGGAC
GTGCTCATCGTCCTGGCCACAAGCATTGCTTATGTTTATTCTCTGGTCATCCTGG
TGGTTGCTGTGGCTGAGAAGGCGGAGAGGAGCCCTGTGACATTCTTCGACACGC
CCCCCATGCTCTTTGTGTTCATTGCCCTGGGCCGGTGGCTGGAACACTTGGCAAA
GAGCAAAACCTCAGAAGCCCTGGCTAAACTCATGTCTCTCCAAGCCACAGAAGC
CACCGTTGTGACCCTTGGTGAGGACAATTTAATCATCAGGGAGGAGCAAGTCCC
CATGGAGCTGGTGCAGCGGGGCGATATCGTCAAGGTGGTCCCTGGGGGAAAGT
TTCCAGTGGATGGGAAAGTCCTGGAAGGCAATACCATGGCTGATGAGTCCCTCA
TCACAGGAGAAGCCATGCCAGTCACTAAGAAACCCGGAAGCACTGTAATTGCG
GGGTCTATAAATGCACATGGCTCTGTGCTCATTAAAGCTACCCACGTGGGCAAT
GACACCACTTTGGCTCAGATTGTGAAACTGGTGGAAGAGGCTCAGATGTCAAAG
GCACCCATTCAGCAGCTGGCTGACCGGTTTAGTGGATATTTTGTCCCATTTATCA
TCATCATGTCAACTTTGACGTTGGTGGTATGGATTGTAATCGGTTTTATCGATTT
TGGTGTTGTTCAGAGATACTTTCCTAACCCCAACAAGCACATCTCCCAGACAGA
GGTGATCATCCGGTTTGCTTTCCAGACGTCCATCACGGTGCTGTGCATTGCCTGC
CCCTGCTCCCTGGGGCTGGCCACGCCCACGGCTGTCATGGTGGGCACCGGGGTG
GCCGCGCAGAACGGCATCCTCATCAAGGGAGGCAAGCCCCTGGAGATGGCGCA
CAAGATAAAGACTGTGATGTTTGACAAGACTGGCACCATTACCCATGGCGTCCC
CAGGGTCATGCGGGTGCTCCTGCTGGGGGATGTGGCCACACTGCCCCTCAGGAA
GGTTCTGGCTGTGGTGGGGACTGCGGAGGCCAGCAGTGAACACCCCTTGGGCGT
GGCAGTCACCAAATACTGTAAAGAGGAACTTGGAACAGAGACCTTGGGATACT
GCACGGACTTCCAGGCAGTGCCAGGCTGTGGAATTGGGTGCAAAGTCAGCAAC
GTGGAAGGCATCCTGGCCCACAGTGAGCGCCCTTTGAGTGCACCGGCCAGTCAC
CTGAATGAGGCTGGCAGCCTTCCCGCAGAAAAAGATGCAGTCCCCCAGACCTTC
TCTGTGCTGATTGGAAACCGTGAGTGGCTGAGGCGCAACGGTTTAACCATTTCT
AGCGATGTCAGTGACGCTATGACAGACCACGAGATGAAAGGACAGACAGCCAT
CCTGGTGGCTATTGACGGTGTGCTCTGTGGGATGATCGCAATCGCAGACGCTGT
CAAGCAGGAGGCTGCCCTGGCTGTGCACACGCTGCAGAGCATGGGTGTGGACG
TGGTTCTGATCACGGGGGACAACCGGAAGACAGCCAGAGCTATTGCCACCCAG
GTTGGCATCAACAAAGTCTTTGCAGAGGTGCTGCCTTCGCACAAGGTGGCCAAG
GTCCAGGAGCTCCAGAATAAAGGGAAGAAAGTCGCCATGGTGGGGGATGGGGT
CAATGACTCCCCGGCCTTGGCCCAGGCAGACATGGGTGTGGCCATTGGCACCGG
CACGGATGTGGCCATCGAGGCAGCCGACGTCGTCCTTATCAGAAATGATTTGCT
GGATGTGGTGGCTAGCATTCACCTTTCCAAGAGGACTGTCCGAAGGATACGCAT
CAACCTGGTCCTGGCACTGATTTATAACCTGGTTGGGATACCCATTGCAGCAGG
TGTCTTCATGCCCATCGGCATTGTGCTGCAGCCCTGGATGGGCTCAGCGGCCAT
GGCAGCCTCCTCTGTGTCTGTGGTGCTCTCATCCCTGCAGCTCAAGTGCTATAAG
AAGCCTGACCTGGAGAGGTATGAGGCACAGGCGCATGGCCACATGAAGCCCCT
GACGGCATCCCAGGTCAGTGTGCACATAGGCATGGATGACAGGTGGCGGGACT
CCCCCAGGGCCACACCATGGGACCAGGTCAGCTATGTCAGCCAGGTGTCGCTGT
CCTCCCTGACGTCCGACAAGCCATCTCGGCACAGCGCTGCAGCAGACGATGATG
GGGACAAGTGGTCTCTGCTCCTGAATGGCAGGGATGAGGAGCAGTACATCTAA
ACACATCACAACCACAACCTTCTCAGGTAACTATACTTGGGACTTAAAAAACAT
AATCATAATCATTTTTCCTAAAACGATCAAGACTGATAACCATTTGACAAGAGC
CATACAGACAAGCACCAGCTGGCACTCTTAGGTCTTCACGTATGGTCATCAGTT
TGGGTTCCATTTGTAGATAAGAAACTGAACATATAAAGGTCTAGGTTAATGCAA
TTTACACAAAAGGAGACCAAACCAGGGAGAGAAGGAACCAAAATTAAAAATTC
AAACCAGAGCAAAGGAGTTAGCCCTGGTTTTGCTCTGACTTACATGAACCACTA
TGTGGAGTCCTCCATGTTAGCCTAGTCAAGCTTATCCTCTGGATGAAGTTGAAAC
CATATGAAGGAATATTTGGGGGGTGGGTCAAAACAGTTGTGTATCAATGATTCC
ATGTGGTTTGACCCAATCATTCTGTGAATCCATTTCAACAGAAGATACAACGGG
TTCTGTTTCATAATAAGTGATCCACTTCCAAATTTCTGATGTGCCCCATGCTAAG
CTTTAACAGAATTTATCTTCTTATGACAAAGCAGCCTCCTTTGAAAATATAGCCA
ACTGCACACAGCTATG
SEQ ID NO: 38:
GTAATGCATGGATCCCCTAGGGCGGCCGCCTGAAACTAGACAAAACCCGTGTGA
CTGGCATCGATTATTCTATTTGATCTAGCTAGTCCTAGCAAAGTGACAACTGCTA
CTCCCCTCCTACACAGCCAAGATTCCTAAGTTGGCAGTGGCATGCTTAATCCTCA
AAGCCAAAGTTACTTGGCTCCAAGATTTATAGCCTTAAACTGTGGCCTCACATTC
CTTCCTATCTTACTTTCCTGCACTGGGGTAAATGTCTCCTTGCTCTTCTTGCTTTC
TGTCCTACTGCAGGGCTCTTGCTGAGCTGGTGAAGCACAAGCCCAAGGCTACAG
CGGAGCAACTGAAGACTGTCATGGATGACTTTGCACAGTTCCTGGATACATGTT
GCAAGGCTGCTGACAAGGACACCTGCTTCTCGACTGAGGTCAGAAACGTTTTTG
CATTTTGACGATGTTCAGTTTCCATTTTCTGTGCACGTGGTCAGGTGTAGCTCTC
TGGAACTCACACACTGAATAACTCCACCAATCTAGATGTTGTTCTCTACCGAGA
CTGAGGTCAGAAACGTTTTTGCATTTTGACGATGTTCAGTTTCCATTTTCTGTGC
ACGTGGTCAGGTGTAGCTCTCTGGAACTCACACACTGAATAACTCCACCAATCT
AGATGTTGTTCTCTACGTAACTGTAATAGAAACTGACTTACGTAGCTTTTAATTT
TTATTTTCTGCCACACTGCTGCCTATTAAATACCTATTATCACTATTTGGTTTCAA
ATTTGTGACACAGAAGAGCATAGTTAGAAATACTTGCAAAGCCTAGAATCATGA
ACTCATTTAAACCTTGCCCTGAAATGTTTCTTTTTGAATTGAGTTATTTTACACAT
GAATGGACAGTTACCATTATATATCTGAATCATTTCACATTCCCTCCCATGGCCT
AACAACAGTTTATCTTCTTATTTTGGGCACAACAGATGTCAGAGAGCCTGCTTTA
GGAATTCTAAGTAGAACTGTAATTAAGCAATGCAAGGCACGTACGTTTACTATG
TCATTGCCTATGGCTATGAAGTGCAAATCCTAACAGTCCTGCTAATACTTTTCTA
ACATCCATCATTTCTTTGTTTTCAGGGTCCAAACCTTGTCACTAGATGCAAAGAC
GCCTTAGCCGGAAGCGGCGCCACCAATTTCAGCCTGCTGAAACAGGCCGGCGAC
GTGGAAGAGAACCCTGGCCCTCCTGAGCAGGAGAGACAGATCACAGCCAGAGA
AGGGGCCAGTCGGAAAATCTTATCTAAGCTTTCTTTGCCTACCCGTGCCTGGGA
ACCAGCAATGAAGAAGAGTTTTGCTTTTGACAATGTTGGCTATGAAGGTGGTCT
GGATGGCCTGGGCCCTTCTTCTCAGCCGCAGAAGTGCTTCTTACAGATCAAAGG
CATGACCTGTGCATCCTGTGTGTCTAACATAGAAAGGAATCTGCAGAAAGAAGC
TGGTGTTCTCTCCGTGTTGGTTGCCTTGATGGCAGGAAAGGCAGAGATCAAGTA
TGACCCAGAGGTCATCCAGCCCCTCGAGATAGCTCAGTTCATCCAGGACCTGGG
TTTTGAGGCAGCAGTCATGGAGGACTACGCAGGCTCCGATGGCAACATTGAGCT
GACAATCACAGGGATGACCTGCGCGTCCTGTGTCCACAACATAGAGTCCAAACT
CACGAGGACAAATGGCATCACTTATGCCTCCGTTGCCCTTGCCACCAGCAAAGC
CCTTGTTAAGTTTGACCCGGAAATTATCGGTCCACGGGATATTATCAAAATTATT
GAGGAAATTGGCTTTCATGCTTCCCTGGCCCAGAGAAACCCCAACGCTCATCAC
TTGGACCACAAGATGGAAATAAAGCAGTGGAAGAAGTCTTTCCTGTGCAGCCTG
GTGTTTGGCATCCCTGTCATGGCCTTAATGATCTATATGCTGATACCCAGCAACG
AGCCCCACCAGTCCATGGTCCTGGACCACAACATCATTCCAGGACTGTCCATTC
TAAATCTCATCTTCTTTATCTTGTGTACCTTTGTCCAGCTCCTCGGTGGGTGGTAC
TTCTACGTTCAGGCCTACAAATCTCTGAGACACAGGTCAGCCAACATGGACGTG
CTCATCGTCCTGGCCACAAGCATTGCTTATGTTTATTCTCTGGTCATCCTGGTGG
TTGCTGTGGCTGAGAAGGCGGAGAGGAGCCCTGTGACATTCTTCGACACGCCCC
CCATGCTCTTTGTGTTCATTGCCCTGGGCCGGTGGCTGGAACACTTGGCAAAGA
GCAAAACCTCAGAAGCCCTGGCTAAACTCATGTCTCTCCAAGCCACAGAAGCCA
CCGTTGTGACCCTTGGTGAGGACAATTTAATCATCAGGGAGGAGCAAGTCCCCA
TGGAGCTGGTGCAGCGGGGCGATATCGTCAAGGTGGTCCCTGGGGGAAAGTTTC
CAGTGGATGGGAAAGTCCTGGAAGGCAATACCATGGCTGATGAGTCCCTCATCA
CAGGAGAAGCCATGCCAGTCACTAAGAAACCCGGAAGCACTGTAATTGCGGGG
TCTATAAATGCACATGGCTCTGTGCTCATTAAAGCTACCCACGTGGGCAATGAC
ACCACTTTGGCTCAGATTGTGAAACTGGTGGAAGAGGCTCAGATGTCAAAGGCA
CCCATTCAGCAGCTGGCTGACCGGTTTAGTGGATATTTTGTCCCATTTATCATCA
TCATGTCAACTTTGACGTTGGTGGTATGGATTGTAATCGGTTTTATCGATTTTGG
TGTTGTTCAGAGATACTTTCCTAACCCCAACAAGCACATCTCCCAGACAGAGGT
GATCATCCGGTTTGCTTTCCAGACGTCCATCACGGTGCTGTGCATTGCCTGCCCC
TGCTCCCTGGGGCTGGCCACGCCCACGGCTGTCATGGTGGGCACCGGGGTGGCC
GCGCAGAACGGCATCCTCATCAAGGGAGGCAAGCCCCTGGAGATGGCGCACAA
GATAAAGACTGTGATGTTTGACAAGACTGGCACCATTACCCATGGCGTCCCCAG
GGTCATGCGGGTGCTCCTGCTGGGGGATGTGGCCACACTGCCCCTCAGGAAGGT
TCTGGCTGTGGTGGGGACTGCGGAGGCCAGCAGTGAACACCCCTTGGGCGTGGC
AGTCACCAAATACTGTAAAGAGGAACTTGGAACAGAGACCTTGGGATACTGCA
CGGACTTCCAGGCAGTGCCAGGCTGTGGAATTGGGTGCAAAGTCAGCAACGTG
GAAGGCATCCTGGCCCACAGTGAGCGCCCTTTGAGTGCACCGGCCAGTCACCTG
AATGAGGCTGGCAGCCTTCCCGCAGAAAAAGATGCAGTCCCCCAGACCTTCTCT
GTGCTGATTGGAAACCGTGAGTGGCTGAGGCGCAACGGTTTAACCATTTCTAGC
GATGTCAGTGACGCTATGACAGACCACGAGATGAAAGGACAGACAGCCATCCT
GGTGGCTATTGACGGTGTGCTCTGTGGGATGATCGCAATCGCAGACGCTGTCAA
GCAGGAGGCTGCCCTGGCTGTGCACACGCTGCAGAGCATGGGTGTGGACGTGGT
TCTGATCACGGGGGACAACCGGAAGACAGCCAGAGCTATTGCCACCCAGGTTG
GCATCAACAAAGTCTTTGCAGAGGTGCTGCCTTCGCACAAGGTGGCCAAGGTCC
AGGAGCTCCAGAATAAAGGGAAGAAAGTCGCCATGGTGGGGGATGGGGTCAAT
GACTCCCCGGCCTTGGCCCAGGCAGACATGGGTGTGGCCATTGGCACCGGCACG
GATGTGGCCATCGAGGCAGCCGACGTCGTCCTTATCAGAAATGATTTGCTGGAT
GTGGTGGCTAGCATTCACCTTTCCAAGAGGACTGTCCGAAGGATACGCATCAAC
CTGGTCCTGGCACTGATTTATAACCTGGTTGGGATACCCATTGCAGCAGGTGTCT
TCATGCCCATCGGCATTGTGCTGCAGCCCTGGATGGGCTCAGCGGCCATGGCAG
CCTCCTCTGTGTCTGTGGTGCTCTCATCCCTGCAGCTCAAGTGCTATAAGAAGCC
TGACCTGGAGAGGTATGAGGCACAGGCGCATGGCCACATGAAGCCCCTGACGG
CATCCCAGGTCAGTGTGCACATAGGCATGGATGACAGGTGGCGGGACTCCCCCA
GGGCCACACCATGGGACCAGGTCAGCTATGTCAGCCAGGTGTCGCTGTCCTCCC
TGACGTCCGACAAGCCATCTCGGCACAGCGCTGCAGCAGACGATGATGGGGAC
AAGTGGTCTCTGCTCCTGAATGGCAGGGATGAGGAGCAGTACATCTAAACACAT
CACAACCACAACCTTCTCAGGTAACTATACTTGGGACTTAAAAAACATAATCAT
AATCATTTTTCCTAAAACGATCAAGACTGATAACCATTTGACAAGAGCCATACA
GACAAGCACCAGCTGGCACTCTTAGGTCTTCACGTATGGTCATCAGTTTGGGTTC
CATTTGTAGATAAGAAACTGAACATATAAAGGTCTAGGTTAATGCAATTTACAC
AAAAGGAGACCAAACCAGGGAGAGAAGGAACCAAAATTAAAAATTCAAACCA
GAGCAAAGGAGTTAGCCCTGGTTTTGCTCTGACTTACATGAACCACTATGTGGA
GTCCTCCATGTTAGCCTAGTCAAGCTTATCCTCTGGATGAAGTTGAAACCATATG
AAGGAATATTTGGGGGGTGGGTCAAAACAGTTGTGTATCAATGATTCCATGTGG
TTTGACCCAATCATTCTGTGAATCCATTTCAACAGAAGATACAACGGGTTCTGTT
TCATAATAAGTGATCCACTTCCAAATTTCTGATGTGCCCCATGCTAAGCTTTAAC
AGAATTTATCTTCTTATGACAAAGCAGCCTCCTTTGAAAATATAGCCAACTGCA
CACAGCTATG
SEQ ID NO: 39:
GCATGTTTGGTTAGGCTACGGCTTAGGGATTTATATATCAAAGGAGACTTTGTA
CAAGTGGGACAGGGATCTTATTTTACAAACAATTGTCTTACAAAATGAATAAAA
TAACACTTTGTTTTTATCTCCTGCTCTATTGTGCCATACTATTAAACGTTTATAAT
GCCCGTTCTGTTTCCAAATTTGTGATACTTATGAATATTAATAGGAATATTTGTA
AGGCCTAAAATATTTTGATTATGAAATCAAAACATTAATTTATTTAAACATTTTC
ATGAAAAGTGGTGGTTTGTGGTTTAGTTGATTTTATAGATTAGTGGGAGAATTTA
CATTCAAATGTCTAAATCACTTAAAATTGCCCCTTATGGCCTGACAGTATTTTTT
TTTAATTCCTTTGGGAACAACTATGTCCGTGAGCTTCCATCCAGAGATTATAGTA
GTAAATTGGAATTAAAGGATATGATGCACGTGAAATCACTTTGCAATCATCAAT
AGCTTCATAAATGTTAATTTTGTATCCTAATAGTAATGCTAATATTTTCCTAACA
TCTGTCATGTCTTTGTATTCAGGGTCCAAAATTTGTTGCTGCAAGTCAAGCTGCC
TTAGCCGGCAGCGGCGCCACCAACTTCAGCCTGCTGAAACAGGCCGGCGACGTG
GAAGAGAACCCTGGCCCTCCTGAGCAGGAGAGACAGATCACAGCCAGAGAAGG
GGCCAGTCGGAAAATCTTATCTAAGCTTTCTTTGCCTACCCGTGCCTGGGAACCA
GCAATGAAGAAGAGTTTTGCTTTTGACAATGTTGGCTATGAAGGTGGTCTGGAT
GGCCTGGGCCCTTCTTCTCAGCCGCAGAAGTGCTTCTTACAGATCAAAGGCATG
ACCTGTGCATCCTGTGTGTCTAACATAGAAAGGAATCTGCAGAAAGAAGCTGGT
GTTCTCTCCGTGTTGGTTGCCTTGATGGCAGGAAAGGCAGAGATCAAGTATGAC
CCAGAGGTCATCCAGCCCCTCGAGATAGCTCAGTTCATCCAGGACCTGGGTTTT
GAGGCAGCAGTCATGGAGGACTACGCAGGCTCCGATGGCAACATTGAGCTGAC
AATCACAGGGATGACCTGCGCGTCCTGTGTCCACAACATAGAGTCCAAACTCAC
GAGGACAAATGGCATCACTTATGCCTCCGTTGCCCTTGCCACCAGCAAAGCCCT
TGTTAAGTTTGACCCGGAAATTATCGGTCCACGGGATATTATCAAAATTATTGA
GGAAATTGGCTTTCATGCTTCCCTGGCCCAGAGAAACCCCAACGCTCATCACTT
GGACCACAAGATGGAAATAAAGCAGTGGAAGAAGTCTTTCCTGTGCAGCCTGG
TGTTTGGCATCCCTGTCATGGCCTTAATGATCTATATGCTGATACCCAGCAACGA
GCCCCACCAGTCCATGGTCCTGGACCACAACATCATTCCAGGACTGTCCATTCT
AAATCTCATCTTCTTTATCTTGTGTACCTTTGTCCAGCTCCTCGGTGGGTGGTACT
TCTACGTTCAGGCCTACAAATCTCTGAGACACAGGTCAGCCAACATGGACGTGC
TCATCGTCCTGGCCACAAGCATTGCTTATGTTTATTCTCTGGTCATCCTGGTGGT
TGCTGTGGCTGAGAAGGCGGAGAGGAGCCCTGTGACATTCTTCGACACGCCCCC
CATGCTCTTTGTGTTCATTGCCCTGGGCCGGTGGCTGGAACACTTGGCAAAGAG
CAAAACCTCAGAAGCCCTGGCTAAACTCATGTCTCTCCAAGCCACAGAAGCCAC
CGTTGTGACCCTTGGTGAGGACAATTTAATCATCAGGGAGGAGCAAGTCCCCAT
GGAGCTGGTGCAGCGGGGCGATATCGTCAAGGTGGTCCCTGGGGGAAAGTTTCC
AGTGGATGGGAAAGTCCTGGAAGGCAATACCATGGCTGATGAGTCCCTCATCAC
AGGAGAAGCCATGCCAGTCACTAAGAAACCCGGAAGCACTGTAATTGCGGGGT
CTATAAATGCACATGGCTCTGTGCTCATTAAAGCTACCCACGTGGGCAATGACA
CCACTTTGGCTCAGATTGTGAAACTGGTGGAAGAGGCTCAGATGTCAAAGGCAC
CCATTCAGCAGCTGGCTGACCGGTTTAGTGGATATTTTGTCCCATTTATCATCAT
CATGTCAACTTTGACGTTGGTGGTATGGATTGTAATCGGTTTTATCGATTTTGGT
GTTGTTCAGAGATACTTTCCTAACCCCAACAAGCACATCTCCCAGACAGAGGTG
ATCATCCGGTTTGCTTTCCAGACGTCCATCACGGTGCTGTGCATTGCCTGCCCCT
GCTCCCTGGGGCTGGCCACGCCCACGGCTGTCATGGTGGGCACCGGGGTGGCCG
CGCAGAACGGCATCCTCATCAAGGGAGGCAAGCCCCTGGAGATGGCGCACAAG
ATAAAGACTGTGATGTTTGACAAGACTGGCACCATTACCCATGGCGTCCCCAGG
GTCATGCGGGTGCTCCTGCTGGGGGATGTGGCCACACTGCCCCTCAGGAAGGTT
CTGGCTGTGGTGGGGACTGCGGAGGCCAGCAGTGAACACCCCTTGGGCGTGGC
AGTCACCAAATACTGTAAAGAGGAACTTGGAACAGAGACCTTGGGATACTGCA
CGGACTTCCAGGCAGTGCCAGGCTGTGGAATTGGGTGCAAAGTCAGCAACGTG
GAAGGCATCCTGGCCCACAGTGAGCGCCCTTTGAGTGCACCGGCCAGTCACCTG
AATGAGGCTGGCAGCCTTCCCGCAGAAAAAGATGCAGTCCCCCAGACCTTCTCT
GTGCTGATTGGAAACCGTGAGTGGCTGAGGCGCAACGGTTTAACCATTTCTAGC
GATGTCAGTGACGCTATGACAGACCACGAGATGAAAGGACAGACAGCCATCCT
GGTGGCTATTGACGGTGTGCTCTGTGGGATGATCGCAATCGCAGACGCTGTCAA
GCAGGAGGCTGCCCTGGCTGTGCACACGCTGCAGAGCATGGGTGTGGACGTGGT
TCTGATCACGGGGGACAACCGGAAGACAGCCAGAGCTATTGCCACCCAGGTTG
GCATCAACAAAGTCTTTGCAGAGGTGCTGCCTTCGCACAAGGTGGCCAAGGTCC
AGGAGCTCCAGAATAAAGGGAAGAAAGTCGCCATGGTGGGGGATGGGGTCAAT
GACTCCCCGGCCTTGGCCCAGGCAGACATGGGTGTGGCCATTGGCACCGGCACG
GATGTGGCCATCGAGGCAGCCGACGTCGTCCTTATCAGAAATGATTTGCTGGAT
GTGGTGGCTAGCATTCACCTTTCCAAGAGGACTGTCCGAAGGATACGCATCAAC
CTGGTCCTGGCACTGATTTATAACCTGGTTGGGATACCCATTGCAGCAGGTGTCT
TCATGCCCATCGGCATTGTGCTGCAGCCCTGGATGGGCTCAGCGGCCATGGCAG
CCTCCTCTGTGTCTGTGGTGCTCTCATCCCTGCAGCTCAAGTGCTATAAGAAGCC
TGACCTGGAGAGGTATGAGGCACAGGCGCATGGCCACATGAAGCCCCTGACGG
CATCCCAGGTCAGTGTGCACATAGGCATGGATGACAGGTGGCGGGACTCCCCCA
GGGCCACACCATGGGACCAGGTCAGCTATGTCAGCCAGGTGTCGCTGTCCTCCC
TGACGTCCGACAAGCCATCTCGGCACAGCGCTGCAGCAGACGATGATGGGGAC
AAGTGGTCTCTGCTCCTGAATGGCAGGGATGAGGAGCAGTACATCTAAAAACAT
CACAATTAAGAACATCTCAGGTAACTATATTTTGAATTTTTTAAAAAAGTAACT
ATAACAGTTATTATTAAAATAGCAAAGATTGACTGACGATTTCCAAGAGCCATA
CAGACCAGCACCAACCACTATTCTAAACTATTTATATATGTACATATTAGCTTTT
AAAATTCTCAAAATAGTTGCTGAGTTGGGAACCACTATTATTTCTATTTTGTAAA
TGAGAAAATGAAGATAAACATCAAAGCATAGGTTAAATAATTTTCCAAAGGGT
CAAAATTCAAAATTCAAACCAAAGTTTCAGTGTTGCCCATTGTCCTATTTTGACT
TATATGATGTGGCACACAGAGCCATCCAAGTAAGTGATGGCTCAGCAGGAGAA
TACTCTAGGAATTAGACTGAACCATATGTAAGAGCGCTTTATAGGACAAAAACA
GTTGAATATCAATGATTTCACATGGATCAACCTAATAGTTCAACTCATCCTTTCC
GTTGGAGAATATGATGGATCTACCTTCTGTGAACTTTATAGTGAACAATCTGCTA
TTACATTTTCAATTTGTCAACATGCTGAACTTTAATAGGACTTATTTTCTTATGAC
AAAA
SEQ ID NO: 40:
TGCTTCTCGACTGAGGTCAGAAACGTTTTTGCATTTTGACGATGTTCAGTTTCCA
TTTTCTGTGCACGTGGTCAGGTGTAGCTCTCTGGAACTCACACACTGAATAACTC
CACCAATCTAGATGTTGTTCTCTACGTAACTGTAATAGAAACTGACTTACGTAGC
TTTTAATTTTTATTTTCTGCCACACTGCTGCCTATTAAATACCTATTATCACTATT
TGGTTTCAAATTTGTGACACAGAAGAGCATAGTTAGAAATACTTGCAAAGCCTA
GAATCATGAACTCATTTAAACCTTGCCCTGAAATGTTTCTTTTTGAATTGAGTTA
TTTTACACATGAATGGACAGTTACCATTATATATCTGAATCATTTCACATTCCCT
CCCATGGCCTAACAACAGTTTATCTTCTTATTTTGGGCACAACAGATGTCAGAG
AGCCTGCTTTAGGAATTCTAAGTAGAACTGTAATTAAGCAATGCAAGGCACGTA
CGTTTACTATGTCATTGCCTATGGCTATGAAGTGCAAATCCTAACAGTCCTGCTA
ATACTTTTCTAACATCCATCATTTCTTTGTTTTCAGGGTCCAAACCTTGTCACTAG
ATGCAAAGACGCCTTAGCCGGAAGCGGCGCCACCAATTTCAGCCTGCTGAAACA
GGCCGGCGACGTGGAAGAGAACCCTGGCCCTCCTGAACAGGAGAGACAGGTCA
CAGCCAAAGAGGCCAGTCGGAAAATCTTATCTAAACTTGCTTTGCCCGGCCGGC
CCTGGGAGCAATCAATGAAGCAGAGCTTCGCCTTCGACAATGTTGGCTACGAAG
GGGGTCTGGACAGCACCAGCTCGTCCCCATCACAGAAGTGCTTCGTACAGATCA
AAGGCATGACCTGTGCGTCCTGTGTGTCTAACATAGAAAGGAGTCTCCAGAGAC
ATGCTGGTATTCTCTCAGTGTTGGTCGCCTTGATGTCGGGAAAGGCAGAGGTCA
AGTATGATCCGGAGATCATCCAGTCGCCCAGGATAGCTCAGCTCATCCAGGACC
TGGGCTTCGAAGCGTCAGTCATGGAGGACAACACAGTCTCTGAAGGTGACATCG
AACTGATTATCACAGGGATGACCTGTGCTTCCTGTGTCCACAACATAGAGTCCA
AGCTCACAAGGACAAATGGCATCACTTACGCCTCTGTGGCCCTTGCCACCAGCA
AAGCCCATGTGAAGTTCGATCCTGAAATTGTTGGTCCCCGTGACATCATCAAGA
TCATTGAGGAAATTGGCTTTCATGCTTCCCTGGCCCAGAGAAACCCCAACGCCC
ATCACTTGGACCACAAGACGGAAATAAAACAGTGGAAGAAGTCTTTCCTGTGCA
GCCTGGTGTTCGGCATCCCCGTCATGGGATTGATGGTCTACATGTTAATCCCCAG
CAGTACGCCTCAGGAGACGATGGTCCTGGACCACAACATCATCCCAGGACTGTC
CGTTCTCAATCTCATCTTCTTCATCTTGTGTACCTTTGTCCAATTTCTGGGTGGGT
GGTACTTCTACGTACAAGCCTACAAATCGCTGAGACACAGGTCCGCCAACATGG
ACGTACTCATCGTGCTCGCCACAACCATTGCCTATGCCTACTCCCTGGTCATCCT
GGTGGTCGCCGTAGCCGAGAAGGCAGAGAAGAGCCCCGTGACCTTCTTTGACAC
GCCCCCCATGCTCTTTGTGTTCATCGCCCTGGGACGGTGGCTGGAACACGTGGC
CAAGAGCAAAACTTCAGAAGCCCTTGCAAAACTCATGTCACTCCAAGCCACAGA
AGCCACAGTCGTGACCCTGGGTGAGGACAACTTAATCCTCAGAGAGGAGCAGG
TGCCCATGGAGCTGGTGCAGCGAGGCGACGTCATCAAGGTTGTCCCTGGGGGCA
AGTTCCCAGTGGATGGGAAAGTCCTCGAAGGCAATACCATGGCTGATGAGTCCC
TCATCACAGGAGAGGCCATGCCTGTCACTAAGAAACCTGGGAGCATAGTGATTG
CTGGCTCTATTAATGCTCATGGCTCTGTGCTCCTTAAAGCTACCCATGTGGGTAA
TGACACAACTTTGGCTCAGATTGTAAAGTTGGTGGAAGAGGCCCAGATGTCAAA
GGCTCCCATTCAGCAGCTGGCTGACCGGTTCAGTGGATATTTTGTCCCATTCATC
ATCATCATTTCAACCTTGACCCTGGTGGTGTGGATCGTCATTGGCTTTGTCGATT
TCGGTGTGGTTCAGAAGTACTTTCCTAGCCCTAGCAAGCACATCTCGCAGACAG
AGGTGATCATCCGCTTTGCCTTCCAGACGTCCATCACTGTGCTGTGCATCGCCTG
CCCCTGCTCCCTGGGGCTGGCCACACCCACAGCAGTCATGGTGGGCACTGGGGT
GGCTGCCCAGAACGGTGTCCTAATCAAAGGAGGGAAGCCTCTGGAGATGGCAC
ACAAGATAAAGACCGTTATGTTTGACAAAACGGGCACCATCACCCACGGGGTCC
CCAGAGTCATGCGGTTCCTGCTGCTCGCAGACGTGGCCACACTCCCCCTCAGGA
AGGTTCTGGCCGTGGTGGGCACCGCGGAGGCCAGCAGCGAGCACCCCTTAGGC
GTGGCCGTCACTAAATACTGCAAAGAGGAACTTGGGACGGAGACCCTGGGATA
CAGCACAGACTTCCAGGCAGTGCCCGGCTGTGGAATTAGCTGCAAAGTTAGCAA
CGTGGAGGGCATCCTGGCTCGCAGTGATCTGACTGCTCACCCTGTTGGAGTTGG
CAACCCTCCCACAGGGGAAGGTGCAGGTCCCCAGACCTTCTCCGTGCTGATTGG
AAACCGGGAATGGATGCGGCGAAACGGTTTAACCATCTCCAGTGACATCAGTG
ACGCCATGACAGATCACGAGATGAAAGGACAGACGGCCATCCTGGTGGCCATT
GATGGTGTGCTCTGCGGGATGATCGCCATCGCAGATGCTGTCAAACCAGAGGCT
GCCCTGGCTATCTACACCCTGAAAAGCATGGGTGTGGATGTGGCTCTGATCACA
GGGGACAACCGGAAGACAGCCAGAGCCATTGCTACTCAGGTTGGCATCAACAA
AGTCTTTGCGGAGGTACTGCCTTCTCACAAGGTGGCCAAGGTCCAGGAGCTTCA
GAATGAAGGGAAGAAAGTCGCCATGGTGGGAGATGGGGTGAATGACTCCCCAG
CCCTGGCCCAGGCTGACGTGGGCATCGCCATCGGGACTGGCACAGATGTTGCCA
TCGAAGCAGCAGACGTGGTCCTGATCAGAAATGACTTATTGGACGTCGTGGCCA
GCATTCATCTCTCCAAGAGGACCGTCCGGAGGATCCGGGTCAATCTGGTGCTGG
CATTGATTTATAACATGGTTGGGATACCTATTGCTGCAGGTGTCTTCATGCCCAT
TGGCATCGTGCTGCAGCCGTGGATGGGCTCAGCAGCCATGGCTGCGTCCTCTGT
CTCTGTGGTGCTCTCGTCTCTTCAGCTCAAGTGCTATAGAAAGCCCGACCTAGAG
AGATATGAGGCCCAGGCCCACGGCCGCATGAAGCCCCTGAGTGCCTCCCAAGTC
AGCGTGCACATTGGCATGGATGACCGGCGTCGGGATTCTCCCAGGGCCACCGCG
TGGGACCAGGTCAGCTACGTGAGCCAAGTGTCTCTGTCCTCCCTGACGTCAGAC
AGATTGTCTCGGCATGGGGGGCAGCAGAGGACGGTGGCGACAAATGGTCCCT
GCTCCTGAGTGACAGGGATGAAGAGCAGTGCATCTGATAAACACATCACAACC
ACAACCTTCTCAGGTAACTATACTTGGGACTTAAAAAACATAATCATAATCATT
TTTCCTAAAACGATCAAGACTGATAACCATTTGACAAGAGCCATACAGACAAGC
ACCAGCTGGCACTCTTAGGTCTTCACGTATGGTCATCAGTTTGGGTTCCATTTGT
AGATAAGAAACTGAACATATAAAGGTCTAGGTTAATGCAATTTACACAAAAGG
AGACCAAACCAGGGAGAGAAGGAACCAAAATTAAAAATTCAAACCAGAGCAA
AGGAGTTAGCCCTGGTTTTGCTCTGACTTACATGAACCACTATGTGGAGTCCTCC
ATGTTAGCCTAGTCAAGCTTATCCTCTGGATGAAGTTGAAACCATATGAAGGAA
TATTTGGGGGGTGGGTCAAAACAGTTGTGTATCAATGATTCCATGTGGTTTGAC
CCAATCATTCTGTGAATCCATTTCAACAGAAGATACAACGGGTTCTGTTTCATAA
TAAGTGATCCACTTCCAAATTTCTGATGTGCCCCATGCTAAGCTTTAACAGAATT
TATCTTCTTATGACAAAGCAGCCTCCTTTGAAAATATAGCCAACTGCACACAGC
TATGTTGATCA

In some embodiments, the present disclosure provides a recombinant AAV construct comprising: a polynucleotide cassette comprising: an expression cassette comprising a nucleic acid sequence having 80% homology to SEQ ID NO. 15 encoding a truncated form of ATP7B and a P2A encoding nucleic acid sequence having 80% sequence identity to SEQ ID NO. 17, positioned 5′ or 3′ to the nucleic acid sequence encoding a truncated form of ATP7B. In some embodiments, the polynucleotide cassette further comprises a nucleic acid homology sequence (e.g., a third nucleic acid sequence) positioned 5′ to the expression cassette and a second nucleic acid homology sequence (e.g., a fourth nucleic acid sequence) 3′ to the expression cassette. In some embodiments, the first nucleic acid homology sequence has a different number of base pairs than the second nucleic acid homology sequence. In some embodiments, the recombinant AAV construct further comprises AAV ITRs. In some embodiments, an AAV ITR has 90%, 95%, 99%, 100% sequence identity to one of SEQ ID Nos. 29-32.

In some embodiments, a provided composition comprises a polynucleotide cassette comprising a sequence selected from SEQ ID Nos. 36-42. In some embodiments, a provided composition comprises a polynucleotide cassette consisting of a sequence selected from SEQ ID NOs. 36-42.

### EXEMPLARY EMBODIMENTS

1. A composition comprising:
   a polynucleotide cassette comprising:
      an expression cassette comprising a first nucleic acid sequence and a second nucleic acid sequence, wherein the first nucleic acid sequence encodes a transgene; and the second nucleic acid sequence is positioned 5′ or 3′ to the first nucleic acid sequence and promotes the production of two independent gene products upon integration into a target integration site in the genome of a cell;
   a third nucleic acid sequence positioned 5′ to the expression cassette and comprising a sequence that is substantially homologous to a genomic sequence 5′ of a target integration site in a genome of a cell; and
   a fourth nucleic acid sequence positioned 3′ to expression cassette and comprising a sequence that is substantially homologous to a genomic sequence 3′ of a target integration site in the genome of the cell.
2. The composition of embodiment 1, wherein the composition further comprises a delivery vehicle.
3. The composition of embodiment 2, wherein the delivery vehicle comprises a lipid nanoparticle.
4. The composition of embodiment 3, wherein the delivery vehicle comprises a recombinant viral vector.
5. The composition of embodiment 4, wherein the recombinant viral vector is a recombinant adeno-associated (AAV) viral vector.
6. The composition of embodiment 5, wherein the recombinant viral vector is or comprises a capsid protein comprising an amino acid sequence having at least 95% sequence identity with the amino acid sequence of, AAV8, AAV-DJ; AAV-LK03; AAV-sL65; or AAV-NP59.
7. The composition of any one of the above embodiments, wherein the transgene is or comprises an ATP7B transgene.
8. The composition of embodiment 7, wherein the ATP7B transgene is a wt human ATP7B; a codon optimized ATP7B; a synthetic ATP7B; a ATP7B variant; a truncated form of ATP7B; a ATP7B mutant, or a ATP7B fragment.
9. The composition of embodiment 7, wherein the ATP7B transgene has 80% sequence identity to SEQ ID NO. 14 or SEQ ID NO. 15.
10. The composition of any one of the above embodiments, wherein the composition further comprises AAV2 ITR sequences.
11. The composition of any one of the above embodiments, wherein the polynucleotide cassette does not comprise a promoter sequence.
12. The composition of any one of the above embodiments, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
13. The composition of any one of the above embodiments, wherein the third and fourth nucleic acid sequences are homology arms that integrate the expression cassette at a target integration site comprising an endogenous promoter and an endogenous gene.
14. The composition of embodiment 13, wherein the target integration site is the endogenous albumin gene locus.
15. A method of integrating a transgene into the genome of at least a population of cells in a tissue in a subject, said method comprising
   administering to a subject a composition comprising:
      a polynucleotide cassette comprising:
   an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell;
a third nucleic acid sequence positioned 5′ to the expression cassette and comprising a sequence that is substantially homologous to a genomic sequence 5′ of the target integration site in the genome of the cell; and
a fourth nucleic acid sequence positioned 3′ to the expression and comprising a sequence that is substantially homologous to a genomic sequence 3′ of the target integration site in the genome of the cell;
wherein, after administering the composition, the transgene is integrated into the genome of the population of cells.
16. The method of embodiment 15, wherein the integration does not comprise exogenous nuclease activity.
17. The method of embodiment 15, wherein the composition further comprises a delivery vehicle.
18. The method of embodiment 17, wherein the delivery vehicle comprises a lipid nanoparticle.
19. The method of embodiment 17, wherein the delivery vehicle comprises a recombinant viral vector.
20. The method of embodiment 19, wherein the recombinant viral vector is a recombinant adeno-associated (AAV) viral vector.
21. The method of embodiment 19, wherein the recombinant viral vector is or comprises a capsid protein comprising an amino acid sequence having at least 95% sequence identity with the amino acid sequence of AAV8, AAV-DJ; AAV-LK03; AAV-sL65; or AAV-NP59.
22. The method of any one of embodiments 15-21, wherein the transgene is or comprises a copper-transporting ATPase 2 (ATP7B) transgene.
23. The method of embodiment 22, wherein the ATP7B transgene is a wt human ATP7B; a codon optimized ATP7B; a synthetic ATP7B; a ATP7B variant; a truncated form of ATP7B, a ATP7B mutant, or a ATP7B fragment.
24. The method of embodiment 22, wherein the ATP7B transgene has 80% sequence identity to SEQ ID NO. 14 or SEQ ID NO. 15.
25. The method of any one of embodiments 15-24, wherein the composition further comprises AAV2 ITR sequences.
26. The method of any one of embodiments 15-25, wherein the polynucleotide cassette does not comprise a promoter sequence.
27. The method of any one of embodiments 15-26, wherein upon integration of the expression cassette into the target integration site in the genome of the cell, the transgene is expressed under control of an endogenous promoter at the target integration site.
28. The method of embodiment 27, wherein the target integration site is the albumin locus comprising an endogenous albumin promoter and an endogenous albumin gene.
29. The method of embodiment 28, wherein upon integration of the expression cassette into the target integration site in the genome of a cell, the transgene is expressed under control of the endogenous albumin promoter without disruption of the endogenous albumin gene expression.
30. The method of any one of the above embodiments, wherein the tissue is the liver.
31. The method of any one of the above embodiments, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
32. The method of any one of the above embodiments, wherein the third and fourth nucleic acid sequences are homology arms that integrate the expression cassette at a target integration site comprising an endogenous promoter and an endogenous gene.
33. The method of embodiment 32, wherein the target integration site is the endogenous albumin gene locus.
34. A method of increasing a level of expression of a transgene in a tissue over a period of time, said method comprising
administering to a subject in need thereof a composition that delivers a transgene that integrates into the genome of at least a population of cells in the tissue of the subject, wherein the composition comprises:
   a polynucleotide cassette comprising
an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products upon integration into a target integration site in the genome of the cell;
   a third nucleic acid sequence positioned 5′ to the expression cassette and comprising a sequence that is substantially homologous to a genomic sequence 5′ of the target integration site in the genome of the cell; and
   a fourth nucleic acid sequence positioned 3′ to the expression cassette and comprising a sequence that is substantially homologous to a genomic sequence 3′ of the target integration site in the genome of the cell;
wherein, after administering the composition, the transgene is integrated into the genome of the population of cells and the level of expression of the transgene in the tissue increases over a period of time.
35. The method of embodiment 34, wherein the integration of the transgene does not comprise exogenous nuclease activity.
36. The method of embodiment 34 or 35, wherein the increased level of expression comprises an increased percent of cells in the tissue expressing the transgene.
37. The method of embodiment 34, wherein the composition further comprises a delivery vehicle.
38. The method of embodiment 37, wherein the delivery vehicle comprises a lipid nanoparticle.
39. The method of embodiment 37, wherein the delivery vehicle comprises a recombinant viral vector.
40. The method of embodiment 39, wherein the recombinant viral vector is a recombinant AAV vector.
41. The method of embodiment 40, wherein the recombinant viral vector is or comprises a capsid protein comprising an amino acid sequence having at least 95% sequence identity with the amino acid sequence of, AAV8, AAV-DJ; AAV-LK03; AAV-sL65; or AAV-NP59.
42. The method of any one of embodiments 34-41, wherein the transgene is or comprises a copper-transporting ATPase 2 (ATP7B) transgene.
43. The method of embodiment 42, wherein the ATP7B transgene is a wt human ATP7B; a codon optimized ATP7B; a synthetic ATP7B; a ATP7B variant; a truncated form of ATP7B; a ATP7B mutant, or a ATP7B fragment.
44. The method of embodiment 42, wherein the ATP7B transgene has 80% sequence identity to SEQ ID NO. 14 or SEQ ID NO. 15.
45. The method of any one of embodiments 34-44, wherein the composition further comprises AAV2 ITR sequences and/or ITR sequences selected from SEQ ID NO: 29-32.
46. The method of any one of embodiments 34-45, wherein the polynucleotide cassette does not comprise a promoter sequence.
47. The method of any one of embodiments 34-46, wherein upon integration of the expression cassette into the target integration site in the genome of the cell, the transgene is expressed under control of an endogenous promoter at the target integration site.
48. The method of embodiment 47, wherein the target integration site is the albumin locus comprising an endogenous albumin promoter and an endogenous albumin gene.
49. The method of embodiment 48, wherein upon integration of the expression cassette into the target integration site in the genome of a cell, the transgene is expressed under control of the endogenous albumin promoter without disruption of the endogenous albumin gene expression.
50. The method of any one of embodiments 34-49, wherein the tissue is the liver.
51. The method of any one of embodiments 34-50, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
52. The method of any one of embodiments 34-51, wherein the third and fourth nucleic acid sequences are homology arms that integrate the expression cassette at a target integration site comprising an endogenous promoter and an endogenous gene.
53. The method of embodiment 52, wherein the target integration site is the endogenous albumin gene locus.
54. A recombinant viral vector for integrating a transgene into a target integration site in the genome of a cell, comprising a polynucleotide cassette comprising:
(i) an expression cassette comprising a first nucleic acid sequence and a second nucleic acid sequence, wherein the first nucleic acid sequence comprises a ATP7B transgene; and the second nucleic acid sequence is positioned 5′ or 3′ to the first nucleic acid sequence and promotes the production of two independent gene products upon integration into the target integration site in the genome of the cell;
(ii) a third nucleic acid sequence positioned 5′ to the expression cassette and comprising a sequence that is substantially homologous to a genomic sequence 5′ of the target integration site in the genome of the cell; and
(iii) a fourth nucleic acid sequence positioned 3′ of the expression cassette and comprising a sequence that is substantially homologous to a genomic sequence 3′ of the target integration site in the genome of the cell.
55. The recombinant viral vector of embodiment 54, wherein the third nucleic acid is between 900-1150 nucleotides.
56. The recombinant viral vector of embodiment 54 or embodiment 55, wherein the fourth nucleic acid is between 1500-1750 nucleotides.
57. The recombinant viral vector of any one of embodiments 54-56, wherein the recombinant viral vector is a recombinant AAV vector.
58 The recombinant viral vector of embodiment 57, wherein the recombinant viral vector is or comprises a capsid protein comprising an amino acid sequence having at least 95% sequence identity with the amino acid sequence of AAV8, AAV-DJ; AAV-LK03; AAV-sL65; or AAV-NP59.
59. The recombinant viral vector of any one of embodiments 54-58, further comprising AAV2 ITR sequences and/or ITR sequences selected from SEQ ID NO: 29-32.
60. The recombinant viral vector of any one of embodiments 54-59, wherein the polynucleotide cassette does not comprise a promoter sequence.
61. The recombinant viral vector of any one of embodiments 54-60, wherein upon integration of the expression cassette into the target integration site in the genome of a cell, the ATP7B transgene is expressed under control of an endogenous promoter at the target integration site.
62. The recombinant viral vector of any one of embodiments 61, wherein the target integration site is the albumin locus comprising an endogenous albumin promoter and an endogenous albumin gene.
63. The recombinant viral vector of embodiment 61, wherein upon integration of the expression cassette into the target integration site in the genome of a cell, the ATP7B transgene is expressed under control of the endogenous albumin promoter without disruption of the endogenous albumin gene expression.
64. The recombinant viral vector of any one of embodiments 61, wherein the two independent gene products are a ATP7B protein expressed from the ATP7B transgene and a peptide comprising an endogenous protein expressed from an endogenous gene at the integration site.
65. The recombinant viral vector of any one of embodiments 54-64, wherein the cell is a liver cell.
66. The recombinant viral vector of any one of embodiments 54-65, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and a C-terminal intein splicing region; or
   d) a nucleic acid sequence encoding a splice donor and a splice acceptor.
67. The recombinant viral vector of any of embodiments 54-66, wherein the third and fourth nucleic acid sequences are homology arms that integrate the ATP7B transgene and the second nucleic acid sequence into an endogenous albumin gene locus comprising an endogenous albumin promoter and an endogenous albumin gene.
68. The recombinant viral vector of embodiment 67, wherein the third and fourth nucleic acid sequences are homology arms that integrate the ATP7B transgene and the second nucleic acid sequence into an endogenous albumin gene locus in frame with the endogenous albumin promoter and the endogenous albumin gene.
69. The recombinant viral vector of embodiment 67 or embodiment 68, wherein the homology arms direct integration of the polynucleotide cassette immediately 3′ of the start codon of the endogenous albumin gene or immediately 5′ of the stop codon of the endogenous albumin gene.
70. The recombinant viral vector of any one of embodiments 54-69, wherein the ATP7B transgene is a wt human ATP7B; a codon optimized ATP7B; a synthetic ATP7B; a ATP7B variant; a truncated form of ATP7B; a ATP7B mutant, or a ATP7B fragment.
71. A method comprising a step of
administering to a subject a dose of a composition that delivers to cells in a tissue of the subject a transgene, wherein the transgene (i) encodes ATP7B; (ii) integrates at a target integration site in the genome of a plurality of the cells; (iii) functionally expresses ATP7B once integrated; and (iv) confers a selective advantage to the plurality of cells relative to other cells in the tissue, so that, over time, the tissue achieves a level of functional expression of ATP7B that is greater than cells that did not integrate the transgene wherein the composition comprises:
   a polynucleotide cassette comprising:
      an expression cassette comprising a first nucleic acid sequence and a second nucleic acid sequence, wherein the first nucleic acid sequence encodes the transgene; and the second nucleic acid sequence is positioned 5′ or 3′ to the first nucleic acid sequence and promotes the production of two independent gene products when the transgene is integrated at the target integration site;
   a third nucleic acid sequence positioned 5′ to the expression cassette and comprising a sequence that is substantially homologous to a genomic sequence 5′ of the target integration site; and
   a fourth nucleic acid sequence positioned 3′ to the expression cassette and comprising a sequence that is substantially homologous to a genomic sequence 3′ of the target integration site.
72. The method of embodiment 71, wherein the integration of the transgene does not comprise exogenous nuclease activity.
73. The method of embodiment 71 or 72, wherein the selective advantage comprises an increased percent of cells in the tissue expressing the transgene.
74. The method of any one of embodiments 71-73, wherein the composition further comprises a delivery vehicle.
75. The method of embodiment 74, wherein the delivery vehicle comprises a lipid nanoparticle.
76. The method of embodiment 74, wherein the composition comprises a recombinant viral vector.
77. The method of embodiment 76, wherein the recombinant viral vector is a recombinant AAV vector.
78. The method of embodiment 77, wherein the recombinant viral vector is or comprises a capsid protein comprising an amino acid sequence having at least 95% sequence identity with the amino acid sequence of AAV8, AAV-DJ; AAV-LK03; AAV-sL65 or AAV-NP59.
79. The method of embodiment 71, wherein the ATP7B transgene is a wt human ATP7B; a codon optimized ATP7B; a synthetic ATP7B; a ATP7B variant; a truncated form of ATP7B; a ATP7B mutant, or a ATP7B fragment.
80. The method of any one of embodiments 71-79, wherein the composition further comprises AAV2 ITR sequences and/or ITR sequences selected from SEQ ID NO: 29-32.
81. The method of any one of embodiments 71-80, wherein the polynucleotide cassette does not comprise a promoter sequence.
82. The method of any one of embodiments 71-81, wherein upon integration of the expression cassette into the target integration site in the genome of the cell, the transgene is expressed under control of an endogenous promoter at the target integration site.
83. The method of embodiment 80, wherein the target integration site is the albumin locus comprising an endogenous albumin promoter and an endogenous albumin gene.
84. The method of embodiment 83, wherein upon integration of the polynucleotide cassette into the target integration site in the genome of a cell, the transgene is expressed under control of the endogenous albumin promoter without disruption of the endogenous albumin gene expression.
85. The method of any one of embodiments 71-84, wherein the tissue is the liver.
86. The method of any one of embodiments 71-85, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
87. The method of any one of embodiments 71-86, wherein the third and fourth nucleic acid sequences are homology arms that integrate the expression cassette at a target integration site comprising an endogenous promoter and an endogenous gene.
88. The method of embodiment 87, wherein the target integration site is the endogenous albumin locus.
89. A method of treating Wilson's Disease, the method comprising administering to a subject a dose of a composition comprising:
   a polynucleotide cassette comprising
      an expression cassette comprising a first nucleic acid sequence and a second nucleic acid sequence, wherein the first nucleic acid sequence encodes a ATP7B transgene; and the second nucleic acid sequence is positioned 5′ or 3′ to the first nucleic acid sequence and promotes the production of two independent gene products when the transgene is integrated at the target integration site;
   a third nucleic acid sequence positioned 5′ to the first nucleic acid sequence and comprising a sequence that is substantially homologous to a genomic sequence 5′ of the target integration site; and
   a fourth nucleic acid sequence positioned 3′ to the second nucleic acid sequence and comprising a sequence that is substantially homologous to a genomic sequence 3′ of the target integration site.
wherein, after administering the composition, the transgene is integrated into the genome of the population of cells.
90. The method of embodiment 89, wherein the integration does not comprise exogenous nuclease activity.
91. The method of embodiment 89, wherein the composition further comprises a delivery vehicle.
92. The method of embodiment 91, wherein the delivery vehicle comprises a lipid nanoparticle.
93. The method of embodiment 91, wherein the delivery vehicle comprises a recombinant viral vector.
94. The method of embodiment 93, wherein the recombinant viral vector is a recombinant adeno-associated (AAV) viral vector.
95. The method of embodiment 93, wherein the recombinant viral vector is or comprises a capsid protein comprising an amino acid sequence having at least 95% sequence identity with the amino acid sequence of AAV8, AAV-DJ; AAV-LK03; AAV-sL65; or AAV-NP59.
96. The method of embodiment 95, wherein the ATP7B transgene is a wt human ATP7B; a codon optimized ATP7B; a synthetic ATP7B; a ATP7B variant; a truncated form of ATP7B; a ATP7B mutant, or a ATP7B fragment.
97. The method of embodiment 95, wherein the ATP7B transgene has 80% sequence identity to SEQ ID NO. 14 or SEQ ID NO. 15.
98. The method of any one of embodiments 89-97, wherein the composition further comprises AAV2 ITR sequences and/or ITR sequences selected from SEQ ID NO: 29-32.
99. The method of any one of embodiments 89-98, wherein the polynucleotide cassette does not comprise a promoter sequence.
100. The method of any one of embodiments 89-99, wherein upon integration of the expression cassette into the target integration site in the genome of the cell, the transgene is expressed under control of an endogenous promoter at the target integration site.
101. The method of embodiment 100, wherein the target integration site is the albumin locus comprising an endogenous albumin promoter and an endogenous albumin gene.
102. The method of embodiment 101, wherein upon integration of the polynucleotide cassette into the target integration site in the genome of a cell, the transgene is expressed under control of the endogenous albumin promoter without disruption of the endogenous albumin gene expression.
103. The method of any one of embodiments 89-102, wherein the tissue is the liver.
104. The method of any one of embodiments 89-103, wherein the second nucleic acid sequence comprises:
   a) a nucleic acid sequence encoding a 2A peptide;
   b) a nucleic acid sequence encoding an internal ribosome entry site (IRES);
   c) a nucleic acid sequence encoding an N-terminal intein splicing region and C-terminal intein splicing region; or
   d) a nucleic acid sequence encoding a splice donor and a splice acceptor.
105. The method of any one of embodiments 89-104, wherein the third and fourth nucleic acid sequences are homology arms that integrate expression cassette into at a target integration site comprising an endogenous promoter and an endogenous gene.
106. The method of embodiment 105, wherein the target integration site is the endogenous albumin gene locus.
107. The method of embodiment 89, wherein the subject has received or is receiving treatment for Wilson's Disease.
108. The method of embodiment 107, wherein the subject has received or is receiving DPA and/or trientine (e.g., Syprine®) 109. The method of embodiment 107 or 108, wherein DPA and/or trientine and the composition are administered in combination.
110. The method of embodiment 109, wherein DPA and/or trientine and the composition are administered simultaneously, or sequentially.
111. The method of embodiment 89, wherein the subject receives a lower or reduced dose of the treatment the subject is receiving after treatment with the composition.
112. The method of embodiment 89, wherein the subject receives the same dose of the treatment the subject has received or is receiving after treatment with the composition.
113. The method of embodiment 89, wherein the subject stops receiving the treatment the subject has received or is receiving after treatment with the composition.
114. The method of embodiment 89, wherein the subject is between six months and 35 years old.
115. The method of embodiment 89, wherein the subject is one year, two years, three years, four years, or five years old.
116. A method of monitoring gene therapy, the method comprising a step of:
detecting, in a biological sample from a subject who has received gene therapy treatment comprising the composition of embodiment 1, a level or activity of a biomarker generated by integration of the integrating gene therapy treatment, as a surrogate for one or more characteristics of the status of the gene therapy treatment, wherein the one or more characteristics of the status of the gene therapy treatment is selected from the group consisting of level of a payload, activity of a payload, level of integration of the gene therapy treatment in a population of cells, and combinations thereof.
117. The method of embodiment 116, wherein the payload is or comprises a peptide expressed intracellularly.
118. The method of embodiment 116, wherein the payload is or comprises a peptide that is secreted extracellularly.
119. The method of any one of embodiments 116-118, wherein the payload is encoded by the polynucleotide cassette
120. The method of any one of embodiments 116-119, wherein the biological sample is or comprises hair, skin, feces, blood, plasma, serum, cerebrospinal fluid, urine, saliva, tears, vitreous humor, liver biopsy or mucus.
121. The method of any one of embodiments 116-120, wherein the step of detecting comprises an immunological assay or a nucleic acid amplification assay.
122. The method of any embodiments 116-121, wherein the biomarker comprises a detectable moiety that, after translation of the polypeptide encoded by the target site, becomes fused to the polypeptide encoded by the target site.
123. The method of any one of embodiments 116-123, wherein the biomarker comprises a detectable moiety that, after translation of the polypeptide encoded by the target site, becomes fused to the polypeptide encoded by the payload.
124. The method of any one of embodiments 116-123, wherein the biomarker comprises a detectable moiety that is a 2A peptide
125. The method of embodiment 124, wherein the 2A peptide is selected from the group consisting of P2A, T2A, E2A and F2A.
126. The method of any one of embodiments 116-125, wherein the subject receives a single dose of the gene therapy treatment or gene-integrating composition.
127. The method of any one of embodiments 116-126, wherein the detecting step is performed 1, 2, 3, 4, 5, 6, 7, 8 or more weeks after the subject has received the gene therapy treatment or gene-integrating composition.
128. The method of any one of embodiments 116-127, wherein the detecting step is performed at multiple time points after the subject has received the gene therapy treatment or gene-integrating composition.
129. The method of any one of embodiments 116-128, wherein the detecting step is performed over a period of at least 3 months after the subject has received the gene therapy treatment or gene-integrating composition.
130. The method of any one of embodiments 116-129, wherein the method further comprises monitoring the subject for autoimmune response to the gene therapy.
131. A composition comprising a recombinant AAV viral vector comprising:
   a polynucleotide cassette comprising:
      an expression cassette comprising a first nucleic acid sequence and a second nucleic acid sequence, wherein the first nucleic acid sequence has 80% sequence identity to SEQ ID NO. 15 and encodes a truncated form of human ATP7B; and the second nucleic acid sequence
      (i) is positioned 5′ or 3′ to the first nucleic acid sequence; and
      (ii) promotes the production of two independent gene products upon integration into a target integration site in the genome of a cell;
   a third nucleic acid sequence positioned 5′ to the expression cassette and comprising a sequence that is substantially homologous to a genomic sequence 5′ of a target integration site in a genome of a cell; and
   a fourth nucleic acid sequence positioned 3′ to expression cassette and comprising a sequence that is substantially homologous to a genomic sequence 3′ of a target integration site in the genome of the cell.
132. The composition of embodiment 131, wherein the recombinant viral vector is or comprises a capsid protein comprising an amino acid sequence having at least 95% sequence identity with the amino acid sequence of, AAV8, AAV-DJ; AAV-LK03; AAV-sL65; or AAV-NP59.
133. The composition of embodiments 131 or 132, wherein the composition further comprises AAV2 ITR sequences and/or ITR sequences selected from SEQ ID NO: 29-32.
134. The composition of embodiments 131-133, where the second nucleic acid sequence has 80% sequence identity to SEQ ID NO. 17.
135. The composition of embodiments 131-133, where the second nucleic acid sequence encodes a P2A peptide having 90% sequence identity to SEQ ID NO. 18.
136. A recombinant viral vector comprising SEQ ID NO: 34.
137. A recombinant viral vector comprising SEQ ID NO: 35.
138. A recombinant viral vector comprising SEQ ID NO: 36.
139. A recombinant viral vector comprising SEQ ID NO: 37.
140. A recombinant viral vector comprising SEQ ID NO: 38.
141. A recombinant viral vector comprising SEQ ID NO: 39.
142. A recombinant viral vector comprising SEQ ID NO: 40.
143. A composition comprising the recombinant viral vector of any one of embodiments 136-142.
144. A method of integrating a transgene into the genome of at least a population of cells in a tissue in a subject, said method comprising administering to a subject a composition comprising the recombinant viral vector of any one of embodiments 136-142.
145. A composition comprising the recombinant viral vector of embodiments 54-70.
146. A method of treatment comprising administering a composition of any one of embodiments 1, 143, or 145, wherein the composition is administered to a subject in dosages between 1E12 and 1E14 vg/kg.
147. The method of embodiment 146, wherein the composition is administered only once.
148. The method of embodiment 146, wherein the composition is administered more than once.
149. The method of any one of embodiments 146-148, wherein the subject is a newborn.
150. The method of any one of embodiments 146-148, wherein the subject is between 0 days and 1 month of age.
151. The method of any one of embodiments 146-148, wherein the subject is between 3 months of age and 1 year of age.
152. The method of any one of embodiments 146-148, wherein the subject is between 1 year of age and 5 years of age.
153. The method of any one of embodiments 146-148, wherein the subject is 5 years of age or older.

### EXEMPLIFICATION

### Example 1. Method and Materials

### Vector Production

Vectors were produced in HEK293 cells by transient transfection of plasmids containing vector genome, cDNA sequences encoding capsid and other helper proteins, followed by AAVX-affinity purification and CsCl gradient purification. Viral titer was determined by ddPCR. Vectors employed include AAV-DJ, AAV-LK03, and AAV-sL65 capsids.

### Animal Experiments

Animals were housed in plastic containers fitted to the Innorack® IVC Mouse 3.5 racks. Housing room temperature was maintained at 68° C. to 79° C. with relative humidity level of 30% to 70%. Animals had access to food and water ad libitum. Wilson's Disease (WD) mice (Jax Stock 001576) have a natural mutation in Atp7b cDNA G2135A, leading to amino acid change G712D and a deficient protein ATP7B (also known as Atp7b^{tx-J} mice). Healthy littermates (heterozygous [Het] or wild-type [wt]) served as controls. Chimeric PXB mice with a humanized liver were purchased from PhoenixBio Co Ltd. Mice were produced by xenotransplanting human hepatocytes into immunodeficient recipient cDNA-uPA+/−/SCID mice. Neonatal, juvenile or adult mice were intravenously injected with vehicle or vector via facial vein, retro orbital sinus, or lateral tail veins. Animals were monitored for health and survival daily. Euthanasia was performed for animals considered as moribund, displaying severe adverse signs including prostration, decreased motor activity, inability to right, cold to touch, pale, and/or tremors. Blood samples were collected periodically with an interval between 1 to 8 weeks by submandibular bleed, and terminal blood and tissues were collected at necropsies. 24-hour urine samples were collected from selected animals using metabolic chambers (Hatteras Instruments MMC100).

### Plasma Alanine Aminotransferase Activity Assay

Plasma alanine aminotransferase activity was quantified using an alanine aminotransferase activity colorimetric assay kit (BioVision) with 1:10 diluted plasma samples.

### Plasma ALB-2A Fusion Protein and Albumin Quantitation

ALB-2A was quantitated in mouse plasma samples in an enzyme-linked immunosorbent assay (ELISA) using a proprietary recombinant monoclonal rabbit anti-2A antibody for capture and an HRP-labeled anti-ALB polyclonal antibody for detection. Purified recombinant mouse or human ALB-2A were used as standards to build calibration curves from which ALB-2A concentrations were interpolated. Plasma human albumin level in the PXB mice was measured in a human-specific ELISA to monitor degree and durability of human hepatocyte engraftment.

### Targeted Genomic DNA Integration in Liver

Genomic DNA was extracted from frozen liver tissues and targeted genomic DNA integration was analyzed by long-range polymerase chain reaction (PCR) amplification, followed by quantitative polymerase chain reaction (qPCR) quantification using a qualified method. Long Range PCR was performed using a forward primer (F1) and a reverse primer (R1). PCR products were purified with solid phase reversible immobilization beads (ABM, G950) and used as template for qPCR using the forward primer (F1), a reverse primer (R2) and a probe (P1). Primers and probes for mouse experiments were (F1m) 5′-ATGTTCCACGAAGAAGCCA-3′, (R1m) 5′-TCAGCAGGCTGAAATTGGT-3, (R2m) 5′-AGCTGTTTCTTACTCCATTCTCA-3′, (P1m) 5′-AGGCAACGTCATGGGTGTGACTTT-3′. The mouse transferrin receptor (Tfrc) was used as an internal control in qPCR. The primers and probes for humanized mouse experiments are (F1h) 5′-GCTCTCCTGCCTGTTCTTTAG-3′, (R1h) 5′-TCAGCAGGCTGAAATTGGT-3, (R2h) 5′-TCAGCATAATAAGGGCAACACT-3′, (P1h) 5′-GCAAGAACTGTCAATTCAAGCTAGCAACT-3′. Human RNA pyrophosphohydrolase (RPPH) was used as an internal control in qPCR.

Fused mRNA in Liver

Total RNA was extracted from frozen liver tissues and transcripts from integrated transgenes were quantified by reverse transcription-coupled droplet digital polymerase chain reaction (ddPCR) using a qualified method with a forward primer (F2), a reverse primer (R3) and a probe (P2). Primers and probes for mouse experiments were (F2m) 5′-CACACTTCCAGAGAAGGAGAAGC-3′, (R3m) 5′-TCAGCAGGCTGAAGTTGGT-3′, (P2m) 5′-AAGACGCCTTAGCCGGCAGCGGC-3′. Primers and probes for humanized mouse experiments were (F2h) 5′-TGAGAAGGAGAGACAAATCAAGAA-3′, (R3h) 5′-TCGCCGGCCTGTTTCAG-3, (P2h) 5′-TTAGGCTTAGGAAGCGGCGC-3′.

### Protein Expression in Liver by Immunohistochemistry

ATP7B or ALB-2A protein expression in liver tissues was analyzed in formalin-fixed, paraffin-embedded tissue sections of 5 μm by immunohistochemistry analysis using rabbit polyclonal anti-ATP7B antibody (Abcam ab124973) and a proprietary anti-2A antibody, respectively.

### Copper Quantification

Copper in liver tissue or urine was quantified by ICP-MS.

### Data Processing and Analyses

Raw data were recorded and calculated when appropriate using Microsoft Excel. Graphs were generated and statistical analyses performed using Prism version 9 (GraphPad). Data in texts and graphs represent means and standard deviations unless noted. One-way analysis of variance, mixed-effects analysis with multiple comparison or two-sided Student's t-tests were performed to compare values between groups, where statistical significance was defined as P<0.05.

### Example 2. Viral Vector Compositions can Provide Durable Editing Activity In Vivo when Administered at Various Timepoints

The present example describes that, among other things, viral vector compositions comprising a sequence encoding ATP7B (e.g., truncated ATP7B) may be administered to a subject (e.g., a subject suffering from WD) at various timepoints in order to provide durable integration of an ATP7B gene sequence.

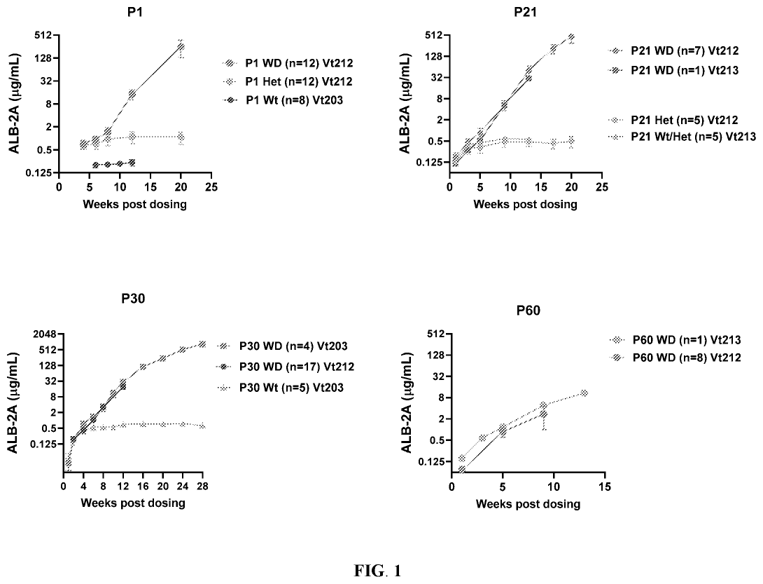

Viral vectors comprising an AAV-DJ viral capsid, truncated ATP7B (tATP7B) transgene, P2A sequence, and flanking homology arms were constructed (Table 2). ATP7B sequences were of mouse (mtATP7B) or human (htATP7B) origin. Homology arms comprised sequences of even (0.6/0.6 kb) or uneven (1.0/0.6 kB) length. 1×10¹⁴ vg/kg dose viral vector compositions were intravenously administered to homozygous WD mice, heterozygous mice, and wild-type mice at post-natal day 1 (P1), P21, P30, or P60. Mice were assessed for levels of ALB-2A, which served as a biomarker for levels of ATP7B integration in target cells (e.g., hepatocytes) (FIG. 1)

Among other things, the present disclosure demonstrates that treatment of a subject (e.g., a subject suffering from WD) with viral vectors comprising a sequence encoding a functional ATP7B protein (e.g., truncated ATP7B) may provide improved editing activity (e.g., increased rates of transgene integration) and durability as compared to a reference (e.g., control or vehicle-treated subjects). In some embodiments, treatment of a subject (e.g., a subject suffering from WD) with viral vectors comprising a sequence encoding a functional ATP7B protein (e.g., truncated ATP7B) may provide improved editing activity (e.g., increased rates of transgene integration) and durability as compared to a reference (e.g., control or vehicle-treated subjects). over a period of several weeks (e.g., at least 10 weeks, 15 weeks, 20 weeks, 25 weeks, 28 weeks, etc.) post-administration in target cells (e.g., hepatocytes) in vivo. In some embodiments, treatment of a subject (e.g., a subject suffering from WD) with viral vectors comprising a sequence encoding a functional ATP7B protein (e.g., truncated ATP7B) may provide improved editing activity (e.g., increased rates of transgene integration) and durability when administered at particular timepoints (e.g., P1, P21, P30, P60).

### Example 3. Viral Vector Compositions May Improve Disease Phenotype

The present example demonstrates that, among other things, viral vector compositions comprising a sequence encoding ATP7B (e.g., a truncated ATP7B) may be used to treat or prevent WD (e.g., through reduction of phenotypic effects and/or symptoms associated with disease) in vivo (e.g., in one or more mouse models).

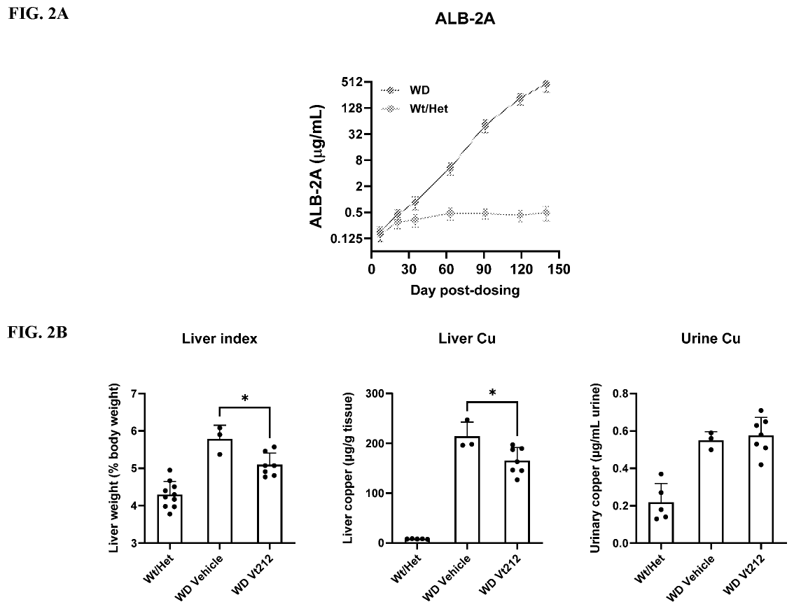

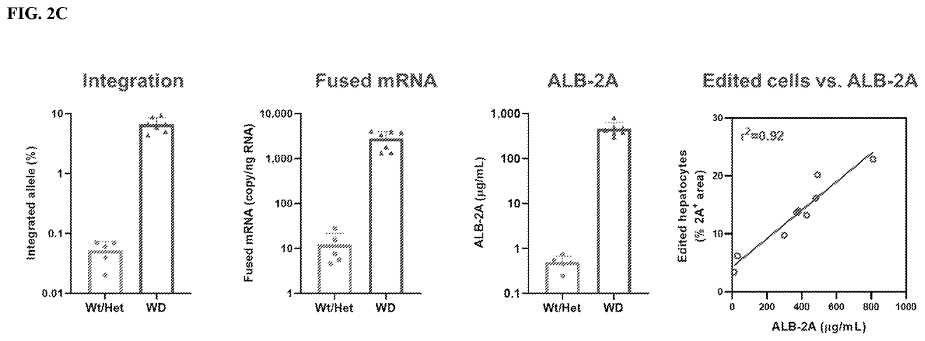

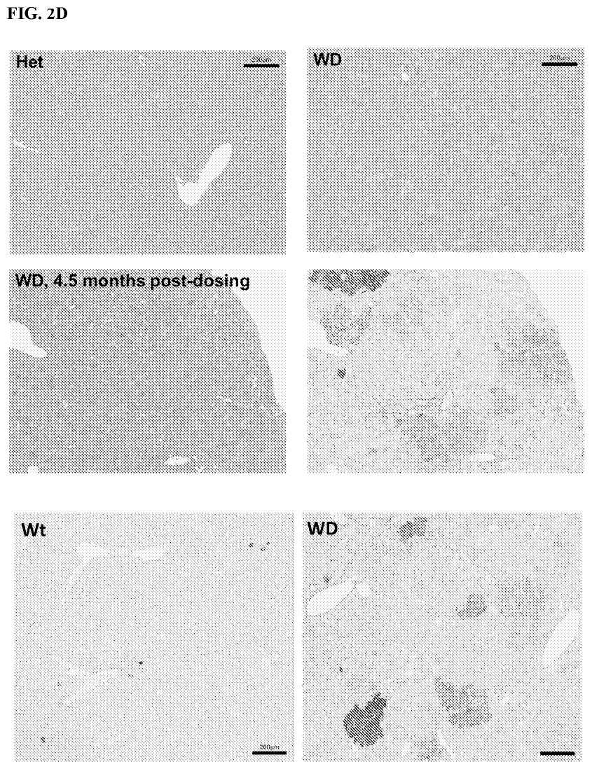

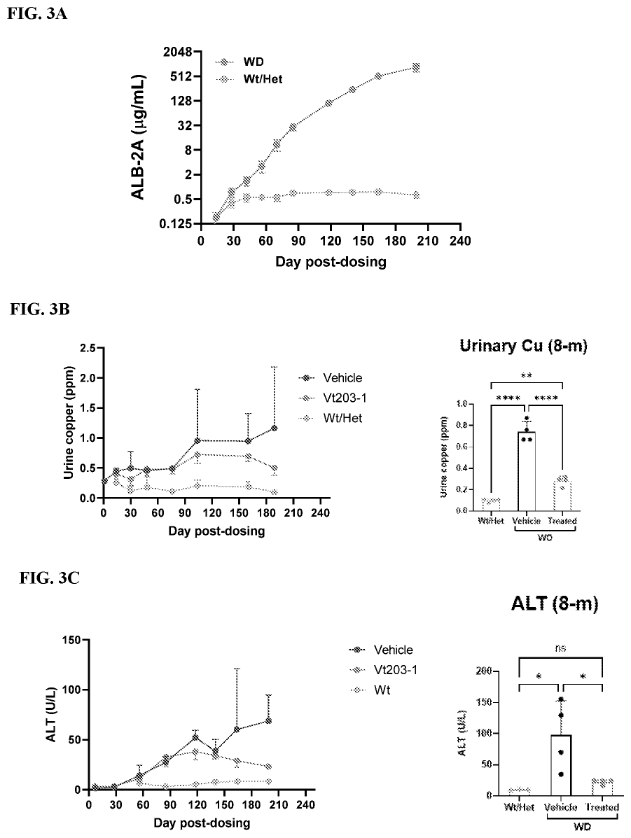

Viral vectors comprising an AAV-DJ viral capsid, human truncated ATP7B transgene, P2A sequence, and flanking homology arms were constructed. Homology arms comprised sequences of even (0.6/0.6 kb, FIG. 2A-2D) or uneven (1.0/0.6 Kb, FIG. 3A-3C) length. Viral vector compositions were intravenously administered at a 1×10¹⁴ vg/kg dose to homozygous WD mice, heterozygous mice, and wild-type mice at post-natal day 21 (P21, FIG. 2A-2D) or P30 (FIG. 3A-3C). Mice were assessed for levels of ALB-2A biomarker (FIG. 2A, FIG. 2C, and FIG. 3A). Mouse liver tissue was harvested and liver weight and copper levels were measured (FIG. 2B) as well as levels of ALT (a marker of liver function, FIG. 3C). Mouse urine was also collected and copper levels were measured (FIG. 2B and FIG. 3B). Harvested liver tissue was also assessed for ATP7B integration levels and ATP7B fused mRNA levels at 4.5 months post-dosing (FIG. 2C). Percentage of edited cells (FIG. 2C) was also estimated through immunohistochemistry analysis, which demonstrated correlation with levels of ALB-2A (FIG. 2C). Liver morphology was also assessed at 4.5 months post-dosing for phenotypic characteristics associated with WD (e.g., enlarged hepatocytes and nucleus, tissue disorganization) in vehicle- and vector-treated mice (FIG. 2D).

Among other things, the present disclosure demonstrates that treatment with viral vectors comprising a sequence encoding a functional ATP7B protein (e.g., truncated ATP7B) may treat or reduce symptoms associated with WD (e.g., reduced liver function, diseased liver phenotypic characteristics, elevated copper levels (e.g., liver or urinary copper levels), elevated blood ALT levels, reduced survival) as compared to a reference (e.g., vehicle treated or untreated).

### Example 4. Viral Vector Compositions can Provide Editing in Humanized Mouse Models

The present example demonstrates that, among other things, viral vector compositions comprising a sequence encoding ATP7B (e.g., truncated ATP7B) administered to a humanized mouse model (e.g., PXB mice) may provide detectable levels of gene integration.

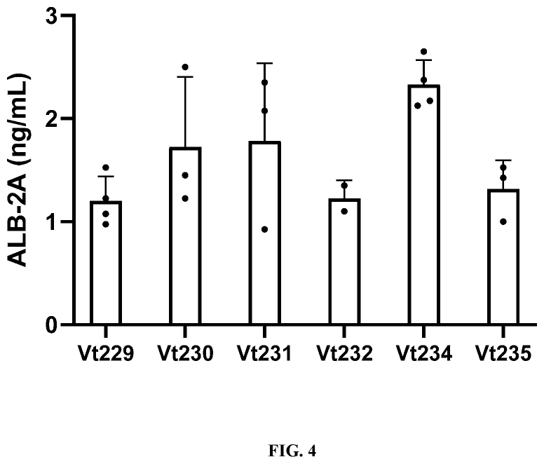

Viral vectors comprising an AAV-sL65 or LK-03 viral capsid, human truncated ATP7B transgene, P2A sequence, and flanking homology arms were constructed as indicated in Table 2 (FIG. 4). Viral vector compositions were intravenously administered at 1×10¹ vg/kg dose to PXB mice at 4 months of age. Mice were assessed for levels of ALB-2A biomarker (FIG. 4).

Among other things, the present disclosure demonstrates that treatment with viral vectors comprising a sequence encoding a functional ATP7B protein (e.g., truncated ATP7B) may provide successful gene integration in a humanized mouse model (e.g., PXB mice). In some embodiments, gene integration in humanized mouse model (e.g., PXB mice) may be specific to humanized cells (e.g., human livers cells) within the model.

### Example 5. Optimization of GeneRide™ Combination Therapies

The present example demonstrates that, among other things, viral vector compositions comprising a sequence encoding ATP7B (e.g., truncated ATP7B) administered to a subject (e.g., a subject suffering from WD) in combination with certain dosages of one or more alternative therapies (e.g., DPA and/or trientine treatment) may optimize selective advantage for cells that have successfully integrated an ATP7B-encoding sequence.

Viral vectors comprising an AAV-DJ viral capsid, human ATP7B transgene, P2A sequence, and flanking homology arms are constructed. Viral vectors herein described are administered at an optimized dose. Mice in all groups are maintained on standard of care (SoC) for a period of time, followed by a titrated dose of SoC. One group of mice is kept on a standard dose of SoC for the duration of the experiment. Mice are then assessed for circulating biomarkers (e.g., levels of ALB-2A).

Among other things, the present disclosure demonstrates that treatment of a subject (e.g., a subject suffering from WD) with viral vectors of the present disclosure may comprise administration of viral vectors in combination with one or more alternative therapies (e.g. alternative WD therapy). In some embodiments, dosage level of one or more alternative therapies (e.g., WD's SoC therapies) may be titrated to provide a selective advantage for cells (e.g., liver cells) while controlling disease severity (e.g., reducing symptoms and/or side effects of disease)

### Example 6. GENERIDE™ Treatment Allows Rapid Selective Expansion of Edited Cells In Vivo

The present example further confirms that viral vector compositions comprising a sequence encoding ATP7B (e.g., a truncated ATP7B) may be used to treat or prevent WD (e.g., through reduction of one or more phenotypic effects and/or symptoms associated with disease) in vivo (e.g., in one or more mouse models) and provide a selective advantage for cells that have successfully integrated a ATP7B-encoding sequence.

Viral vectors comprising an AAV-DJ viral capsid, human truncated ATP7B transgene, P2A sequence, and flanking homology arms were constructed. 3-week-old WD, wild-type, or heterozygous (WT/Het) healthy control mice were injected intravenously with a 1×10¹⁴ vg/kg dose of viral vectors described herein or with vehicle (n=5-7 per group). Tissues from treated mice were harvested at 25 weeks of age and analyzed for genomic DNA (as described in Example 1) and fused mRNA (as described in Example 1).

In addition, harvested tissues were analyzed for a presence of ATP7B. Briefly, slides were incubated with primary antibody (anti-human ATP7B antibody [ABCAM cat #ab124973; 1:100]) for 1 hour at room temperature. Followed by incubation of Ultra Streptavidin HRP Kit according to manufacturer's instruction (BioLegend cat #929501). Slides were imaged using a digital slide scanner (Hamamatsu, Bridgewater, NJ) or a compound microscope (AmScope cat #B100B-5M).

Further, harvested tissues were analyzed for copper staining. Briefly, slides were deparaffinized with xylene and rehydrated with ethanol and water. Slides were then incubated with 0.5% ammonium sulfide (VWR) for 5 min at room temperature, rinsed with water and incubated with 0.1N HCl for 3 min. Slides were incubated with the developer solution for 10 min. The developer solution was made of one part 5% silver nitrate (VWR) and five parts of a solution consisting of 2% w/v hydroquinone (Fisher Scientific) and 5% w/v citric acid (Fisher Scientific).

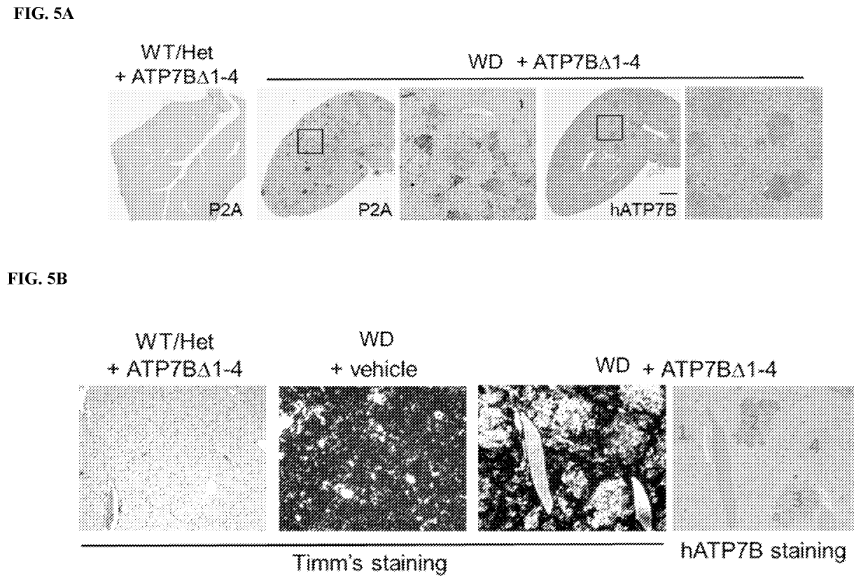

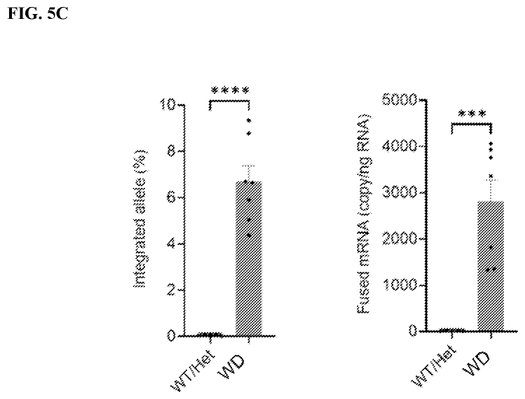

As demonstrated in FIGS. 5A and 5B, liver tissue from WD mice administered GENERIDE™ treatment, exhibited robust staining for hepatocytes expressing fused P2A tag and human ATP7B. Timm's staining and ATP7B histochemical staining conducted in consecutive liver slices showed extensive and homogeneous copper accumulation in untreated mice while in contrast, while, slices from GENERIDE™ treated mice showed clustered cells expressing human ATP7B without the presence of copper staining. Moreover, as demonstrated in FIG. 5C, viral vector described herein effectively integrated into the targeting site (as exhibited by a mean integrated allele % of at least about 6% and mean fused mRNA of at least about 2500 copy/ng RNA).

Thus, the present example demonstrates that viral vector compositions comprising a sequence encoding ATP7B (e.g., a truncated ATP7B) may provide a selective advantage for cells that have successfully integrated a ATP7B-encoding sequence characterized by robust staining for hepatocytes expressing fused P2A tag and human ATP7B without the presence of copper staining, and a mean integrated allele % of at least about 6% and mean fused mRNA of at least about 2500 copy/ng RNA.

### Example 7. GENERIDE™ Treatment Improves Liver Disease in WD Mice

The present example further confirms that, viral vector compositions comprising a sequence encoding ATP7B (e.g., a truncated ATP7B) may be used to treat or prevent WD (e.g., through reduction of phenotypic effects and/or symptoms associated with disease) in vivo (e.g., in one or more mouse models).

Viral vectors comprising an AAV-DJ viral capsid, human truncated ATP7B transgene, P2A sequence, and flanking homology arms were constructed. 4-week-old WD, wild-type, or heterozygous (WT/Het) healthy control mice were injected intravenously with a 1×10¹⁴ vg/kg dose of viral vectors described herein or with vehicle (n=4 per group). Tissues from treated mice were harvested at 36 weeks of age and analyzed for cellular and tissue structure (through Hematoxylin and Eosin (H&E) staining), ALT levels (as described in Example 1), and liver and urinary copper levels (as described in Example 1).

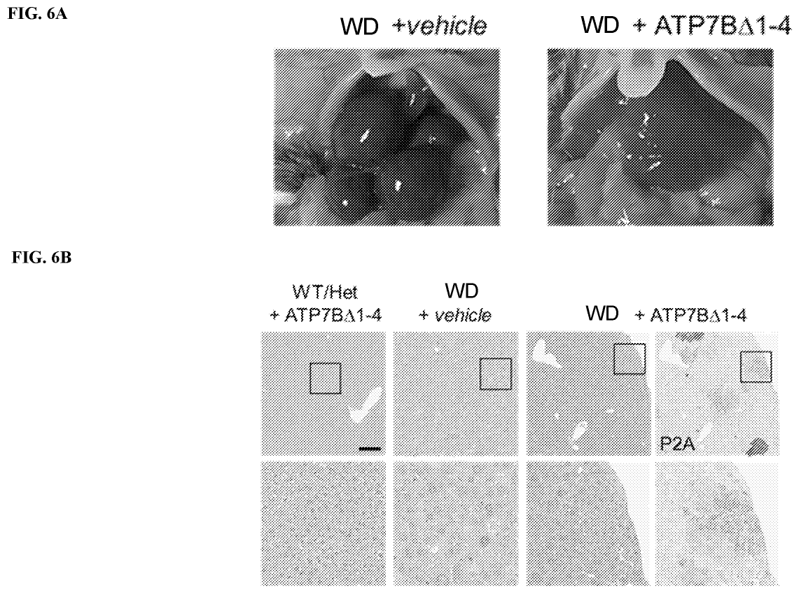

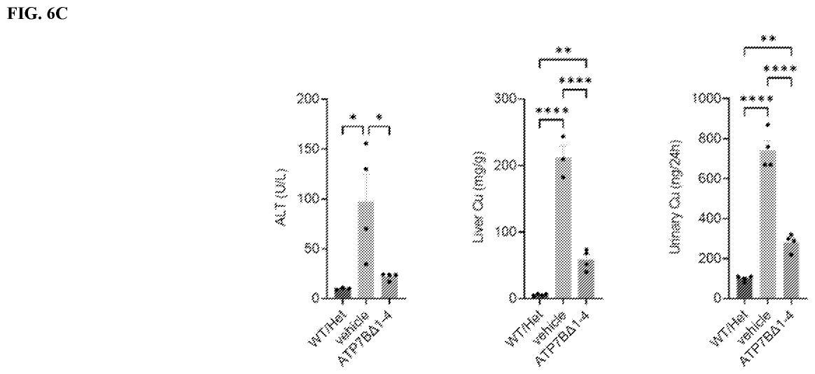

As demonstrated in FIG. 6A, livers from WD mice treated with vehicle exhibited fibrotic nodules. In contrast, livers from WD mice administered GENERIDE™ treatment exhibited normal morphology and cell size (e.g., cell morphology similar to wild-type or heterozygous (WT/Het) healthy control mice). Furthermore, as demonstrated in FIG. 6B, treatment with viral vector composition described herein significantly improved liver damage with repopulated areas expressing ATP7B and exhibiting normal cell morphology (e.g., cell morphology similar to wild-type or heterozygous (WT/Het) healthy control mice).

In addition, the present example demonstrates that GENERIDE™ treatment administered to WD mice may significantly improve liver function (as exhibited by a reduction in ALT levels and reduction in liver and urinary copper levels). As demonstrated in FIG. 6C, mean ALT levels were at least about 25 U/L for mice administered GENERIDE™ treatment, while mice administered vehicle had a mean ALT level of at least about 90 U/L. There was more variability in ALT levels for mice administered vehicle (as exhibited by an ALT level of at least 30-125 U/L) as compared to mice administered GENERIDE™ treatment (as exhibited by an ALT level of at least about 25 U/L). Liver and urinary samples from mice administered vehicle had a mean copper measurements of at least about 200 mg/g and 750 ng/24h, respectfully, while, liver and urinary samples from mice administered GENERIDE™ treatment had a mean copper measurements of at least about 50 mg/g and 210 ng/24h, respectfully.

Thus, the present example demonstrates that viral vector compositions comprising a sequence encoding ATP7B (e.g., a truncated ATP7B) may be used to treat or prevent WD (e.g., through reduction of phenotypic effects and/or symptoms associated with disease) in vivo (e.g., in one or more mouse models).

### Example 8. Assessment of Canonical Gene Therapy Construct In-Vivo

The present demonstrates that canonical AAV viral vector encoding a human ATB7B may be used to treat or prevent WD (e.g., through reduction of phenotypic effects and/or symptoms associated with disease) in vivo (e.g., in one or more mouse models).

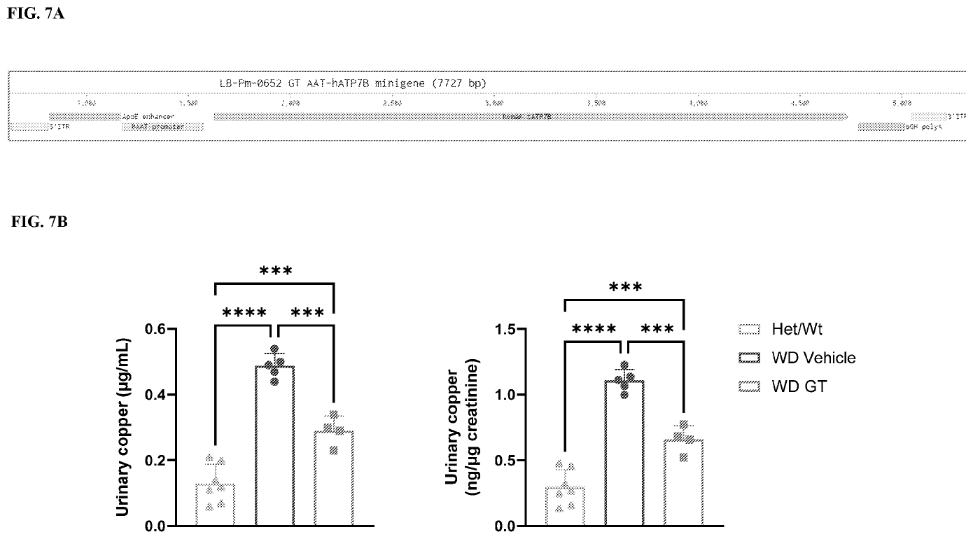

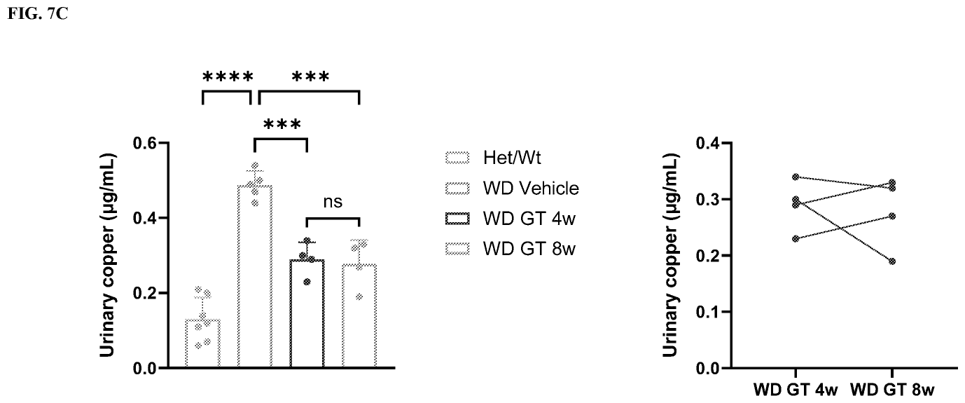

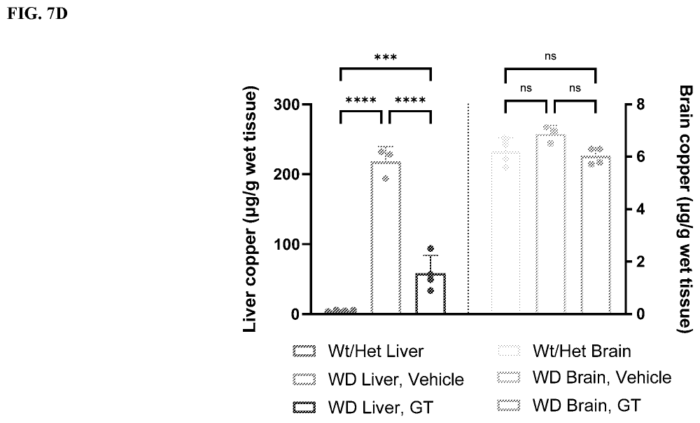

An AAV construct, as described in Example 8 (FIG. 7A), was generated. WD mice were intravenously dosed with AAV8-hATP7B vector (at a dose of 1E13 vg/kg) or formulation buffer at 10 weeks of age. Urine and blood samples were collected once a month. Animals were harvested 2 months after dosing. Urine and liver copper levels were assessed.

As demonstrated in FIG. 7B, urinary copper levels were reduced in mice at least 4 weeks post-administration of AAV8-hATP7B vector (exhibited by a mean urinary copper level of at least about 0.25 μg/mL and 0.6 ng/μg creatinine) as compared to mice at least 4 weeks post-administration of vehicle (exhibited by a mean urinary copper level of at least about 0.45 μg/mL and 1.1 ng/μg creatinine). Mean urinary copper levels for Het/WT mice were at least about 0.15 μg/mL and 0.25 ng/μg creatinine. Further, as demonstrated in FIG. 7C, urinary copper levels remained low in WD mice at least 8 weeks post-dosing (as exhibited by a mean copper level of at least about 0.25 μg/mL). Samples from individual WD mouse administered an AAV8-hATP7B vector exhibited a copper level of at least about 0.20 to 0.35 μg/mL In addition, as demonstrated in FIG. 7D, there was not a significant increase in brain copper level 4 month post-dosing.

Thus, the present example demonstrates that canonical AAV viral vector encoding a human ATB7B may be used to treat or prevent WD (e.g., through reduction of phenotypic effects and/or symptoms associated with disease) in vivo (e.g., in one or more mouse models) characterized by reduction in urinary copper levels

### Example 9: Two-Plasmid and Three-Plasmid Systems May be Used to Produce Viral Vectors

The present example demonstrates that, among other things, a two-plasmid or three-plasmid system may be used to produce AAV vectors.

In some embodiments, HEK293F cells are expanded for use in vector production. Cells are split to 2e6 cells/mL in 200 mL of Expi293 media in a 500 mL flask. Plasmid mixes for various transfection conditions are made and filtered through a 0.22 μM filter unit. A transfection reagent mix (e.g., PEI or FectoVIR-AAV) is prepared according to manufacturer's protocol. Plasmid and transfection reagent mixes are combined to produce a single transfection mix. 20 mL of transfection mix is added to 100 mL of HEK293F cells in a 500 mL flask and allowed to incubate at 37° C. for 72 hours.

In some embodiments, plasmids used in a two-plasmid system comprise an AAV rep sequence and relevant sequences from a helper viruses (“Rep/Helper Plasmid”) or an AAV cap sequence and a payload (“Payload/Cap Plasmid”). In some embodiments, plasmids used in a three-plasmid system comprise separate plasmids, each encoding one of: 1) an AAV rep and AAV cap sequence, 2) relevant sequence from a helper virus, and 3) a payload. A human gene of interest sequence with flanking homology arms for mouse albumin (e.g., “mHA-ATP7B”), which is compatible with a GeneRide system, may be used as the payload for experiments in mice. A human gene of interest sequence with flanking homology arms for human albumin (“hHA-ATP7B”), which is compatible with a GeneRide system, may be used as the payload for experiments in humans or humanized mice. In some embodiments, a payload may comprise SEQ ID NO: 41. In some embodiments, a payload may consist of SEQ ID NO: 41. In some embodiments, a payload may comprise any payload described herein. A variety of AAV cap genes encoding different AAV capsids are assessed within the Payload/Cap plasmid. In some embodiments, the AAV cap gene may encode a AAV1, AAV2, AAV3, AAV4, AAV5, AAV6, AAV7, AAV8, AAV9, AAV10, AAV11, AAVC11.01, AAVC11.02, AAVC11.03, AAVC11.04, AAVC11.05, AAVC11.06, AAVC11.07, AAVC11.08, AAVC11.09, AAVC11.10, AAVC11.11, AAVC11.12, AAVC11.13, AAVC11.14, AAVC11.15, AAVC11.16, AAVC11.17, AAVC11.18, AAVC11.19, AAV-DJ, AAV-LK03, AAV-LK19, AAVrh.74, AAVrh.10, AAVhu.37, AAVrh.K, AAVrh.39, AAV12, AAV 13, AAVrh.8, avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype). In some embodiments, a Payload/Cap plasmid may comprise SEQ ID NO: 42. In some embodiments, a Payload/Cap plasmid may consist of SEQ ID NO: 42. In some embodiments, a Payload/Cap plasmid may comprise SEQ ID NO: 43. In some embodiments, a Payload/Cap plasmid may consist of SEQ ID NO: 43. In some embodiments, a Payload/Cap plasmid may comprise any payload or capsid sequence disclosed herein.

TABLE 1B
Exemplary sequences for production of viral vectors.

| | | SEQ ID |
|---|---|---|
| Name | Sequence (5′ to 3′) | NO |
| Gene of | gcatgtttggttaggctagggcttagggatttatatatcaaaggaggctttgtacatgtgg | 41 |
| interest | gacagggatcttattttacaaacaattgtcttacaaaatgaataaaacagcactttgtttttat | |
| (GOI) | ctcctgctctattgtgccatactgttaaatgtttataatgcctgttctgtttccaaatttgtgatg | |
| cassette | cttatgaatattaataggaatatttgtaaggcctgaaatattttgatcatgaaatcaaaacatt | |
| | aatttatttaaacatttacttgaaatgtggtggtttgtgatttagttgattttataggctagtgg | |
| | gagaatttacattcaaatgtctaaatcacttaaaattgccctttatggcctgacagtaactttt | |
| | ttttattcatttggggacaactatgtccgtgagcttccgtccagagattatagtagtaaattg | |
| | taattaaaggatatgatgcacgtgaaatcactttgcaatcatcaatagcttcataaatgttaa | |
| | ttttgtatcctaatagtaatgctaatattttcctaacatctgtcatgtctttgtgttcagggtaaa | |
| | aaacttgttgctgcaagtcaagctgccttaggcttaggcagcggcgccaccaacttcag | |
| | cctgctgaaacaggccGGCGACGTGGAAGAGAACCCTGGCCC | |
| | TCCTGAGCAGGAGAGACAGATCACAGCCAGAGAAG | |
| | GGGCCAGTCGGAAAATCTTATCTAAGCTTTCTTTGCC | |
| | TACCCGTGCCTGGGAACCAGCAATGAAGAAGAGTTT | |
| | TGCTTTTGACAATGTTGGCTATGAAGGTGGTCTGGAT | |
| | GGCCTGGGCCCTTCTTCTCAGCCGCAGAAGTGCTTCT | |
| | TACAGATCAAAGGCATGACCTGTGCATCCTGTGTGTC | |
| | TAACATAGAAAGGAATCTGCAGAAAGAAGCTGGTGT | |
| | TCTCTCCGTGTTGGTTGCCTTGATGGCAGGAAAGGCA | |
| | GAGATCAAGTATGACCCAGAGGTCATCCAGCCCCTC | |
| | GAGATAGCTCAGTTCATCCAGGACCTGGGTTTTGAG | |
| | GCAGCAGTCATGGAGGACTACGCAGGCTCCGATGGC | |
| | AACATTGAGCTGACAATCACAGGGATGACCTGCGCG | |
| | TCCTGTGTCCACAACATAGAGTCCAAACTCACGAGG | |
| | ACAAATGGCATCACTTATGCCTCCGTTGCCCTTGCCA | |
| | CCAGCAAAGCCCTTGTTAAGTTTGACCCGGAAATTAT | |
| | CGGTCCACGGGATATTATCAAAATTATTGAGGAAAT | |
| | TGGCTTTCATGCTTCCCTGGCCCAGAGAAACCCCAAC | |
| | GCTCATCACTTGGACCACAAGATGGAAATAAAGCAG | |
| | TGGAAGAAGTCTTTCCTGTGCAGCCTGGTGTTTGGCA | |
| | TCCCTGTCATGGCCTTAATGATCTATATGCTGATACC | |
| | CAGCAACGAGCCCCACCAGTCCATGGTCCTGGACCA | |
| | CAACATCATTCCAGGACTGTCCATTCTAAATCTCATC | |
| | TTCTTTATCTTGTGTACCTTTGTCCAGCTCCTCGGTGG | |
| | GTGGTACTTCTACGTTCAGGCCTACAAATCTCTGAGA | |
| | CACAGGTCAGCCAACATGGACGTGCTCATCGTCCTG | |
| | GCCACAAGCATTGCTTATGTTTATTCTCTGGTCATCC | |
| | TGGTGGTTGCTGTGGCTGAGAAGGCGGAGAGGAGCC | |
| | CTGTGACATTCTTCGACACGCCCCCCATGCTCTTTGT | |
| | GTTCATTGCCCTGGGCCGGTGGCTGGAACACTTGGCA | |
| | AAGAGCAAAACCTCAGAAGCCCTGGCTAAACTCATG | |
| | TCTCTCCAAGCCACAGAAGCCACCGTTGTGACCCTTG | |
| | GTGAGGACAATTTAATCATCAGGGAGGAGCAAGTCC | |
| | CCATGGAGCTGGTGCAGCGGGGCGATATCGTCAAGG | |
| | TGGTCCCTGGGGGAAAGTTTCCAGTGGATGGGAAAG | |
| | TCCTGGAAGGCAATACCATGGCTGATGAGTCCCTCAT | |
| | CACAGGAGAAGCCATGCCAGTCACTAAGAAACCCGG | |
| | AAGCACTGTAATTGCGGGGTCTATAAATGCACATGG | |
| | CTCTGTGCTCATTAAAGCTACCCACGTGGGCAATGAC | |
| | ACCACTTTGGCTCAGATTGTGAAACTGGTGGAAGAG | |
| | GCTCAGATGTCAAAGGCACCCATTCAGCAGCTGGCT | |
| | GACCGGTTTAGTGGATATTTTGTCCCATTTATCATCA | |
| | TCATGTCAACTTTGACGTTGGTGGTATGGATTGTAAT | |
| | CGGTTTTATCGATTTTGGTGTTGTTCAGAGATACTTTC | |
| | CTAACCCCAACAAGCACATCTCCCAGACAGAGGTGA | |
| | TCATCCGGTTTGCTTTCCAGACGTCCATCACGGTGCT | |
| | GTGCATTGCCTGCCCCTGCTCCCTGGGGCTGGCCACG | |
| | CCCACGGCTGTCATGGTGGGCACCGGGGTGGCCGCG | |
| | CAGAACGGCATCCTCATCAAGGGAGGCAAGCCCCTG | |
| | GAGATGGCGCACAAGATAAAGACTGTGATGTTTGAC | |
| | AAGACTGGCACCATTACCCATGGCGTCCCCAGGGTC | |
| | ATGCGGGTGCTCCTGCTGGGGGATGTGGCCACACTG | |
| | CCCCTCAGGAAGGTTCTGGCTGTGGTGGGGACTGCG | |
| | GAGGCCAGCAGTGAACACCCCTTGGGCGTGGCAGTC | |
| | ACCAAATACTGTAAAGAGGAACTTGGAACAGAGACC | |
| | TTGGGATACTGCACGGACTTCCAGGCAGTGCCAGGC | |
| | TGTGGAATTGGGTGCAAAGTCAGCAACGTGGAAGGC | |
| | ATCCTGGCCCACAGTGAGCGCCCTTTGAGTGCACCG | |
| | GCCAGTCACCTGAATGAGGCTGGCAGCCTTCCCGCA | |
| | GAAAAAGATGCAGTCCCCCAGACCTTCTCTGTGCTG | |
| | ATTGGAAACCGTGAGTGGCTGAGGCGCAACGGTTTA | |
| | ACCATTTCTAGCGATGTCAGTGACGCTATGACAGACC | |
| | ACGAGATGAAAGGACAGACAGCCATCCTGGTGGCTA | |
| | TTGACGGTGTGCTCTGTGGGATGATCGCAATCGCAG | |
| | ACGCTGTCAAGCAGGAGGCTGCCCTGGCTGTGCACA | |
| | CGCTGCAGAGCATGGGTGTGGACGTGGTTCTGATCA | |
| | CGGGGGACAACCGGAAGACAGCCAGAGCTATTGCCA | |
| | CCCAGGTTGGCATCAACAAAGTCTTTGCAGAGGTGC | |
| | TGCCTTCGCACAAGGTGGCCAAGGTCCAGGAGCTCC | |
| | AGAATAAAGGGAAGAAAGTCGCCATGGTGGGGGAT | |
| | GGGGTCAATGACTCCCCGGCCTTGGCCCAGGCAGAC | |
| | ATGGGTGTGGCCATTGGCACCGGCACGGATGTGGCC | |
| | ATCGAGGCAGCCGACGTCGTCCTTATCAGAAATGAT | |
| | TTGCTGGATGTGGTGGCTAGCATTCACCTTTCCAAGA | |
| | GGACTGTCCGAAGGATACGCATCAACCTGGTCCTGG | |
| | CACTGATTTATAACCTGGTTGGGATACCCATTGCAGC | |
| | AGGTGTCTTCATGCCCATCGGCATTGTGCTGCAGCCC | |
| | TGGATGGGCTCAGCGGCCATGGCAGCCTCCTCTGTGT | |
| | CTGTGGTGCTCTCATCCCTGCAGCTCAAGTGCTATAA | |
| | GAAGCCTGACCTGGAGAGGTATGAGGCACAGGCGCA | |
| | TGGCCACATGAAGCCCCTGACGGCATCCCAGGTCAG | |
| | TGTGCACATAGGCATGGATGACAGGTGGCGGGACTC | |
| | CCCCAGGGCCACACCATGGGACCAGGTCAGCTATGT | |
| | CAGCCAGGTGTCGCTGTCCTCCCTGACGTCCGACAAG | |
| | CCATCTCGGCACAGCGCTGCAGCAGACGATGATGGG | |
| | GACAAGTGGTCTCTGCTCCTGAATGGCAGGGATGAG | |
| | GAGCAGTACATCtaacatcacatttaaaagcatctcaggtaactatattttgaat | |
| | tttttaaaaaagtaactataatagttattattaaaatagcaaagattgaccatttccaagagc | |
| | catatagaccagcaccgaccactattctaaactatttatgtatgtaaatattagcttttaaaat | |
| | tctcaaaatagttgctgagttgggaaccactattatttctattttgtagatgagaaaatgaag | |
| | ataaacatcaaagcatagattaagtaattttccaaagggtcaaaattcaaaattgaaacca | |
| | aagtttcagtgttgcccattgtcctgttctgacttatatgatgcggtacacagagccatcca | |
| | agtaagtgatggctcagcagtggaatactctgggaattaggctgaaccacatgaaaga | |
| | gtgctttatagggcaaaaacagttgaatatcagtgatttcacatggttcaacctaatagttc | |
| | aactcatcctttccattggagaatatgatggatctaccttctgtgaactttatagtgaagaat | |
| | ctgctattacatttccaatttgtcaacatgctgagctttaataggacttatcttcttatgacaac | |
| | atttattg | |
| LK03-GR | atggctgctgacggttatcttccagattggctcgaggacaacctttctgaaggcattcga | 42 |
| hATP7B | gagtggtgggcgctgcaacctggagcccctaaacccaaggcaaatcaacaacatcag | |
| | gacaacgctcggggtcttgtgcttccgggttacaaatacctcggacccggcaacggact | |
| | cgacaagggggaacccgtcaacgcagcggacgcggcagccctcgagcacgacaag | |
| | gcctacgaccagcagctcaaggccggtgacaacccctacctcaagtacaaccacgcc | |
| | gacgccgagttccaggagcggctcaaagaagatacgtcttttgggggcaacctcggg | |
| | cgagcagtcttccaggccaaaaagaggcttcttgaacctcttggtctggttgaggaagc | |
| | ggctaagacggctcctggaaagaagaggcctgtagatcagtctcctcaggaaccgga | |
| | ctcatcatctggtgttggcaaatcgggcaaacagcctgccagaaaaagactaaatttcg | |
| | gtcagactggcgactcagagtcagtcccagaccctcaacctctcggagaaccaccagc | |
| | agcccccacaagtttgggatctaatacaatggcttcaggcggtggcgcaccaatggca | |
| | gacaataacgagggtgccgatggagtgggtaattcctcaggaaattggcattgcgattc | |
| | ccaatggctgggcgacagagtcatcaccaccagcaccagaacctgggccctgcccac | |
| | ttacaacaaccatctctacaagcaaatctccagccaatcaggagcttcaaacgacaacc | |
| | actactttggctacagcaccccttgggggtattttgactttaacagattccactgccacttct | |
| | caccacgtgactggcagcgactcattaacaacaactggggattccggcccaagaaact | |
| | cagcttcaagctcttcaacatccaagttaaagaggtcacgcagaacgatggcacgacg | |
| | actattgccaataaccttaccagcacggttcaagtgtttacggactcggagtatcagctcc | |
| | cgtacgtgctcgggtcggcgcaccaaggctgtctcccgccgtttccagcggacgtcttc | |
| | atggtccctcagtatggatacctcaccctgaacaacggaagtcaagcggtgggacgct | |
| | catccttttactgcctggagtacttcccttcgcagatgctaaggactggaaataacttccaa | |
| | ttcagctataccttcgaggatgtaccttttcacagcagctacgctcacagccagagtttgg | |
| | atcgcttgatgaatcctcttattgatcagtatctgtactacctgaacagaacgcaaggaac | |
| | aacctctggaacaaccaaccaatcacggctgctttttagccaggctgggcctcagtctat | |
| | gtctttgcaggccagaaattggctacctgggccctgctaccggcaacagagactttcaa | |
| | agactgctaacgacaacaacaacagtaactttccttggacagcggccagcaaatatcat | |
| | ctcaatggccgcgactcgctggtgaatccaggaccagctatggccagtcacaaggac | |
| | gatgaagaaaaatttttccctatgcacggcaatctaatatttggcaaagaagggacaacg | |
| | gcaagtaacgcagaattagataatgtaatgattacggatgaagaagagattcgtaccac | |
| | caatcctgtggcaacagagcagtatggaactgtggcaaataacttgcagagctcaaata | |
| | cagctcccacgactagaactgtcaatgatcagggggccttacctggcatggtgtggcaa | |
| | gatcgtgacgtgtaccttcaaggacctatctgggcaaagattcctcacacggatggaca | |
| | ctttcatccttctcctctgatgggaggctttggactgaaacatccgcctcctcaaatcatga | |
| | tcaaaaatactccggtaccggcaaatcctccgacgactttcagcccggccaagtttgctt | |
| | catttatcactcagtactccactggacaggtcagcgtggaaattgagtgggagctacag | |
| | aaagaaaacagcaaacgttggaatccagagattcagtacacttccaactacaacaagtc | |
| | tgttaatgtggactttactgtagacactaatggtgtttatagtgaacctcgccccattggca | |
| | cccgttaccttacccgtcccctgtaattgcttgttaatcaataaaccgtttaattcgtttcagtt | |
| | gaactttggtctctgcgtatttctttcttatctagtttccatatgcatgtagataagtagcatgg | |
| | cgggttaatcattaactaaccggtacctctagaactatagctagcgatgaccctgctgatt | |
| | ggttcgctgaccatttccgggtgcgggacggcgttaccagaaactcagaaggttcgtcc | |
| | aaccaaaccgactctgacggcagtttacgagagagatgatagggtctgcttcagtaagc | |
| | cagatgctacacaattaggcttgtacatattgtcgttagaacgcggctacaattaatacata | |
| | accttatgtatcatacacatacgatttaggtgacactatagaatacacggaattaattcttg | |
| | gccactccctctctgcgcgctcgctcgctcactgaggccgcccgggcaaagcccggg | |
| | cgtcgggcgacctttggtcgcccggcctcagtgagcgagcgagcgcgcagagaggg | |
| | agtggccaactccatcactaggggttcctgcatgtttggttaggctagggcttagggattt | |
| | atatatcaaaggaggctttgtacatgtgggacagggatcttattttacaaacaattgtctta | |
| | caaaatgaataaaacagcactttgtttttatctcctgctctattgtgccatactgttaaatgttt | |
| | ataatgcctgttctgtttccaaatttgtgatgcttatgaatattaataggaatatttgtaaggc | |
| | ctgaaatattttgatcatgaaatcaaaacattaatttatttaaacatttacttgaaatgtggtg | |
| | gtttgtgatttagttgattttataggctagtgggagaatttacattcaaatgtctaaatcactta | |
| | aaattgccctttatggcctgacagtaacttttttttattcatttggggacaactatgtccgtga | |
| | gcttccgtccagagattatagtagtaaattgtaattaaaggatatgatgcacgtgaaatca | |
| | ctttgcaatcatcaatagcttcataaatgttaattttgtatcctaatagtaatgctaatattttcc | |
| | taacatctgtcatgtctttgtgttcagggtaaaaaacttgttgctgcaagtcaagctgcctta | |
| | ggcttaggcagcggcgccaccaacttcagcctgctgaaacaggccGGCGACG | |
| | TGGAAGAGAACCCTGGCCCTCCTGAGCAGGAGAGAC | |
| | AGATCACAGCCAGAGAAGGGGCCAGTCGGAAAATCT | |
| | TATCTAAGCTTTCTTTGCCTACCCGTGCCTGGGAACC | |
| | AGCAATGAAGAAGAGTTTTGCTTTTGACAATGTTGGC | |
| | TATGAAGGTGGTCTGGATGGCCTGGGCCCTTCTTCTC | |
| | AGCCGCAGAAGTGCTTCTTACAGATCAAAGGCATGA | |
| | CCTGTGCATCCTGTGTGTCTAACATAGAAAGGAATCT | |
| | GCAGAAAGAAGCTGGTGTTCTCTCCGTGTTGGTTGCC | |
| | TTGATGGCAGGAAAGGCAGAGATCAAGTATGACCCA | |
| | GAGGTCATCCAGCCCCTCGAGATAGCTCAGTTCATCC | |
| | AGGACCTGGGTTTTGAGGCAGCAGTCATGGAGGACT | |
| | ACGCAGGCTCCGATGGCAACATTGAGCTGACAATCA | |
| | CAGGGATGACCTGCGCGTCCTGTGTCCACAACATAG | |
| | AGTCCAAACTCACGAGGACAAATGGCATCACTTATG | |
| | CCTCCGTTGCCCTTGCCACCAGCAAAGCCCTTGTTAA | |
| | GTTTGACCCGGAAATTATCGGTCCACGGGATATTATC | |
| | AAAATTATTGAGGAAATTGGCTTTCATGCTTCCCTGG | |
| | CCCAGAGAAACCCCAACGCTCATCACTTGGACCACA | |
| | AGATGGAAATAAAGCAGTGGAAGAAGTCTTTCCTGT | |
| | GCAGCCTGGTGTTTGGCATCCCTGTCATGGCCTTAAT | |
| | GATCTATATGCTGATACCCAGCAACGAGCCCCACCA | |
| | GTCCATGGTCCTGGACCACAACATCATTCCAGGACTG | |
| | TCCATTCTAAATCTCATCTTCTTTATCTTGTGTACCTT | |
| | TGTCCAGCTCCTCGGTGGGTGGTACTTCTACGTTCAG | |
| | GCCTACAAATCTCTGAGACACAGGTCAGCCAACATG | |
| | GACGTGCTCATCGTCCTGGCCACAAGCATTGCTTATG | |
| | TTTATTCTCTGGTCATCCTGGTGGTTGCTGTGGCTGA | |
| | GAAGGCGGAGAGGAGCCCTGTGACATTCTTCGACAC | |
| | GCCCCCCATGCTCTTTGTGTTCATTGCCCTGGGCCGG | |
| | TGGCTGGAACACTTGGCAAAGAGCAAAACCTCAGAA | |
| | GCCCTGGCTAAACTCATGTCTCTCCAAGCCACAGAA | |
| | GCCACCGTTGTGACCCTTGGTGAGGACAATTTAATCA | |
| | TCAGGGAGGAGCAAGTCCCCATGGAGCTGGTGCAGC | |
| | GGGGCGATATCGTCAAGGTGGTCCCTGGGGGAAAGT | |
| | TTCCAGTGGATGGGAAAGTCCTGGAAGGCAATACCA | |
| | TGGCTGATGAGTCCCTCATCACAGGAGAAGCCATGC | |
| | CAGTCACTAAGAAACCCGGAAGCACTGTAATTGCGG | |
| | GGTCTATAAATGCACATGGCTCTGTGCTCATTAAAGC | |
| | TACCCACGTGGGCAATGACACCACTTTGGCTCAGATT | |
| | GTGAAACTGGTGGAAGAGGCTCAGATGTCAAAGGCA | |
| | CCCATTCAGCAGCTGGCTGACCGGTTTAGTGGATATT | |
| | TTGTCCCATTTATCATCATCATGTCAACTTTGACGTTG | |
| | GTGGTATGGATTGTAATCGGTTTTATCGATTTTGGTG | |
| | TTGTTCAGAGATACTTTCCTAACCCCAACAAGCACAT | |
| | CTCCCAGACAGAGGTGATCATCCGGTTTGCTTTCCAG | |
| | ACGTCCATCACGGTGCTGTGCATTGCCTGCCCCTGCT | |
| | CCCTGGGGCTGGCCACGCCCACGGCTGTCATGGTGG | |
| | GCACCGGGGTGGCCGCGCAGAACGGCATCCTCATCA | |
| | AGGGAGGCAAGCCCCTGGAGATGGCGCACAAGATA | |
| | AAGACTGTGATGTTTGACAAGACTGGCACCATTACC | |
| | CATGGCGTCCCCAGGGTCATGCGGGTGCTCCTGCTGG | |
| | GGGATGTGGCCACACTGCCCCTCAGGAAGGTTCTGG | |
| | CTGTGGTGGGGACTGCGGAGGCCAGCAGTGAACACC | |
| | CCTTGGGCGTGGCAGTCACCAAATACTGTAAAGAGG | |
| | AACTTGGAACAGAGACCTTGGGATACTGCACGGACT | |
| | TCCAGGCAGTGCCAGGCTGTGGAATTGGGTGCAAAG | |
| | TCAGCAACGTGGAAGGCATCCTGGCCCACAGTGAGC | |
| | GCCCTTTGAGTGCACCGGCCAGTCACCTGAATGAGG | |
| | CTGGCAGCCTTCCCGCAGAAAAAGATGCAGTCCCCC | |
| | AGACCTTCTCTGTGCTGATTGGAAACCGTGAGTGGCT | |
| | GAGGCGCAACGGTTTAACCATTTCTAGCGATGTCAGT | |
| | GACGCTATGACAGACCACGAGATGAAAGGACAGAC | |
| | AGCCATCCTGGTGGCTATTGACGGTGTGCTCTGTGGG | |
| | ATGATCGCAATCGCAGACGCTGTCAAGCAGGAGGCT | |
| | GCCCTGGCTGTGCACACGCTGCAGAGCATGGGTGTG | |
| | GACGTGGTTCTGATCACGGGGGACAACCGGAAGACA | |
| | GCCAGAGCTATTGCCACCCAGGTTGGCATCAACAAA | |
| | GTCTTTGCAGAGGTGCTGCCTTCGCACAAGGTGGCCA | |
| | AGGTCCAGGAGCTCCAGAATAAAGGGAAGAAAGTC | |
| | GCCATGGTGGGGGATGGGGTCAATGACTCCCCGGCC | |
| | TTGGCCCAGGCAGACATGGGTGTGGCCATTGGCACC | |
| | GGCACGGATGTGGCCATCGAGGCAGCCGACGTCGTC | |
| | CTTATCAGAAATGATTTGCTGGATGTGGTGGCTAGCA | |
| | TTCACCTTTCCAAGAGGACTGTCCGAAGGATACGCAT | |
| | CAACCTGGTCCTGGCACTGATTTATAACCTGGTTGGG | |
| | ATACCCATTGCAGCAGGTGTCTTCATGCCCATCGGCA | |
| | TTGTGCTGCAGCCCTGGATGGGCTCAGCGGCCATGG | |
| | CAGCCTCCTCTGTGTCTGTGGTGCTCTCATCCCTGCA | |
| | GCTCAAGTGCTATAAGAAGCCTGACCTGGAGAGGTA | |
| | TGAGGCACAGGCGCATGGCCACATGAAGCCCCTGAC | |
| | GGCATCCCAGGTCAGTGTGCACATAGGCATGGATGA | |
| | CAGGTGGCGGGACTCCCCCAGGGCCACACCATGGGA | |
| | CCAGGTCAGCTATGTCAGCCAGGTGTCGCTGTCCTCC | |
| | CTGACGTCCGACAAGCCATCTCGGCACAGCGCTGCA | |
| | GCAGACGATGATGGGGACAAGTGGTCTCTGCTCCTG | |
| | AATGGCAGGGATGAGGAGCAGTACATCtaacatcacatttaaa | |
| | agcatctcaggtaactatattttgaattttttaaaaaagtaactataatagttattattaaaata | |
| | gcaaagattgaccatttccaagagccatatagaccagcaccgaccactattctaaactatt | |
| | tatgtatgtaaatattagcttttaaaattctcaaaatagttgctgagttgggaaccactattatt | |
| | tctattttgtagatgagaaaatgaagataaacatcaaagcatagattaagtaattttccaaa | |
| | gggtcaaaattcaaaattgaaaccaaagtttcagtgttgcccattgtcctgttctgacttata | |
| | tgatgcggtacacagagccatccaagtaagtgatggctcagcagtggaatactctggg | |
| | aattaggctgaaccacatgaaagagtgctttatagggcaaaaacagttgaatatcagtga | |
| | tttcacatggttcaacctaatagttcaactcatcctttccattggagaatatgatggatctac | |
| | cttctgtgaactttatagtgaagaatctgctattacatttccaatttgtcaacatgctgagcttt | |
| | aataggacttatcttcttatgacaacatttattgaggaacccctagtgatggagttggccac | |
| | tccctctctgcgcgctcgctcgctcactgaggCCgcccgggcAAAgcccgggcg | |
| | gcctcagtgagcgagcgagcgcgcagagagggagtggccaactttttgcaaaagcct | |
| | aggcctccaaaaaagcctcctcactacttctggaatagctcagaggccgaggcggcct | |
| | cggcctctgcataaataaaaaaaattagtcagccatggggcggagaatgggcggaact | |
| | gggcggagttaggggcgggatgggcggagttaggggcgggactatggttgctgacta | |
| | attgagatgcatgctttgcatacttctgcctgctggggagcctggggactttccacacctg | |
| | gttgctgactaattgagatgcatgctttgcatacttctgcctgctggggagcctggggact | |
| | ttccacaccctaactgacacacattccacagctgcattaatgaatcggccaacgcgcgg | |
| | ggagaggcggtttgcgtattgggcgctcttccgcttcctcgctcactgactcgctgcgct | |
| | cggtcgttcggctgcggcgagcggtatcagctcactcaaaggcggtaatacggttatcc | |
| | acagaatcaggggataacgcaggaaagaacatgtgagcaaaaggccagcaaaaggc | |
| | caggaaccgtaaaaaggccgcgttgctggcgtttttccataggctccgcccccctgacg | |
| | agcatcacaaaaatcgacgctcaagtcagaggtggcgaaacccgacaggactataaa | |
| | gataccaggcgtttccccctggaagctccctcgtgcgctctcctgttccgaccctgccgc | |
| | ttaccggatacctgtccgcctttctcccttcgggaagcgtggcgctttctcatagctcacg | |
| | ctgtaggtatctcagttcggtgtaggtcgttcgctccaagctgggctgtgtgcacgaacc | |
| | ccccgttcagcccgaccgctgcgccttatccggtaactatcgtcttgagtccaacccggt | |
| | aagacacgacttatcgccactggcagcagccactggtaacaggattagcagagcgag | |
| | gtatgtaggcggtgctacagagttcttgaagtggtggcctaactacggctacactagaa | |
| | gaacagtatttggtatctgcgctctgctgaagccagttaccttcggaaaaagagttggta | |
| | gctcttgatccggcaaacaaaccaccgctggtagcggtggtttttttgtttgcaagcagca | |
| | gattacgcgcagaaaaaaaggatctcaagaagatcctttgatcttttctacggggtctga | |
| | cgctcagtggaacgaaaactcacgttaagggattttggtcatgagattatcaaaaaggat | |
| | cttcacctagatccttttaaattaaaaatgaagttttaaatcaatctaaagtatatatgagtaa | |
| | acttggtctgacagttagaaaaactcatcgagcatcaaatgaaactgcaatttattcatatc | |
| | aggattatcaataccatatttttgaaaaagccgtttctgtaatgaaggagaaaactcaccg | |
| | aggcagttccataggatggcaagatcctggtatcggtctgcgattccgactcgtccaac | |
| | atcaatacaacctattaatttcccctcgtcaaaaataaggttatcaagtgagaaatcaccat | |
| | gagtgacgactgaatccggtgagaatggcaaaagtttatgcatttctttccagacttgttc | |
| | aacaggccagccattacgctcgtcatcaaaatcactcgcatcaaccaaaccgttattcatt | |
| | cgtgattgcgcctgagcgagacgaaatacgcgatcgctgttaaaaggacaattacaaa | |
| | caggaatcgaatgcaaccggcgcaggaacactgccagcgcatcaacaatattttcacc | |
| | tgaatcaggatattcttctaatacctggaatgctgtttttccggggatcgcagtggtgagta | |
| | accatgcatcatcaggagtacggataaaatgcttgatggtcggaagaggcataaattcc | |
| | gtcagccagtttagtctgaccatctcatctgtaacatcattggcaacgctacctttgccatg | |
| | tttcagaaacaactctggcgcatcgggcttcccatacaagcgatagattgtcgcacctga | |
| | ttgcccgacattatcgcgagcccatttatacccatataaatcagcatccatgttggaattta | |
| | atcgcggcctcgacgtttcccgttgaatatggctcatactcttcctttttcaatattattgaag | |
| | catttatcagggttattgtctcatgagcggatacatatttgaatgtatttagaaaaataaaca | |
| | aataggggttccgcgcacatttccccgaaaagtgccacctgacgtctaagaaaccattat | |
| | tatcatgacattaacctataaaaataggcgtatcacgaggccctttcgtctcgcgcgtttc | |
| | ggtgatgacggtgaaaacctctgacacatgcagctcccggagacggtcacagcttgtct | |
| | gtaagcggatgccgggagcagacaagcccgtcagggcgcgtcagcgggtgttggcg | |
| | ggtgtcggggctggcttaactatgcggcatcagagcagattgtactgagagtgcaccat | |
| | tcgacgctctcccttatgcgactcctgcattaggaagcagcccagtagtaggttgaggc | |
| | cgttgagcaccgccgccgcaaggaatggtgcatgcaaggagatggcgcccaacagt | |
| | cccccggccacggggcctgccaccatacccacgccgaaacaagcgctcatgagccc | |
| | gaagtggcgagcccgatcttccccatcggtgatgtcggcgatataggcgccagcaacc | |
| | gcacctgtggcgccggtgggtcaccaagcaggaagtcaaagactttttccggtgggca | |
| | aaggatcacgtggttgaggtggagcatgaattctacgtcaaaaagggtggagccaaga | |
| | aaagacccgcccccagtgacgcagatataagtgagcccaaacgggtgcgcgagtca | |
| | gttgcgcagccatcgacgtcagacgcggaagcttcgatcaactacgcagacaggtacc | |
| | aaaacaaatgttctcgtcacgtgggcatgaatctgatgctgtttccctgcagacaatgcg | |
| | agagaatgaatcagaattcaaatatctgcttcactcacggacagaaagactgtttagagt | |
| | gctttcccgtgtcagaatctcaacccgtttctgtcgtcaaaaaggcgtatcagaaactgtg | |
| | ctacattcatcatatcatgggaaaggtgccagacgcttgcactgcctgcgatctggtcaa | |
| | tgtggatttggatgactgcatctttgaacaataaatgatttaaatcaggt | |
| SL65-GR | CCCCTGTAAttgcttgttaatcaataaaccgtttaattcgtttcagttgaactttggtc | 43 |
| hATP7B | tctgcgtatttctttcttatctagtttccatatgcatgtagataagtagcatggcgggttaatc | |
| | attaactaaccggtacctctagaactatagctagcgatgaccctgctgattggttcgctga | |
| | ccatttccgggtgcgggacggcgttaccagaaactcagaaggttcgtccaaccaaacc | |
| | gactctgacggcagtttacgagagagatgatagggtctgcttcagtaagccagatgcta | |
| | cacaattaggcttgtacatattgtcgttagaacgcggctacaattaatacataaccttatgt | |
| | atcatacacatacgatttaggtgacactatagaatacacggaattaattcttggccactcc | |
| | ctctctgcgcgctcgctcgctcactgaggccgcccgggcaaagcccgggcgtcgggc | |
| | gacctttggtcgcccggcctcagtgagcgagcgagcgcgcagagagggagtggcca | |
| | actccatcactaggggttcctgcatgtttggttaggctagggcttagggatttatatatcaa | |
| | aggaggctttgtacatgtgggacagggatcttattttacaaacaattgtcttacaaaatgaa | |
| | taaaacagcactttgtttttatctcctgctctattgtgccatactgttaaatgtttataatgcctg | |
| | ttctgtttccaaatttgtgatgcttatgaatattaataggaatatttgtaaggcctgaaatatttt | |
| | gatcatgaaatcaaaacattaatttatttaaacatttacttgaaatgtggtggtttgtgatttag | |
| | ttgattttataggctagtgggagaatttacattcaaatgtctaaatcacttaaaattgcccttt | |
| | atggcctgacagtaacttttttttattcatttggggacaactatgtccgtgagcttccgtcca | |
| | gagattatagtagtaaattgtaattaaaggatatgatgcacgtgaaatcactttgcaatcat | |
| | caatagcttcataaatgttaattttgtatcctaatagtaatgctaatattttcctaacatctgtca | |
| | tgtctttgtgttcagggtaaaaaacttgttgctgcaagtcaagctgccttaggcttaggcag | |
| | cggcgccaccaacttcagcctgctgaaacaggccGGCGACGTGGAAGA | |
| | GAACCCTGGCCCTCCTGAGCAGGAGAGACAGATCAC | |
| | AGCCAGAGAAGGGGCCAGTCGGAAAATCTTATCTAA | |
| | GCTTTCTTTGCCTACCCGTGCCTGGGAACCAGCAATG | |
| | AAGAAGAGTTTTGCTTTTGACAATGTTGGCTATGAAG | |
| | GTGGTCTGGATGGCCTGGGCCCTTCTTCTCAGCCGCA | |
| | GAAGTGCTTCTTACAGATCAAAGGCATGACCTGTGC | |
| | ATCCTGTGTGTCTAACATAGAAAGGAATCTGCAGAA | |
| | AGAAGCTGGTGTTCTCTCCGTGTTGGTTGCCTTGATG | |
| | GCAGGAAAGGCAGAGATCAAGTATGACCCAGAGGTC | |
| | ATCCAGCCCCTCGAGATAGCTCAGTTCATCCAGGACC | |
| | TGGGTTTTGAGGCAGCAGTCATGGAGGACTACGCAG | |
| | GCTCCGATGGCAACATTGAGCTGACAATCACAGGGA | |
| | TGACCTGCGCGTCCTGTGTCCACAACATAGAGTCCAA | |
| | ACTCACGAGGACAAATGGCATCACTTATGCCTCCGTT | |
| | GCCCTTGCCACCAGCAAAGCCCTTGTTAAGTTTGACC | |
| | CGGAAATTATCGGTCCACGGGATATTATCAAAATTAT | |
| | TGAGGAAATTGGCTTTCATGCTTCCCTGGCCCAGAGA | |
| | AACCCCAACGCTCATCACTTGGACCACAAGATGGAA | |
| | ATAAAGCAGTGGAAGAAGTCTTTCCTGTGCAGCCTG | |
| | GTGTTTGGCATCCCTGTCATGGCCTTAATGATCTATA | |
| | TGCTGATACCCAGCAACGAGCCCCACCAGTCCATGG | |
| | TCCTGGACCACAACATCATTCCAGGACTGTCCATTCT | |
| | AAATCTCATCTTCTTTATCTTGTGTACCTTTGTCCAGC | |
| | TCCTCGGTGGGTGGTACTTCTACGTTCAGGCCTACAA | |
| | ATCTCTGAGACACAGGTCAGCCAACATGGACGTGCT | |
| | CATCGTCCTGGCCACAAGCATTGCTTATGTTTATTCT | |
| | CTGGTCATCCTGGTGGTTGCTGTGGCTGAGAAGGCG | |
| | GAGAGGAGCCCTGTGACATTCTTCGACACGCCCCCC | |
| | ATGCTCTTTGTGTTCATTGCCCTGGGCCGGTGGCTGG | |
| | AACACTTGGCAAAGAGCAAAACCTCAGAAGCCCTGG | |
| | CTAAACTCATGTCTCTCCAAGCCACAGAAGCCACCGT | |
| | TGTGACCCTTGGTGAGGACAATTTAATCATCAGGGA | |
| | GGAGCAAGTCCCCATGGAGCTGGTGCAGCGGGGCGA | |
| | TATCGTCAAGGTGGTCCCTGGGGGAAAGTTTCCAGT | |
| | GGATGGGAAAGTCCTGGAAGGCAATACCATGGCTGA | |
| | TGAGTCCCTCATCACAGGAGAAGCCATGCCAGTCAC | |
| | TAAGAAACCCGGAAGCACTGTAATTGCGGGGTCTAT | |
| | AAATGCACATGGCTCTGTGCTCATTAAAGCTACCCAC | |
| | GTGGGCAATGACACCACTTTGGCTCAGATTGTGAAA | |
| | CTGGTGGAAGAGGCTCAGATGTCAAAGGCACCCATT | |
| | CAGCAGCTGGCTGACCGGTTTAGTGGATATTTTGTCC | |
| | CATTTATCATCATCATGTCAACTTTGACGTTGGTGGT | |
| | ATGGATTGTAATCGGTTTTATCGATTTTGGTGTTGTTC | |
| | AGAGATACTTTCCTAACCCCAACAAGCACATCTCCCA | |
| | GACAGAGGTGATCATCCGGTTTGCTTTCCAGACGTCC | |
| | ATCACGGTGCTGTGCATTGCCTGCCCCTGCTCCCTGG | |
| | GGCTGGCCACGCCCACGGCTGTCATGGTGGGCACCG | |
| | GGGTGGCCGCGCAGAACGGCATCCTCATCAAGGGAG | |
| | GCAAGCCCCTGGAGATGGCGCACAAGATAAAGACTG | |
| | TGATGTTTGACAAGACTGGCACCATTACCCATGGCGT | |
| | CCCCAGGGTCATGCGGGTGCTCCTGCTGGGGGATGT | |
| | GGCCACACTGCCCCTCAGGAAGGTTCTGGCTGTGGT | |
| | GGGGACTGCGGAGGCCAGCAGTGAACACCCCTTGGG | |
| | CGTGGCAGTCACCAAATACTGTAAAGAGGAACTTGG | |
| | AACAGAGACCTTGGGATACTGCACGGACTTCCAGGC | |
| | AGTGCCAGGCTGTGGAATTGGGTGCAAAGTCAGCAA | |
| | CGTGGAAGGCATCCTGGCCCACAGTGAGCGCCCTTT | |
| | GAGTGCACCGGCCAGTCACCTGAATGAGGCTGGCAG | |
| | CCTTCCCGCAGAAAAAGATGCAGTCCCCCAGACCTT | |
| | CTCTGTGCTGATTGGAAACCGTGAGTGGCTGAGGCG | |
| | CAACGGTTTAACCATTTCTAGCGATGTCAGTGACGCT | |
| | ATGACAGACCACGAGATGAAAGGACAGACAGCCATC | |
| | CTGGTGGCTATTGACGGTGTGCTCTGTGGGATGATCG | |
| | CAATCGCAGACGCTGTCAAGCAGGAGGCTGCCCTGG | |
| | CTGTGCACACGCTGCAGAGCATGGGTGTGGACGTGG | |
| | TTCTGATCACGGGGGACAACCGGAAGACAGCCAGAG | |
| | CTATTGCCACCCAGGTTGGCATCAACAAAGTCTTTGC | |
| | AGAGGTGCTGCCTTCGCACAAGGTGGCCAAGGTCCA | |
| | GGAGCTCCAGAATAAAGGGAAGAAAGTCGCCATGGT | |
| | GGGGGATGGGGTCAATGACTCCCCGGCCTTGGCCCA | |
| | GGCAGACATGGGTGTGGCCATTGGCACCGGCACGGA | |
| | TGTGGCCATCGAGGCAGCCGACGTCGTCCTTATCAG | |
| | AAATGATTTGCTGGATGTGGTGGCTAGCATTCACCTT | |
| | TCCAAGAGGACTGTCCGAAGGATACGCATCAACCTG | |
| | GTCCTGGCACTGATTTATAACCTGGTTGGGATACCCA | |
| | TTGCAGCAGGTGTCTTCATGCCCATCGGCATTGTGCT | |
| | GCAGCCCTGGATGGGCTCAGCGGCCATGGCAGCCTC | |
| | CTCTGTGTCTGTGGTGCTCTCATCCCTGCAGCTCAAG | |
| | TGCTATAAGAAGCCTGACCTGGAGAGGTATGAGGCA | |
| | CAGGCGCATGGCCACATGAAGCCCCTGACGGCATCC | |
| | CAGGTCAGTGTGCACATAGGCATGGATGACAGGTGG | |
| | CGGGACTCCCCCAGGGCCACACCATGGGACCAGGTC | |
| | AGCTATGTCAGCCAGGTGTCGCTGTCCTCCCTGACGT | |
| | CCGACAAGCCATCTCGGCACAGCGCTGCAGCAGACG | |
| | ATGATGGGGACAAGTGGTCTCTGCTCCTGAATGGCA | |
| | GGGATGAGGAGCAGTACATCtaacatcacatttaaaagcatctcagg | |
| | taactatattttgaattttttaaaaaagtaactataatagttattattaaaatagcaaagattga | |
| | ccatttccaagagccatatagaccagcaccgaccactattctaaactatttatgtatgtaaa | |
| | tattagcttttaaaattctcaaaatagttgctgagttgggaaccactattatttctattttgtag | |
| | atgagaaaatgaagataaacatcaaagcatagattaagtaattttccaaagggtcaaaatt | |
| | caaaattgaaaccaaagtttcagtgttgcccattgtcctgttctgacttatatgatgcggtac | |
| | acagagccatccaagtaagtgatggctcagcagtggaatactctgggaattaggctgaa | |
| | ccacatgaaagagtgctttatagggcaaaaacagttgaatatcagtgatttcacatggttc | |
| | aacctaatagttcaactcatcctttccattggagaatatgatggatctaccttctgtgaacttt | |
| | atagtgaagaatctgctattacatttccaatttgtcaacatgctgagctttaataggacttatc | |
| | ttcttatgacaacatttattgaggaacccctagtgatggagttggccactccctctctgcgc | |
| | gctcgctcgctcactgaggCCgcccgggcAAAgcccgggggcctcagtgagc | |
| | gagcgagcgcgcagagagggagtggccaactttttgcaaaagcctaggcctccaaaa | |
| | aagcctcctcactacttctggaatagctcagaggccgaggcggcctcggcctctgcata | |
| | aataaaaaaaattagtcagccatggggcggagaatgggcggaactgggcggagttag | |
| | gggcgggatgggcggagttaggggcgggactatggttgctgactaattgagatgcatg | |
| | ctttgcatacttctgcctgctggggagcctggggactttccacacctggttgctgactaatt | |
| | gagatgcatgctttgcatacttctgcctgctggggagcctggggactttccacaccctaa | |
| | ctgacacacattccacagctgcattaatgaatcggccaacgcgcggggagaggcggtt | |
| | tgcgtattgggcgctcttccgcttcctcgctcactgactcgctgcgctcggtcgttcggct | |
| | gcggcgagcggtatcagctcactcaaaggcggtaatacggttatccacagaatcaggg | |
| | gataacgcaggaaagaacatgtgagcaaaaggccagcaaaaggccaggaaccgtaa | |
| | aaaggccgcgttgctggcgtttttccataggctccgcccccctgacgagcatcacaaaa | |
| | atcgacgctcaagtcagaggtggcgaaacccgacaggactataaagataccaggcgtt | |
| | tccccctggaagctccctcgtgcgctctcctgttccgaccctgccgcttaccggatacct | |
| | gtccgcctttctcccttcgggaagcgtggcgctttctcatagctcacgctgtaggtatctc | |
| | agttcggtgtaggtcgttcgctccaagctgggctgtgtgcacgaaccccccgttcagcc | |
| | cgaccgctgcgccttatccggtaactatcgtcttgagtccaacccggtaagacacgactt | |
| | atcgccactggcagcagccactggtaacaggattagcagagcgaggtatgtaggcgg | |
| | tgctacagagttcttgaagtggtggcctaactacggctacactagaagaacagtatttggt | |
| | atctgcgctctgctgaagccagttaccttcggaaaaagagttggtagctcttgatccggc | |
| | aaacaaaccaccgctggtagcggtggtttttttgtttgcaagcagcagattacgcgcaga | |
| | aaaaaaggatctcaagaagatcctttgatcttttctacggggtctgacgctcagtggaac | |
| | gaaaactcacgttaagggattttggtcatgagattatcaaaaaggatcttcacctagatcc | |
| | ttttaaattaaaaatgaagttttaaatcaatctaaagtatatatgagtaaacttggtctgacag | |
| | ttagaaaaactcatcgagcatcaaatgaaactgcaatttattcatatcaggattatcaatac | |
| | catatttttgaaaaagccgtttctgtaatgaaggagaaaactcaccgaggcagttccatag | |
| | gatggcaagatcctggtatcggtctgcgattccgactcgtccaacatcaatacaacctatt | |
| | aatttcccctcgtcaaaaataaggttatcaagtgagaaatcaccatgagtgacgactgaa | |
| | tccggtgagaatggcaaaagtttatgcatttctttccagacttgttcaacaggccagccatt | |
| | acgctcgtcatcaaaatcactcgcatcaaccaaaccgttattcattcgtgattgcgcctga | |
| | gcgagacgaaatacgcgatcgctgttaaaaggacaattacaaacaggaatcgaatgca | |
| | accggcgcaggaacactgccagcgcatcaacaatattttcacctgaatcaggatattctt | |
| | ctaatacctggaatgctgtttttccggggatcgcagtggtgagtaaccatgcatcatcag | |
| | gagtacggataaaatgcttgatggtcggaagaggcataaattccgtcagccagtttagtc | |
| | tgaccatctcatctgtaacatcattggcaacgctacctttgccatgtttcagaaacaactct | |
| | ggcgcatcgggcttcccatacaagcgatagattgtcgcacctgattgcccgacattatcg | |
| | cgagcccatttatacccatataaatcagcatccatgttggaatttaatcgcggcctcgacg | |
| | tttcccgttgaatatggctcatactcttcctttttcaatattattgaagcatttatcagggttatt | |
| | gtctcatgagcggatacatatttgaatgtatttagaaaaataaacaaataggggttccgcg | |
| | cacatttccccgaaaagtgccacctgacgtctaagaaaccattattatcatgacattaacc | |
| | tataaaaataggcgtatcacgaggccctttcgtctcgcgcgtttcggtgatgacggtgaa | |
| | aacctctgacacatgcagctcccggagacggtcacagcttgtctgtaagcggatgccg | |
| | ggagcagacaagcccgtcagggcgcgtcagcgggtgttggcgggtgtcggggctgg | |
| | cttaactatgcggcatcagagcagattgtactgagagtgcaccattcgacgctctccctta | |
| | tgcgactcctgcattaggaagcagcccagtagtaggttgaggccgttgagcaccgccg | |
| | ccgcaaggaatggtgcatgcaaggagatggcgcccaacagtcccccggccacgggg | |
| | cctgccaccatacccacgccgaaacaagcgctcatgagcccgaagtggcgagcccg | |
| | atcttccccatcggtgatgtcggcgatataggcgccagcaaccgcacctgtggcgccg | |
| | gtgggtcaccaagcaggaagtcaaagactttttccggtgggcaaaggatcacgtggttg | |
| | aggtggagcatgaattctacgtcaaaaagggtggagccaagaaaagacccgccccca | |
| | gtgacgcagatataagtgagcccaaacgggtgcgcgagtcagttgcgcagccatcga | |
| | cgtcagacgcggaagcttcgatcaactacgcagacaggtaccaaaacaaatgttctcgt | |
| | cacgtgggcatgaatctgatgctgtttccctgcagacaatgcgagagaatgaatcagaa | |
| | ttcaaatatctgcttcactcacggacagaaagactgtttagagtgctttcccgtgtcagaat | |
| | ctcaacccgtttctgtcgtcaaaaaggcgtatcagaaactgtgctacattcatcatatcatg | |
| | ggaaaggtgccagacgcttgcactgcctgcgatctggtcaatgtggatttggatgactg | |
| | catctttgaacaataaatgatttaaatcaggtATGGCTGCtGAcGGTTATC | |
| | TTCCAGATTGGCTCGAGGACACTCTCTCTGAAGGCAT | |
| | TCGCGAGTGGTGGGCGCTGAAACCTGGAGCTCCACA | |
| | ACCCAAGGCCAACCAACAGCATCAGGACAACGGCAG | |
| | GGGTCTTGTGCTTCCTGGGTACAAGTACCTCGGACCC | |
| | TTCAACGGACTCGACAAGGGAGAGCCGGTCAACGAG | |
| | GCAGACGCCGCGGCCCTCGAGCACGACAAGGCCTAC | |
| | GACAAGCAGCTCGAGCAGGGGGACAACCCGTACCTC | |
| | AAGTACAACCACGCCGACGCCGAGTTTCAGGAGCGT | |
| | CTGCAAGAAGATACGTCTTTTGGGGGCAACCTCGGG | |
| | CGAGCAGTCTTCCAGGCCAAGAAGCGGGTTCTCGAA | |
| | CCTCTCGGTCTGGTTGAGGAAGGCGCTAAGACGGCT | |
| | CCTGGAAAGAAGAGACCGGTAGAGCCGTCACCTCAG | |
| | CGTTCCCCCGACTCCTCCACGGGCATCGGCAAGAAA | |
| | GGCCAGCAGCCCGCCAGAAAGAGACTCAATTTCGGT | |
| | CAGACTGGCGACTCAGAGTCAGTCCCCGACCCTCAA | |
| | CCTCTCGGAGAACCTCCAGCAGCGCCCTCTAGTGTGG | |
| | GATCTGGTACAGTGGCTGCAGGCGGTGGCGCACCAA | |
| | TGGCAGACAATAACGAAGGTGCCGACGGAGTGGGTA | |
| | ATGCCTCAGGAAATTGGCATTGCGATTCCACATGGCT | |
| | GGGCGACAGAGTCATTACCACCAGCACCCGAACCTG | |
| | GGCCCTGCCCACCTACAACAACCACCTCTACAAGCA | |
| | AATCTCCAGCCAATCAGGAGCTTCAAACGACAACCA | |
| | CTACTTTGGCTACAGCACCCCTTGGGGGTATTTTGAC | |
| | TTTAACAGATTCCACTGCCACTTCTCACCACGTGACT | |
| | GGCAGCGACTCATTAACAACAACTGGGGATTCCGGC | |
| | CCAAGAGACTCAACTTCAAGCTCTTCAACATCCAAGT | |
| | CAAGGAGGTCACGACGAATGATGGCGTCACGACCAT | |
| | CGCTAATAACCTTACCAGCACGGTTCAAGTCTTCTCG | |
| | GACTCGGAGTACCAGTTGCCGTACGTCCTCGGCTCTG | |
| | CGCACCAGGGCTGCCTCCCTCCGTTCCCGGCGGACGT | |
| | GTTCATGATTCCCCAGTACGGCTACCTAACACTCAAC | |
| | AACGGTAGTCAGGCCGTGGGACGCTCCTCCTTTTACT | |
| | GCCTGGAATATTTCCCATCGCAGATGCTGAGAACGG | |
| | GCAATAACTTTGAGTTCAGCTACAGCTTCGAGGACGT | |
| | GCCTTTCCACAGCAGCTACGCACACAGCCAGAGCTT | |
| | GGACCGACTGATGAATCCTCTCATTGACCAGTACCTG | |
| | TACTACTTATCCAGAACTCAGTCCACAGGAGGAACT | |
| | CAAGGTACCCAGCAATTGTTATTTTCTCAAGCTGGGC | |
| | CTGCAAACATGTCGGCTCAGGCCAAGAACTGGCTGC | |
| | CTGGACCTTGCTACCGGCAGCAGCGAGTCTCCACGA | |
| | CACTGTCGCAAAACAACAACAGCAACTTTGCTTGGA | |
| | CTGGTGCCACCAAATATCACCTGAACGGCAGAAACT | |
| | CGTTGGTTAATCCCGGCGTCGCCATGGCAACTCACAA | |
| | GGACGACGAGGACCGCTTTTTCCCATCCAGCGGAGT | |
| | CCTGATTTTTGGAAAAACTGGAGCAACTAACAAAAC | |
| | TACATTGGAAAATGTGTTAATGACAAATGAAGAAGA | |
| | AATTCGTCCTACTAATCCTGTAGCCACGGAAGAATAC | |
| | GGGATAGTCAGCAGCAACTTACAAGCGGCTAATACT | |
| | GCAGCCCAGACACAAGTTGTCAACAACCAGGGAGCC | |
| | TTACCTGGCATGGTCTGGCAGAACCGGGACGTGTAC | |
| | CTGCAGGGTCCCATTTGGGCCAAAATTCCTCACACAG | |
| | ATGGACACTTTCACCCGTCTCCTCTTATGGGCGGCTT | |
| | TGGACTCAAGAACCCGCCTCCTCAGATCCTCATCAAA | |
| | AACACGCCTGTTCCTGCGAATCCTCCGGCGGAGTTTT | |
| | CAGCTACAAAGTTTGCTTCATTCATCACCCAGTATTC | |
| | CACAGGACAAGTGAGCGTGGAGATTGAATGGGAGCT | |
| | GCAGAAAGAAAACAGCAAACGCTGGAATCCCGAAG | |
| | TGCAGTATACATCTAACTATGCAAAATCTGCCAACGT | |
| | TGATTTCACTGTGGACAACAATGGACTTTATACTGAG | |
| | CCTCGCCCCATTGGCACCCGTTACCTTACCCGT | |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A composition comprising:
a closed circular cDNA integrating gene therapy construct comprising, from 5′ to 3′, a polynucleotide sequence encoding (a) a 5′ homology arm between 0.4 kb and 0.8 kb in length, (b) a P2A coding sequence encoding a P2A peptide, (c) a therapeutic payload, and (d) a 3′ homology arm between 0.4 kb and 0.8 kb in length, wherein:
the therapeutic payload comprises a transgene sequence encoding ATP7B or a variant thereof; and
the homology arm sequences promote integration of the construct at an endogenous albumin target site such that the albumin locus can result in the simultaneous production of albumin-2A and the transgene as separate proteins.

2. The composition of claim 1, wherein the closed circular cDNA integrating gene therapy construct comprises the nucleotide sequence of SEQ ID NO: 34, SEQ ID NO: 35, or SEQ ID NO: 36.

3. (canceled)

4. The composition of claim 1, wherein;
(a) the 5′ homology arm sequence comprises the nucleotide sequence of SEQ ID NO:6, SEQ ID NO: 7, or SEQ ID NO:8; and/or
(b) the 3′ homology arm sequence comprises the nucleotide sequence of SEQ ID NO: 9, SEQ ID NO: 10, or SEQ ID NO: 11.

5. (canceled)

6. The composition of claim, wherein;
(a) the P2A coding sequence comprises the nucleotide sequence of SEQ ID NO: 16 or SEQ ID NO: 17; and/or
(b) the P2A coding sequence encodes a peptide comprising the amino acid sequence of SEQ ID NO: 18.

7. (canceled)

8. The composition of claim 1, wherein the transgene sequence encoding ATP7B or a variant thereof comprises the nucleotide sequence of SEQ ID NO: 15.

9. (canceled)

10. The composition of claim 1, wherein the composition further comprises an adeno-associated viral (AAV) capsid protein.

11. The composition of claim 10, wherein the AAV capsid protein comprises an amino acid sequence having at least 95% sequence identity to the amino acid sequence of AAV8, AAV-DJ, AAV-LK03, sL65, or AAVNP59.

12. A method of treating Wilson's Disease comprising administering to a subject a dose of the composition of claim 1.

13. The method of claim 12, wherein the closed circular cDNA integrating gene therapy construct comprises the nucleotide sequence of SEQ ID NO: 34, SEQ ID NO: 35, or SEQ ID NO: 36.

14. (canceled)

15. The method of claim 12, wherein;
(a) the 5′ homology arm comprises the nucleotide sequence of SEQ ID NO:6, SEQ ID NO: 7, or SEQ ID NO:8; and/or
(b) the 3′ homology arm comprises the nucleotide sequence of SEQ ID NO: 9, SEQ ID NO: 10, or SEQ ID NO: 11.

16. (canceled)

17. The method of claim 12, wherein:
(a) the P2A coding sequence comprises the nucleotide sequence of SEQ ID NO: 16 or SEQ ID NO: 17; and/or
(b) the P2A coding sequence encodes a peptide comprising the amino acid sequence of SEQ ID NO: 18.

18. (canceled)

19. The method of claim 12, wherein the transgene sequence encoding ATP7B or a variant thereof comprises the nucleotide sequence of SEQ ID NO: 15.

20. (canceled)

21. The method of claim 12, wherein the composition further comprises an AAV capsid protein.

22. The method of claim 21, wherein the AAV capsid protein comprises an amino acid sequence having at least 95% sequence identity to the amino acid sequence of; AAV8, AAV-DJ; AAV-LK03; sL65; or AAVNP59.

23. The method of claim 12, wherein:
(a) the composition is administered to the subject in dosages between 1E12 and 1E14 vg/kg;
(b) the composition is administered to the subject in dosages between 3E12 and 1E13 vq/kg;
(c) the composition is administered to the subject in dosages between 3E12 and 3E13 vq/kg
(d) the composition is administered to the subject in dosages of no more than 3E13 vq/kg; or
(e) the composition is administered to the subject in dosages of no more than 3E12 vq/kg.

24-27. (canceled)

28. The method of claim 12, wherein:
(a) the composition is administered to the subject only once;
(b) the composition is administered to the subject more than once;
(c) the subject is a newborn;
(d) the subject is between 0 days and 1 month of age;
(e) the subject is between 3 months of age and 1 year of age;
(f) the subject is between 1 year of age and 5 years of age; or
(g) the subject is 5 years of age or older.

29-32. (canceled)

33. A liver-targeted, recombinant AAV vector for treating Wilson's Disease and encoding the therapeutic transgene ATP7B, the viral vector comprising a closed, circular cDNA polynucleotide sequence comprising
an ATP7B polynucleotide sequence encoding a functional ATP7B therapeutic transgene comprising the nucleotide sequence of SEQ ID NO:15, preceded by a 2A-peptide sequence encoding a 2A-peptide comprising the amino acid sequence of SEQ ID NO: 18;
the ATP7B polynucleotide sequence and 2A-peptide sequence together flanked by a 3′ homology arm comprising the nucleotide sequence of SEQ ID NO: 9, SEQ ID NO: 10, or SEQ ID NO: 11; and a 5′ homology arm comprising the nucleotide sequence of SEQ ID NO:6, SEQ ID NO: 7, or SEQ ID NO:8.

34-38. (canceled)

39. The method of claim 12, wherein the closed circular cDNA integrating gene therapy construct is a liver-targeted, recombinant AAV.

40-43. (canceled)

44. A method of treating Wilson's Disease comprising administering to a subject in need thereof a therapeutically effective dose of a liver-targeted, recombinant AAV vector encoding the therapeutic transgene ATP7B, the viral vector comprising a cDNA polynucleotide sequence comprising
an ATP7B polynucleotide sequence encoding a functional ATP7B therapeutic transgene comprising the nucleotide sequence of SEQ ID NO:15, preceded by a 2A-peptide sequence encoding a 2A-peptide comprising the amino acid sequence of SEQ ID NO: 18;
the ATP7B polynucleotide sequence and 2A-peptide sequence together flanked by a 3′ homology arm comprising the nucleotide sequence of SEQ ID NO: 9, SEQ ID NO: 10, or SEQ ID NO: 11; and a 5′ homology arm comprising the nucleotide sequence of SEQ ID NO:6, SEQ ID NO: 7, or SEQ ID NO:8.

45. A method of reducing the level of non-ceruloplasmin-bound copper in a subject in need thereof, the method comprising administering to a subject in need thereof a liver-targeted, recombinant AAV vector encoding the therapeutic transgene ATP7B in an amount effective to reduce the level of non-ceruloplasmin-bound copper in the subject, the liver-targeted, recombinant AAV vector comprising a cDNA polynucleotide sequence expressing a functional therapeutic ATP7B transgene preceded by a 2A-peptide coding sequence and flanked by 0.4 kb and 0.8 kb gene homology arms spanning the albumin stop codon, wherein the homology arm sequences promote integration of the construct at an endogenous albumin target site, such that the albumin locus can result in the simultaneous production of albumin-2A and the ATP7B transgene as separate proteins.

46. (canceled)


SEQUENCE LISTING (58 sequences)

SEQ ID NO: 1 - DNA, 1159 bp, Mus musculus
  misc_feature 1..1159: note = Mouse albumin 1 kb 5 homology arm
  source 1..1159: mol_type = genomic DNA
gtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctaccgagactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagcc

SEQ ID NO: 2 - DNA, 623 bp, Mus musculus
  misc_feature 1..623: note = Mouse albumin 0.6 kb 5 homology arm version 1
  source 1..623: mol_type = genomic DNA
tgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagcc

SEQ ID NO: 3 - DNA, 615 bp, Mus musculus
  misc_feature 1..615: note = Mouse albumin 0.6 kb 5 homology arm version 2
  source 1..615: mol_type = genomic DNA
gactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagcc

SEQ ID NO: 4 - DNA, 615 bp, Mus musculus
  misc_feature 1..615: note = Mouse albumin 0.6 kb 3 homology arm version 1
  source 1..615: mol_type = genomic DNA
taaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatg

SEQ ID NO: 5 - DNA, 622 bp, Mus musculus
  misc_feature 1..622: note = Mouse albumin 0.6 kb 3 homology arm version 2
  source 1..622: mol_type = genomic DNA
taaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatca

SEQ ID NO: 6 - DNA, 427 bp, Homo sapiens
  misc_feature 1..427: note = Human albumin 0.4 kb 5 homology arm
  source 1..427: mol_type = genomic DNA
gtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 7 - DNA, 612 bp, Homo sapiens
  misc_feature 1..612: note = Human albumin 0.6 kb 5 homology arm
  source 1..612: mol_type = genomic DNA
gcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 8 - DNA, 800 bp, Homo sapiens
  misc_feature 1..800: note = Human albumin 0.8 kb 5 homology arm
  source 1..800: mol_type = genomic DNA
ttcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggctta

SEQ ID NO: 9 - DNA, 424 bp, Homo sapiens
  misc_feature 1..424: note = Human albumin 0.4 kb 3 homology arm
  source 1..424: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttata

SEQ ID NO: 10 - DNA, 612 bp, Homo sapiens
  misc_feature 1..612: note = Human albumin 0.6 kb 3 homology arm
  source 1..612: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattg

SEQ ID NO: 11 - DNA, 807 bp, Homo sapiens
  misc_feature 1..807: note = Human albumin 0.8 kb 3 homology arm
  source 1..807: mol_type = genomic DNA
taacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgag

SEQ ID NO: 12 - DNA, 609 bp, Macaca fascicularis
  misc_feature 1..609: note = Cynomolgus albumin 0.6 kb 5 homology arm
  source 1..609: mol_type = genomic DNA
gcatgtttggttaggctacggcttagggatttatatatcaaaggagactttgtacaagtgggacagggatcttattttacaaacaattgtcttacaaaatgaataaaataacactttgtttttatctcctgctctattgtgccatactattaaacgtttataatgcccgttctgtttccaaatttgtgatacttatgaatattaataggaatatttgtaaggcctaaaatattttgattatgaaatcaaaacattaatttatttaaacattttcatgaaaagtggtggtttgtggtttagttgattttatagattagtgggagaatttacattcaaatgtctaaatcacttaaaattgccccttatggcctgacagtatttttttttaattcctttgggaacaactatgtccgtgagcttccatccagagattatagtagtaaattggaattaaaggatatgatgcacgtgaaatcactttgcaatcatcaatagcttcataaatgttaattttgtatcctaatagtaatgctaatattttcctaacatctgtcatgtctttgtattcagggtccaaaatttgttgctgcaagtcaagctgccttagcc

SEQ ID NO: 13 - DNA, 612 bp, Macaca fascicularis
  misc_feature 1..612: note = Cynomolgus albumin 0.6 kb 3 homology arm
  source 1..612: mol_type = genomic DNA
taaaaacatcacaattaagaacatctcaggtaactatattttgaattttttaaaaaagtaactataacagttattattaaaatagcaaagattgactgacgatttccaagagccatacagaccagcaccaaccactattctaaactatttatatatgtacatattagcttttaaaattctcaaaatagttgctgagttgggaaccactattatttctattttgtaaatgagaaaatgaagataaacatcaaagcataggttaaataattttccaaagggtcaaaattcaaaattcaaaccaaagtttcagtgttgcccattgtcctattttgacttatatgatgtggcacacagagccatccaagtaagtgatggctcagcaggagaatactctaggaattagactgaaccatatgtaagagcgctttataggacaaaaacagttgaatatcaatgatttcacatggatcaacctaatagttcaactcatcctttccgttggagaatatgatggatctaccttctgtgaactttatagtgaacaatctgctattacattttcaatttgtcaacatgctgaactttaataggacttattttcttatgacaaaa

SEQ ID NO: 14 - DNA, 3084 bp, Mus musculus
  misc_feature 1..3084: note = Mouse truncated ATP7B
  source 1..3084: mol_type = genomic DNA
cctgaacaggagagacaggtcacagccaaagaggccagtcggaaaatcttatctaaacttgctttgcccggccggccctgggagcaatcaatgaagcagagcttcgccttcgacaatgttggctacgaagggggtctggacagcaccagctcgtccccatcacagaagtgcttcgtacagatcaaaggcatgacctgtgcgtcctgtgtgtctaacatagaaaggagtctccagagacatgctggtattctctcagtgttggtcgccttgatgtcgggaaaggcagaggtcaagtatgatccggagatcatccagtcgcccaggatagctcagctcatccaggacctgggcttcgaagcgtcagtcatggaggacaacacagtctctgaaggtgacatcgaactgattatcacagggatgacctgtgcttcctgtgtccacaacatagagtccaagctcacaaggacaaatggcatcacttacgcctctgtggcccttgccaccagcaaagcccatgtgaagttcgatcctgaaattgttggtccccgtgacatcatcaagatcattgaggaaattggctttcatgcttccctggcccagagaaaccccaacgcccatcacttggaccacaagacggaaataaaacagtggaagaagtctttcctgtgcagcctggtgttcggcatccccgtcatgggattgatggtctacatgttaatccccagcagtacgcctcaggagacgatggtcctggaccacaacatcatcccaggactgtccgttctcaatctcatcttcttcatcttgtgtacctttgtccaatttctgggtgggtggtacttctacgtacaagcctacaaatcgctgagacacaggtccgccaacatggacgtactcatcgtgctcgccacaaccattgcctatgcctactccctggtcatcctggtggtcgccgtagccgagaaggcagagaagagccccgtgaccttctttgacacgccccccatgctctttgtgttcatcgccctgggacggtggctggaacacgtggccaagagcaaaacttcagaagcccttgcaaaactcatgtcactccaagccacagaagccacagtcgtgaccctgggtgaggacaacttaatcctcagagaggagcaggtgcccatggagctggtgcagcgaggcgacgtcatcaaggttgtccctgggggcaagttcccagtggatgggaaagtcctcgaaggcaataccatggctgatgagtccctcatcacaggagaggccatgcctgtcactaagaaacctgggagcatagtgattgctggctctattaatgctcatggctctgtgctccttaaagctacccatgtgggtaatgacacaactttggctcagattgtaaagttggtggaagaggcccagatgtcaaaggctcccattcagcagctggctgaccggttcagtggatattttgtcccattcatcatcatcatttcaaccttgaccctggtggtgtggatcgtcattggctttgtcgatttcggtgtggttcagaagtactttcctagccctagcaagcacatctcgcagacagaggtgatcatccgctttgccttccagacgtccatcactgtgctgtgcatcgcctgcccctgctccctggggctggccacacccacagcagtcatggtgggcactggggtggctgcccagaacggtgtcctaatcaaaggagggaagcctctggagatggcacacaagataaagaccgttatgtttgacaaaacgggcaccatcacccacggggtccccagagtcatgcggttcctgctgctcgcagacgtggccacactccccctcaggaaggttctggccgtggtgggcaccgcggaggccagcagcgagcaccccttaggcgtggccgtcactaaatactgcaaagaggaacttgggacggagaccctgggatacagcacagacttccaggcagtgcccggctgtggaattagctgcaaagttagcaacgtggagggcatcctggctcgcagtgatctgactgctcaccctgttggagttggcaaccctcccacaggggaaggtgcaggtccccagaccttctccgtgctgattggaaaccgggaatggatgcggcgaaacggtttaaccatctccagtgacatcagtgacgccatgacagatcacgagatgaaaggacagacggccatcctggtggccattgatggtgtgctctgcgggatgatcgccatcgcagatgctgtcaaaccagaggctgccctggctatctacaccctgaaaagcatgggtgtggatgtggctctgatcacaggggacaaccggaagacagccagagccattgctactcaggttggcatcaacaaagtctttgcggaggtactgccttctcacaaggtggccaaggtccaggagcttcagaatgaagggaagaaagtcgccatggtgggagatggggtgaatgactccccagccctggcccaggctgacgtgggcatcgccatcgggactggcacagatgttgccatcgaagcagcagacgtggtcctgatcagaaatgacttattggacgtcgtggccagcattcatctctccaagaggaccgtccggaggatccgggtcaatctggtgctggcattgatttataacatggttgggatacctattgctgcaggtgtcttcatgcccattggcatcgtgctgcagccgtggatgggctcagcagccatggctgcgtcctctgtctctgtggtgctctcgtctcttcagctcaagtgctatagaaagcccgacctagagagatatgaggcccaggcccacggccgcatgaagcccctgagtgcctcccaagtcagcgtgcacattggcatggatgaccggcgtcgggattctcccagggccaccgcgtgggaccaggtcagctacgtgagccaagtgtctctgtcctccctgacgtcagacagattgtctcggcatggcggggcagcagaggacggtggcgacaaatggtccctgctcctgagtgacagggatgaagagcagtgcatctga

SEQ ID NO: 15 - DNA, 3102 bp, Homo sapiens
  misc_feature 1..3102: note = Human truncated ATP7B
  source 1..3102: mol_type = genomic DNA
cctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatc

SEQ ID NO: 16 - DNA, 66 bp, Porcine teschovirus-1
  misc_feature 1..66: note = P2A nucleotide sequence version 1
  source 1..66: mol_type = genomic DNA
ggaagcggcgccaccaatttcagcctgctgaaacaggccggcgacgtggaagagaaccctggccct

SEQ ID NO: 17 - DNA, 66 bp, Porcine teschovirus-1
  misc_feature 1..66: note = P2A nucleotide sequence version 2
  source 1..66: mol_type = genomic DNA
ggcagcggcgccaccaacttcagcctgctgaaacaggccggcgacgtggaagagaaccctggccct

SEQ ID NO: 18 - protein, 22 aa, Porcine teschovirus-1
  REGION 1..22: note = misc_feature - P2A peptide sequence
GSGATNFSLLKQAGDVEENPGP

SEQ ID NO: 19 - DNA, 2211 bp, synthetic construct
  misc_feature 1..2211: note = Synthetic: AAV LK03 (nucleotide)
  source 1..2211: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 20 - protein, 736 aa, synthetic construct
  REGION 1..736: note = Synthetic: AAV-LK03 (protein)
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 21 - DNA, 2217 bp, synthetic construct
  misc_feature 1..2217: note = Synthetic: AAV8 (nucleotide)
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctagtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 22 - protein, 738 aa, synthetic construct
  REGION 1..738: note = Synthetic: AAV8 (protein)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 23 - DNA, 2214 bp, synthetic construct
  misc_feature 1..2214: note = Synthetic: AAV-DJ (nucleotide)
  source 1..2214: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 24 - protein, 737 aa, synthetic construct
  REGION 1..737: note = Synthetic: AAV-DJ (protein)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 25 - DNA, 2208 bp, synthetic construct
  misc_feature 1..2208: note = Synthetic: AAV-sL65 (nucleotide)
  source 1..2208: mol_type = other DNA
atggctgccgatggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgtcccctgtaa

SEQ ID NO: 26 - protein, 735 aa, synthetic construct
  REGION 1..735: note = Synthetic: AAV-sL65 (protein)
MAADGYLPDWLEDTLSEGIREWWALKPGAPQPKANQQHQDNGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYSFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - DNA, 2208 bp, synthetic construct
  misc_feature 1..2208: note = Synthetic: AAV-NP59 (nucleotide)
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtatggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactcaacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 28 - protein, 735 aa, synthetic construct
  REGION 1..735: note = Synthetic: AAV-NP59 (protein)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWAGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - DNA, 145 bp, synthetic construct
  misc_feature 1..145: note = Synthetic: 145bp ITR
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 30 - DNA, 130 bp, synthetic construct
  misc_feature 1..130: note = Synthetic: 130bp ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 123 bp, synthetic construct
  misc_feature 1..123: note = Synthetic: B loop deletion ITR
  source 1..123: mol_type = other DNA
aggaacccctagtgatggagttggccactccctctctgcgcgctcgctcgctcactgaggccgcccgggctttgcccgggcggcctcagtgagcgagcgagcgcgcagagagggagtggccaa

SEQ ID NO: 32 - DNA, 123 bp, synthetic construct
  misc_feature 1..123: note = Synthetic: C loop deletion ITR
  source 1..123: mol_type = other DNA
aggaacccctagtgatggagttggccactccctctctgcgcgctcgctcgctcactgaggccgggcgaccaaaggtcgcccggcctcagtgagcgagcgagcgcgcagagagggagtggccaa

SEQ ID NO: 33 - protein, 1035 aa, synthetic construct
  REGION 1..1035: note = Synthetic: codon-optimized variant of a sequence encoding ATP7B
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 34 - DNA, 4402 bp, synthetic construct
  misc_feature 1..4402: note = Synthetic: recombinant viral vector
  source 1..4402: mol_type = other DNA
gtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgag

SEQ ID NO: 35 - DNA, 4392 bp, synthetic construct
  misc_feature 1..4392: note = Synthetic: recombinant viral vector
  source 1..4392: mol_type = other DNA
gcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattg

SEQ ID NO: 36 - DNA, 4392 bp, synthetic construct
  misc_feature 1..4392: note = Synthetic: recombinant viral vector
  source 1..4392: mol_type = other DNA
ttcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttata

SEQ ID NO: 37 - DNA, 4398 bp, synthetic construct
  misc_feature 1..4398: note = Synthetic: recombinant viral vector
  source 1..4398: mol_type = other DNA
gactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatg

SEQ ID NO: 38 - DNA, 4942 bp, synthetic construct
  misc_feature 1..4942: note = Synthetic: recombinant viral vector
  source 1..4942: mol_type = other DNA
gtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctaccgagactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatg

SEQ ID NO: 39 - DNA, 4389 bp, synthetic construct
  misc_feature 1..4389: note = Synthetic: recombinant viral vector
  source 1..4389: mol_type = other DNA
gcatgtttggttaggctacggcttagggatttatatatcaaaggagactttgtacaagtgggacagggatcttattttacaaacaattgtcttacaaaatgaataaaataacactttgtttttatctcctgctctattgtgccatactattaaacgtttataatgcccgttctgtttccaaatttgtgatacttatgaatattaataggaatatttgtaaggcctaaaatattttgattatgaaatcaaaacattaatttatttaaacattttcatgaaaagtggtggtttgtggtttagttgattttatagattagtgggagaatttacattcaaatgtctaaatcacttaaaattgccccttatggcctgacagtatttttttttaattcctttgggaacaactatgtccgtgagcttccatccagagattatagtagtaaattggaattaaaggatatgatgcacgtgaaatcactttgcaatcatcaatagcttcataaatgttaattttgtatcctaatagtaatgctaatattttcctaacatctgtcatgtctttgtattcagggtccaaaatttgttgctgcaagtcaagctgccttagccggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaaaaacatcacaattaagaacatctcaggtaactatattttgaattttttaaaaaagtaactataacagttattattaaaatagcaaagattgactgacgatttccaagagccatacagaccagcaccaaccactattctaaactatttatatatgtacatattagcttttaaaattctcaaaatagttgctgagttgggaaccactattatttctattttgtaaatgagaaaatgaagataaacatcaaagcataggttaaataattttccaaagggtcaaaattcaaaattcaaaccaaagtttcagtgttgcccattgtcctattttgacttatatgatgtggcacacagagccatccaagtaagtgatggctcagcaggagaatactctaggaattagactgaaccatatgtaagagcgctttataggacaaaaacagttgaatatcaatgatttcacatggatcaacctaatagttcaactcatcctttccgttggagaatatgatggatctaccttctgtgaactttatagtgaacaatctgctattacattttcaatttgtcaacatgctgaactttaataggacttattttcttatgacaaaa

SEQ ID NO: 40 - DNA, 4395 bp, synthetic construct
  misc_feature 1..4395: note = Synthetic: recombinant viral vector
  source 1..4395: mol_type = other DNA
tgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggccctcctgaacaggagagacaggtcacagccaaagaggccagtcggaaaatcttatctaaacttgctttgcccggccggccctgggagcaatcaatgaagcagagcttcgccttcgacaatgttggctacgaagggggtctggacagcaccagctcgtccccatcacagaagtgcttcgtacagatcaaaggcatgacctgtgcgtcctgtgtgtctaacatagaaaggagtctccagagacatgctggtattctctcagtgttggtcgccttgatgtcgggaaaggcagaggtcaagtatgatccggagatcatccagtcgcccaggatagctcagctcatccaggacctgggcttcgaagcgtcagtcatggaggacaacacagtctctgaaggtgacatcgaactgattatcacagggatgacctgtgcttcctgtgtccacaacatagagtccaagctcacaaggacaaatggcatcacttacgcctctgtggcccttgccaccagcaaagcccatgtgaagttcgatcctgaaattgttggtccccgtgacatcatcaagatcattgaggaaattggctttcatgcttccctggcccagagaaaccccaacgcccatcacttggaccacaagacggaaataaaacagtggaagaagtctttcctgtgcagcctggtgttcggcatccccgtcatgggattgatggtctacatgttaatccccagcagtacgcctcaggagacgatggtcctggaccacaacatcatcccaggactgtccgttctcaatctcatcttcttcatcttgtgtacctttgtccaatttctgggtgggtggtacttctacgtacaagcctacaaatcgctgagacacaggtccgccaacatggacgtactcatcgtgctcgccacaaccattgcctatgcctactccctggtcatcctggtggtcgccgtagccgagaaggcagagaagagccccgtgaccttctttgacacgccccccatgctctttgtgttcatcgccctgggacggtggctggaacacgtggccaagagcaaaacttcagaagcccttgcaaaactcatgtcactccaagccacagaagccacagtcgtgaccctgggtgaggacaacttaatcctcagagaggagcaggtgcccatggagctggtgcagcgaggcgacgtcatcaaggttgtccctgggggcaagttcccagtggatgggaaagtcctcgaaggcaataccatggctgatgagtccctcatcacaggagaggccatgcctgtcactaagaaacctgggagcatagtgattgctggctctattaatgctcatggctctgtgctccttaaagctacccatgtgggtaatgacacaactttggctcagattgtaaagttggtggaagaggcccagatgtcaaaggctcccattcagcagctggctgaccggttcagtggatattttgtcccattcatcatcatcatttcaaccttgaccctggtggtgtggatcgtcattggctttgtcgatttcggtgtggttcagaagtactttcctagccctagcaagcacatctcgcagacagaggtgatcatccgctttgccttccagacgtccatcactgtgctgtgcatcgcctgcccctgctccctggggctggccacacccacagcagtcatggtgggcactggggtggctgcccagaacggtgtcctaatcaaaggagggaagcctctggagatggcacacaagataaagaccgttatgtttgacaaaacgggcaccatcacccacggggtccccagagtcatgcggttcctgctgctcgcagacgtggccacactccccctcaggaaggttctggccgtggtgggcaccgcggaggccagcagcgagcaccccttaggcgtggccgtcactaaatactgcaaagaggaacttgggacggagaccctgggatacagcacagacttccaggcagtgcccggctgtggaattagctgcaaagttagcaacgtggagggcatcctggctcgcagtgatctgactgctcaccctgttggagttggcaaccctcccacaggggaaggtgcaggtccccagaccttctccgtgctgattggaaaccgggaatggatgcggcgaaacggtttaaccatctccagtgacatcagtgacgccatgacagatcacgagatgaaaggacagacggccatcctggtggccattgatggtgtgctctgcgggatgatcgccatcgcagatgctgtcaaaccagaggctgccctggctatctacaccctgaaaagcatgggtgtggatgtggctctgatcacaggggacaaccggaagacagccagagccattgctactcaggttggcatcaacaaagtctttgcggaggtactgccttctcacaaggtggccaaggtccaggagcttcagaatgaagggaagaaagtcgccatggtgggagatggggtgaatgactccccagccctggcccaggctgacgtgggcatcgccatcgggactggcacagatgttgccatcgaagcagcagacgtggtcctgatcagaaatgacttattggacgtcgtggccagcattcatctctccaagaggaccgtccggaggatccgggtcaatctggtgctggcattgatttataacatggttgggatacctattgctgcaggtgtcttcatgcccattggcatcgtgctgcagccgtggatgggctcagcagccatggctgcgtcctctgtctctgtggtgctctcgtctcttcagctcaagtgctatagaaagcccgacctagagagatatgaggcccaggcccacggccgcatgaagcccctgagtgcctcccaagtcagcgtgcacattggcatggatgaccggcgtcgggattctcccagggccaccgcgtgggaccaggtcagctacgtgagccaagtgtctctgtcctccctgacgtcagacagattgtctcggcatggcggggcagcagaggacggtggcgacaaatggtccctgctcctgagtgacagggatgaagagcagtgcatctgataaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatca

SEQ ID NO: 41 - DNA, 4392 bp, synthetic construct; identical to SEQ ID NO: 35
  misc_feature 1..4392: note = Synthetic: Gene of interest (GOI) cassette
  source 1..4392: mol_type = other DNA

SEQ ID NO: 42 - DNA, 10557 bp, synthetic construct
  misc_feature 1..10557: note = Synthetic: LK03-GR hATP7B
  source 1..10557: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattgaggaacccctagtgatggagttggccactccctctctgcgcgctcgctcgctcactgaggccgcccgggcaaagcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 43 - DNA, 10554 bp, synthetic construct
  misc_feature 1..10554: note = Synthetic: sL65-GR hATP7B
  source 1..10554: mol_type = other DNA
cccctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggcagcggcgccaccaacttcagcctgctgaaacaggccggcgacgtggaagagaaccctggccctcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattgaggaacccctagtgatggagttggccactccctctctgcgcgctcgctcgctcactgaggccgcccgggcaaagcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacactctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccccagtacggctacctaacactcaacaacggtagtcaggccgtgggacgctcctccttttactgcctggaatatttcccatcgcagatgctgagaacgggcaataactttgagttcagctacagcttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttaccttacccgt

SEQ ID NO: 44 - protein, 8 aa, synthetic construct
  REGION 1..8: note = Synthetic: consensus motif
DVEXNPGP

SEQ ID NO: 45 - DNA, 19 bp, synthetic construct
  misc_feature 1..19: note = Synthetic: F1m
  source 1..19: mol_type = other DNA
atgttccacgaagaagcca

SEQ ID NO: 46 - DNA, 19 bp, synthetic construct
  misc_feature 1..19: note = Synthetic: R1m
  source 1..19: mol_type = other DNA
tcagcaggctgaaattggt

SEQ ID NO: 47 - DNA, 23 bp, synthetic construct
  misc_feature 1..23: note = Synthetic: R2m
  source 1..23: mol_type = other DNA
agctgtttcttactccattctca

SEQ ID NO: 48 - DNA, 24 bp, synthetic construct
  misc_feature 1..24: note = Synthetic: P1m
  source 1..24: mol_type = other DNA
aggcaacgtcatgggtgtgacttt

SEQ ID NO: 49 - DNA, 21 bp, synthetic construct
  misc_feature 1..21: note = Synthetic: F1h
  source 1..21: mol_type = other DNA
gctctcctgcctgttctttag

SEQ ID NO: 50 - DNA, 19 bp, synthetic construct
  misc_feature 1..19: note = Synthetic: R1h
  source 1..19: mol_type = other DNA
tcagcaggctgaaattggt

SEQ ID NO: 51 - DNA, 22 bp, synthetic construct
  misc_feature 1..22: note = Synthetic: R2h
  source 1..22: mol_type = other DNA
tcagcataataagggcaacact

SEQ ID NO: 52 - DNA, 29 bp, synthetic construct
  misc_feature 1..29: note = Synthetic: P1h
  source 1..29: mol_type = other DNA
gcaagaactgtcaattcaagctagcaact

SEQ ID NO: 53 - DNA, 23 bp, synthetic construct
  misc_feature 1..23: note = Synthetic: F2m
  source 1..23: mol_type = other DNA
cacacttccagagaaggagaagc

SEQ ID NO: 54 - DNA, 19 bp, synthetic construct
  misc_feature 1..19: note = Synthetic: R3m
  source 1..19: mol_type = other DNA
tcagcaggctgaagttggt

SEQ ID NO: 55 - DNA, 23 bp, synthetic construct
  misc_feature 1..23: note = Synthetic: P2m
  source 1..23: mol_type = other DNA
aagacgccttagccggcagcggc

SEQ ID NO: 56 - DNA, 24 bp, synthetic construct
  misc_feature 1..24: note = Synthetic: F2h
  source 1..24: mol_type = other DNA
tgagaaggagagacaaatcaagaa

SEQ ID NO: 57 - DNA, 17 bp, synthetic construct
  misc_feature 1..17: note = Synthetic: R3h
  source 1..17: mol_type = other DNA
tcgccggcctgtttcag

SEQ ID NO: 58 - DNA, 20 bp, synthetic construct
  misc_feature 1..20: note = Synthetic: P2h
  source 1..20: mol_type = other DNA
ttaggcttaggaagcggcgc